SEQUENCE LISTING (222 sequences)

SEQ ID NO: 1 - DNA, 1020 bp, Homo sapiens
ggccttccaaagtgctgggattacaggcgtgagtcaccgcgcccggccaaataaaataaaatgttaaagcaaattcaggactacccctcctccaagtcttctgttccctttgggcgcccaggtgagcgggggaggggctgggggagtaataacatcaaaagagcgccttttcctcccttattccgaggagacttccctgggcctgactcccggtcctgtccccagcgccccgcggcctctggagccccttcagtgaccaagatacagagatcaggacgcctttgcgccgccccaggtgcccgcccctagctggctctgcttgggccgcgagggaaggtgaggtcgggggcggagccggggcgtgacagccggggtgtgtgtccgccgggcttggtgcctccggtggccctgcagcaccgtcccacctctgccaccctccgatggggccgctacctgtgtgcctgccaatcatgctgctcctgctactgccgtcgctgctgctgctgctgcttctacctggccccgggtccggcgaggcctccaggatattacgtgtgcaccggcgtgggatcctggaactggcaggaactgtgggttgtgttggtccccgaacccccatcgcctatatgaaatatggttgcttttgtggcttgggaggccatggccagccccgcgatgccattgactggtgctgccatggccacgactgttgttacactcgagctgaggaggccggctgcagccccaagacagagcgctactcctggcagtgcgtcaatcagagcgtcctgtgcggaccggcagagaacaaatgccaagaactgttgtgcaagtgtgaccaggagattgctaactgcttagcccaaactgagtacaacttaaagtacctcttctacccccagttcctatgtgagccggactcgcccaagtgtgactgactaccttgacttgaaatgctcttttgcacaaggaaataaagcgtcctctcagtaatgaaaaaaaaaaaaaaaaaaaaaaaaa

SEQ ID NO: 2 - protein, 165 aa, Homo sapiens
MGPLPVCLPIMLLLLLPSLLLLLLLPGPGSGEASRILRVHRRGILELAGTVGCVGPRTPIAYMKYGCFCGLGGHGQPRDAIDWCCHGHDCCYTRAEEAGCSPKTERYSWQCVNQSVLCGPAENKCQELLCKCDQEIANCLAQTEYNLKYLFYPQFLCEPDSPKCD

SEQ ID NO: 3 - protein, 118 aa, Homo sapiens
EVQLVQSGAEVKKPGESLKISCKGSGYSFISYWIGWVRQMPGKGLEWMGIIYPGDSDTRYSPSFQGQVTISADKSISTAYLQWSSLKASDTAMYYCARHWSYGMDVWGQGTTVTVSSA

SEQ ID NO: 4 - protein, 109 aa, Homo sapiens
EIVLTQSPGTLSLSPGERATLSCRASQSVRSGYLAWYQQRPGQAPRFLIYGASSRATGIPDRFSGSGSGTDFTLTISRLEPEDFAVYYCQQYGSSPLTFGGGTKVEIKR

SEQ ID NO: 5 - protein, 118 aa, Homo sapiens
EVQLVQSGAEVKKPGESLKISCKGSGYSFISYWIGWVRQMPGKGLEWMGIIYPGDSDTRYSPSFQGQVTISADKSISTAYLQWSSLKASDTAMYYCARSWTYALDVWGQGTAVTVSSA

SEQ ID NO: 6 - protein, 109 aa, Homo sapiens
EIVLTQSPGTLSLSPGERATLSCRPSQSVRSNYLTWYQQKPGQAPRLLIYGASTRATGIPDRFSGSGSGTDFTLTVSRLEPEDFAVYYCQQYGSSPLTFGGGTKVEIKR

SEQ ID NO: 7 - protein, 118 aa, Homo sapiens
EVQLVQSGAEVKKPGESLKISCKGSGYSITSYWIGWVRQMPGKGLEWMGIIYPGDSDTRYSPSFQGQVTISADKSISTAYLQWSSLKASDTAMYYCARHSGSSFDYWGQGTLVTVSSA

SEQ ID NO: 8 - protein, 112 aa, Homo sapiens
DIVMTQSPLSLPVTPGEPASISCRSSQSLLHSNGYNYLDWYLQKPGQSPQLLIYLGSYRASGVPDRFSGSGSGTDFTLKISRVEAEDAGVYFCMQGLKTITFGQGTRLEIKR

SEQ ID NO: 9 - protein, 117 aa, Homo sapiens
EVQLVQSGAEVKKPGESLKISCKGSGYSFTNYWINWVRQMPGKGLEWMGIIYPGDSDTRYSPSFQGQVTISADKSISTAYLQWSSLKASDTAMYYCARHRLGFDYWGQGTLVTVSSA

SEQ ID NO: 10 - protein, 108 aa, Homo sapiens
DIQMTQSPSSLSASVGDRVTITCRASQGIRNDLDWCQQKPGKAPKRLIYAASSLQSGVPSRFSGSGSGTEFTLTISSLQPEDFATYYCLQHNNYPPTFGQGTKVEIKR

SEQ ID NO: 11 - protein, 117 aa, Homo sapiens
EVQLVQSGAEVKKPGESLKISCKGSGYRFTSYWISWVRQMPGKGLEWMGIIYPGDSDTRYSPSFQGQVTISADKSISTAYLQWSSLKASDTAMYYCARHREAFDIWGQGTMVTVSSA

SEQ ID NO: 12 - protein, 113 aa, Homo sapiens
DIVMTQSPLSLPVTPGEPASISCRSSQSLLHSNGYNFLDWYLQKPGQSPQLLIYLGSNRASGVPDRFSGSGSGTDFTLKISRVEAEDVGVYYCMQALQTPPTFGPGTKVEIKR

SEQ ID NO: 13 - protein, 118 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSWTYGMDVWGQGTTVTVSSA

SEQ ID NO: 14 - protein, 109 aa, Homo sapiens
EIVLTQSPGTLSLSPGERATLSCRASQSVRSNYLTWYQQKPGQAPRLLIYGASTRATGIPDRFSGSGSGTDFTLTISRLEPEDFAVYYCQQYGSSPLTFGGGTKVEIKR

SEQ ID NO: 15 - protein, 118 aa, Homo sapiens
GVQLVQSGAEVKKPGESLKISCKGSGYSFTNYWIGWVRQMPGKGLEWMGIIYPGDSDTRYSPSFQGQVTISADKSISTAYLQWSSLKASDTAIYYCARGGVGAFDIWGQGTMVTVSSA

SEQ ID NO: 16 - protein, 110 aa, Homo sapiens
EIVLTQSPGTLSLSPGERATLSCRASQIIRRSSLAWYQEKPGQAPRLLIYGASSRATGIPDRFSGSGSGTDFTLTISRLEPEDFAVYYCQQYGSSPPFTFGPGTKVDIKR

SEQ ID NO: 17 - protein, 118 aa, Homo sapiens
EVQLVQSGAGVKKPGESLKISCKGSGYSFTSYWINWVRQMPGKGLEWMGIIYPGDSDTRYSPSFQGQVTISADKSISTAYLQWSSLKASDTAMYYCARSTSSAFDIWGQGTMVTVSSA

SEQ ID NO: 18 - protein, 108 aa, Homo sapiens
DIQMTQSPSSLSASVGDRVTITCRASQSISRYLNWYQQKPGKAPKLLIYAASSLQSGVPSRFSGSGSGTDFTLTISSLQPEDFATYYCQQSYSTPPTFGQGTKVEIKR

SEQ ID NO: 19 - protein, 118 aa, Homo sapiens
EVQLVQSGAEVKKPGESLKISCKGSGYNFITYWIAWVRQMPGKGLEWMGIIYPGDSDTRYSPSFQGQVTISADKSISTAYLQWSSLKASDTAMYYCALTGTRAFEIWGQGTMVTVSSA

SEQ ID NO: 20 - protein, 111 aa, Homo sapiens
DIQMTQSPSSLSASVGDRVTITCRASQSIGSYLNWYQQKPGKPGKGPKLLIYAASTLQSGVPSRFSGGGSGTDFTLTIRSLQPEDFATYYCQQSFNTPPTFGPGTKVDIKR

SEQ ID NO: 21 - protein, 119 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARRDWNYAFDIWGQGTMVTVSSA

SEQ ID NO: 22 - protein, 108 aa, Homo sapiens
DIQMTQSPSSLSASVGDRVTITCRASQSISNYLNWYQQKPGKAPKFLIYAASSLQSGAPSRFSGSGSGTDFTLTISSLQPEDFATYYCQQSYSTPITFGQGTRLEIKR

SEQ ID NO: 23 - protein, 121 aa, Homo sapiens
QVQLVESGGGVVQPGRSLRLSCAASGFTFSSYGMHWVRQAPGKGLEWVAAIWYDGSNKWYADSVKGRFTISRDNSKNTLYLQMNSLRAEDTAVYYCARGGTGTPGAFDIWGQGTMVTVSSA

SEQ ID NO: 24 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRMEAEDVGVYYCMQALQTITFGQGTRLEIKR

SEQ ID NO: 25 - protein, 121 aa, Homo sapiens
QVQLEESGGGVVQPGRSLRLSCAASGFTFSSYGMHWVRQGPGKGLEWVAVIWYDGSNKKYADSVKGRFTISRDNSKNTLYLQMNSLRAEDTAVYYCARDGPIFGVVMGYWGQGTLVTVSSA

SEQ ID NO: 26 - protein, 108 aa, Homo sapiens
DIQMTQSPSSLSASVGDRVTITCRTSQSISNYLNWFQQKPGKAPILLIYAASSLQSGVPSRFSGSGSGTDFTLTISSLQPEDFATYYCHQSYSIPITFGQGTRLEIKR

SEQ ID NO: 27 - protein, 118 aa, Homo sapiens
EVQLVQSGAEVKKPGESLKISCKGSGYSFISYWIAWVRQMPGKGLEWMGIIYPGDSDARYSPSFQGQVTISADKSISTAYLQWSSLKASDTAMYYCARTTSDAFDIWGQGTMVTVSSA

SEQ ID NO: 28 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYNTPPTFGQGTKVEIKR

SEQ ID NO: 29 - protein, 118 aa, Homo sapiens
EVQLVQSGAEVKKPGESLKISCKGSGYSFTIYWIGWVRQMPGKGLEWMGIIYPGDSDTRYSPSFQGQVTISADQSISTAYLQWSSLKASDTAMYYCARHDSYGMDVWGQGTTVTVSSA

SEQ ID NO: 30 - protein, 118 aa, Homo sapiens
  VARIANT 102: Xaa = Any Amino Acid
EVQLVQSGAEVKKPGESLKISCKGSGYSFTSYWIGWLRQMPGKGLEWMGIIYPGDSDTRYSPSFQGQVTISADKSISTAYLQWSSLKASDTAMYYCARSTSXAFDIWGQGTMVTVSSA

SEQ ID NO: 31 - protein, 118 aa, Homo sapiens
EVQLVQSGAEVKKPGESLKISCKGSGYSFTSYWINWVRQMPGKGLEWMGIIYPGDSDTRYSPSFQGQVTISADKSISTAYLQWSSLKASDTAMYYCARHVRSPFDYWGQGTLVTVSSA

SEQ ID NO: 32 - DNA, 23 bp, Homo sapiens
  misc_feature 21: n = inosine
caggtgcagctggagcagtcngg

SEQ ID NO: 33 - DNA, 24 bp, Homo sapiens
gctgagggagtagagtcctgagga

SEQ ID NO: 34 - DNA, 19 bp, Homo sapiens
cacaccgcggtcacatggc

SEQ ID NO: 35 - DNA, 20 bp, Homo sapiens
ctactctagggcacctgtcc

SEQ ID NO: 36 - DNA, 11 bp, Homo sapiens
tgggacctact

SEQ ID NO: 37 - DNA, 15 bp, Homo sapiens
ggatacagctatggt

SEQ ID NO: 38 - DNA, 16 bp, Homo sapiens
gtatagcggtggctgg

SEQ ID NO: 39 - DNA, 15 bp, Homo sapiens
tatagtagctcgtcc

SEQ ID NO: 40 - DNA, 15 bp, Homo sapiens
atagcagcagctggt

SEQ ID NO: 41 - DNA, 12 bp, Homo sapiens
gggtatagcagt

SEQ ID NO: 42 - DNA, 10 bp, Homo sapiens
tccttttaaa

SEQ ID NO: 43 - DNA, 10 bp, Homo sapiens
ctggaactac

SEQ ID NO: 44 - DNA, 15 bp, Homo sapiens
ggatacagctatggt

SEQ ID NO: 45 - DNA, 13 bp, Homo sapiens
cagtggctggtac

SEQ ID NO: 46 - DNA, 10 bp, Homo sapiens
ctggaactac

SEQ ID NO: 47 - DNA, 20 bp, Homo sapiens
tatgattacgtttgggggag

SEQ ID NO: 48 - DNA, 15 bp, Homo sapiens
ggatacagctatggt

SEQ ID NO: 49 - DNA, 10 bp, Homo sapiens
agggactgga

SEQ ID NO: 50 - DNA, 15 bp, Homo sapiens
ggatacagctatggt

SEQ ID NO: 51 - protein, 10 aa, Homo sapiens
GFTFSSYAMN

SEQ ID NO: 52 - protein, 17 aa, Homo sapiens
FISGSGGSTYYADSVKG

SEQ ID NO: 53 - protein, 9 aa, Homo sapiens
KGDWNYEDY

SEQ ID NO: 54 - protein, 10 aa, Homo sapiens
GYSFTSYWIG

SEQ ID NO: 55 - protein, 17 aa, Homo sapiens
IIYPGDSDTRYSPSFQG

SEQ ID NO: 56 - protein, 8 aa, Homo sapiens
LGPTPFDY

SEQ ID NO: 57 - protein, 10 aa, Homo sapiens
GYTFTDYYIH

SEQ ID NO: 58 - protein, 17 aa, Homo sapiens
WIHPNSGGTNYAQKFQG

SEQ ID NO: 59 - protein, 17 aa, Homo sapiens
DRDTAMVFYYYYYAMDV

SEQ ID NO: 60 - protein, 12 aa, Homo sapiens
GDSVSSNSAAWN

SEQ ID NO: 61 - protein, 18 aa, Homo sapiens
RTYYRSKWYNDYAVSVKS

SEQ ID NO: 62 - protein, 16 aa, Homo sapiens
GEYSGGWNFYYYGMDV

SEQ ID NO: 63 - protein, 10 aa, Homo sapiens
GFTFSSYAMS

SEQ ID NO: 64 - protein, 17 aa, Homo sapiens
AISGSGGSTYYADSVKG

SEQ ID NO: 65 - protein, 13 aa, Homo sapiens
EGVTTIFYWYFDL

SEQ ID NO: 66 - protein, 12 aa, Homo sapiens
GGSISSGGYYWS

SEQ ID NO: 67 - protein, 16 aa, Homo sapiens
YIYYSGSTYYNPSLKS

SEQ ID NO: 68 - protein, 11 aa, Homo sapiens
EVIVARPWFDP

SEQ ID NO: 69 - protein, 10 aa, Homo sapiens
GFTFSIYGMH

SEQ ID NO: 70 - protein, 17 aa, Homo sapiens
IISYGGSNKYYADSVKG

SEQ ID NO: 71 - protein, 12 aa, Homo sapiens
EIAAAGSSGMDV

SEQ ID NO: 72 - protein, 10 aa, Homo sapiens
GYSFTSYWIG

SEQ ID NO: 73 - protein, 17 aa, Homo sapiens
IIYPGDSDTRYSPSFQG

SEQ ID NO: 74 - protein, 12 aa, Homo sapiens
PPPGIAVPFKDY

SEQ ID NO: 75 - protein, 10 aa, Homo sapiens
GFTFSSYGMH

SEQ ID NO: 76 - protein, 17 aa, Homo sapiens
IIWYDGSYRFYADSVKG

SEQ ID NO: 77 - protein, 5 aa, Homo sapiens
RGFDY

SEQ ID NO: 78 - protein, 10 aa, Homo sapiens
GFTFSSYSMN

SEQ ID NO: 79 - protein, 17 aa, Homo sapiens
YISSGSSTIYYADSVKG

SEQ ID NO: 80 - protein, 17 aa, Homo sapiens
EGLELRRGYYYYYGMDV

SEQ ID NO: 81 - protein, 10 aa, Homo sapiens
GYTFTGYYMH

SEQ ID NO: 82 - protein, 17 aa, Homo sapiens
WINPNSGGTNYAQKFQG

SEQ ID NO: 83 - protein, 17 aa, Homo sapiens
DRDTAMVFYYYYYALDV

SEQ ID NO: 84 - protein, 10 aa, Homo sapiens
GFTFSSYAMS

SEQ ID NO: 85 - protein, 17 aa, Homo sapiens
AISGSGGSTYYADSVKG

SEQ ID NO: 86 - protein, 13 aa, Homo sapiens
EGVTTIFYWYFDL

SEQ ID NO: 87 - protein, 10 aa, Homo sapiens
GYSFTSYWIG

SEQ ID NO: 88 - protein, 17 aa, Homo sapiens
IIYPGDSDTRYSPSFQG

SEQ ID NO: 89 - protein, 7 aa, Homo sapiens
QRRGFDY

SEQ ID NO: 90 - protein, 10 aa, Homo sapiens
GYSFTSYWIA

SEQ ID NO: 91 - protein, 17 aa, Homo sapiens
IIYPGDSDTRYSPSFQG

SEQ ID NO: 92 - protein, 7 aa, Homo sapiens
GRGGFDY

SEQ ID NO: 93 - protein, 10 aa, Homo sapiens
GFTFSTYGMH

SEQ ID NO: 94 - protein, 17 aa, Homo sapiens
VIWYDGSNKYYADSVKG

SEQ ID NO: 95 - protein, 10 aa, Homo sapiens
AVAGTGAFDI

SEQ ID NO: 96 - protein, 10 aa, Homo sapiens
GFTFSSYSMN

SEQ ID NO: 97 - protein, 17 aa, Homo sapiens
YISSGSSTIYYADSVKG

SEQ ID NO: 98 - protein, 17 aa, Homo sapiens
EGLELRRGYYYYYGMDV

SEQ ID NO: 99 - protein, 12 aa, Homo sapiens
GGSISRSSYYWG

SEQ ID NO: 100 - protein, 16 aa, Homo sapiens
SIYYSGSTYYNPSLKS

SEQ ID NO: 101 - protein, 10 aa, Homo sapiens
GFTFSNYGIH

SEQ ID NO: 102 - protein, 17 aa, Homo sapiens
VIWYDGSYKFYADSVKG

SEQ ID NO: 103 - protein, 5 aa, Homo sapiens
RGFDS

SEQ ID NO: 104 - protein, 10 aa, Homo sapiens
GFTFSSYGMH

SEQ ID NO: 105 - protein, 17 aa, Homo sapiens
AIWYDGSNKYYADSVKG

SEQ ID NO: 106 - protein, 11 aa, Homo sapiens
GGTGTPGAFDI

SEQ ID NO: 107 - protein, 10 aa, Homo sapiens
GFIFSNAWMS

SEQ ID NO: 108 - protein, 19 aa, Homo sapiens
RIKSKTDGGTTDYAAPVKG

SEQ ID NO: 109 - protein, 12 aa, Homo sapiens
GMITFGGAMFDF

SEQ ID NO: 110 - protein, 10 aa, Homo sapiens
GYTFNDYYMH

SEQ ID NO: 111 - protein, 17 aa, Homo sapiens
WIHPNSGGTNYAQKFQG

SEQ ID NO: 112 - protein, 17 aa, Homo sapiens
DRDTAMVFYYYYYAMDV

SEQ ID NO: 113 - protein, 10 aa, Homo sapiens
GFTFRSYGMH

SEQ ID NO: 114 - protein, 17 aa, Homo sapiens
VISYDGSNKYYADSVKG

SEQ ID NO: 115 - protein, 8 aa, Homo sapiens
GVYGDFDY

SEQ ID NO: 116 - protein, 10 aa, Homo sapiens
GFTFSNYGMH

SEQ ID NO: 117 - protein, 17 aa, Homo sapiens
VIWYDGSNKYYADSVKG

SEQ ID NO: 118 - protein, 9 aa, Homo sapiens
RDWNYGMDV

SEQ ID NO: 119 - protein, 10 aa, Homo sapiens
GYTFTDYYMH

SEQ ID NO: 120 - protein, 17 aa, Homo sapiens
WISPNSGGTNYAQKFQG

SEQ ID NO: 121 - protein, 17 aa, Homo sapiens
DRDTAMVFYYYYYAMDV

SEQ ID NO: 122 - protein, 10 aa, Homo sapiens
GFTFSSYGMH

SEQ ID NO: 123 - protein, 17 aa, Homo sapiens
VIWYDGSNKYYADSVKG

SEQ ID NO: 124 - protein, 16 aa, Homo sapiens
QGIAARRNYYYSGMDV

SEQ ID NO: 125 - protein, 10 aa, Homo sapiens
GYTFTSYDIN

SEQ ID NO: 126 - protein, 17 aa, Homo sapiens
WMDPNSGHTGYAQKFQG

SEQ ID NO: 127 - protein, 9 aa, Homo sapiens
EGNWGSFDY

SEQ ID NO: 128 - protein, 10 aa, Homo sapiens
GYSFTNYWIG

SEQ ID NO: 129 - protein, 17 aa, Homo sapiens
FIYPGDSDTRYSPSFEG

SEQ ID NO: 130 - protein, 7 aa, Homo sapiens
HTGALDY

SEQ ID NO: 131 - protein, 10 aa, Homo sapiens
GITFSSYGMH

SEQ ID NO: 132 - protein, 17 aa, Homo sapiens
VIWYDGSNKYYVDSVKG

SEQ ID NO: 133 - protein, 9 aa, Homo sapiens
RGPLYAFDI

SEQ ID NO: 134 - protein, 118 aa, Homo sapiens
  VARIANT 101, 102: Xaa = Any Amino Acid
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGGXXAFDIWGQGTMVTVSSA

SEQ ID NO: 135 - protein, 118 aa, Homo sapiens
  VARIANT 102: Xaa = Any Amino Acid
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSSSXAFDIWGQGTMVTVSSA

SEQ ID NO: 136 - protein, 121 aa, Homo sapiens
  VARIANT 99, 100, 103, 104, 105: Xaa = Any Amino Acid
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARXXTGXXXAFDIWGQGTMVTVSSA

SEQ ID NO: 137 - protein, 119 aa, Homo sapiens
  VARIANT 99, 100: Xaa = Any Amino Acid
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARXXWNYAFDIWGQGTMVTVSSA

SEQ ID NO: 138 - protein, 117 aa, Homo sapiens
  VARIANT 99, 100, 102: Xaa = Any Amino Acid
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARXXLXFDYWGQGTLVTVSSA

SEQ ID NO: 139 - protein, 118 aa, Homo sapiens
  VARIANT 101: Xaa = Any Amino Acid
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARSWXYGMDVWGQGTTVTVSSA

SEQ ID NO: 140 - protein, 118 aa, Homo sapiens
  VARIANT 99: Xaa = Any Amino Acid
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARXWCYGMDVWGQGTTVTVSSA

SEQ ID NO: 141 - protein, 118 aa, Homo sapiens
  VARIANT 97, 98, 102: Xaa = Any Amino Acid
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCXXTGTXAFDIWGQGTMVTVSSA

SEQ ID NO: 142 - protein, 121 aa, Homo sapiens
  VARIANT 99, 100: Xaa = Any Amino Acid
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARXXTIFGVVIDYWGQGTLVTVSSA

SEQ ID NO: 143 - protein, 118 aa, Homo sapiens
  VARIANT 99, 100: Xaa = Any Amino Acid
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARXXYYGMDVWGQGTTVTVSSA

SEQ ID NO: 144 - protein, 117 aa, Homo sapiens
  VARIANT 99, 100, 101: Xaa = Any Amino Acid
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARXXXAFDIWGQGTMVTVSSA

SEQ ID NO: 145 - protein, 118 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARHSGSYFDYWGQGTLVTVSSA

SEQ ID NO: 146 - protein, 12 aa, Homo sapiens
RASQSVSSRYLA

SEQ ID NO: 147 - protein, 7 aa, Homo sapiens
GASSRAT

SEQ ID NO: 148 - protein, 9 aa, Homo sapiens
QQYGSSQIT

SEQ ID NO: 149 - protein, 11 aa, Homo sapiens
RASQGISNDLA

SEQ ID NO: 150 - protein, 7 aa, Homo sapiens
AASSLQS

SEQ ID NO: 151 - protein, 9 aa, Homo sapiens
LQHNSYPLT

SEQ ID NO: 152 - protein, 11 aa, Homo sapiens
RASQGIRNDLG

SEQ ID NO: 153 - protein, 7 aa, Homo sapiens
AASSLQS

SEQ ID NO: 154 - protein, 9 aa, Homo sapiens
LQHNIYPLT

SEQ ID NO: 155 - protein, 17 aa, Homo sapiens
KSSQSVLYSSNNKNYLT

SEQ ID NO: 156 - protein, 7 aa, Homo sapiens
WASTRES

SEQ ID NO: 157 - protein, 9 aa, Homo sapiens
QQYYSTPRT

SEQ ID NO: 158 - protein, 12 aa, Homo sapiens
RASQSVSSRYLA

SEQ ID NO: 159 - protein, 7 aa, Homo sapiens
GASSRAA

SEQ ID NO: 160 - protein, 10 aa, Homo sapiens
QQCDYSPPCS

SEQ ID NO: 161 - protein, 12 aa, Homo sapiens
RASQSVRKSYLA

SEQ ID NO: 162 - protein, 7 aa, Homo sapiens
GASSRAT

SEQ ID NO: 163 - protein, 9 aa, Homo sapiens
QQYDYSPIT

SEQ ID NO: 164 - protein, 17 aa, Homo sapiens
KSSQSVLYSSNNKNYLA

SEQ ID NO: 165 - protein, 7 aa, Homo sapiens
WASTRES

SEQ ID NO: 166 - protein, 9 aa, Homo sapiens
QQYYSTPRT

SEQ ID NO: 167 - protein, 16 aa, Homo sapiens
RSSQSLLQSNGYKYLE

SEQ ID NO: 168 - protein, 7 aa, Homo sapiens
LGSNRAS

SEQ ID NO: 169 - protein, 9 aa, Homo sapiens
MQALQTPLT

SEQ ID NO: 170 - protein, 11 aa, Homo sapiens
RASQSVSSNLA

SEQ ID NO: 171 - protein, 7 aa, Homo sapiens
GASTRAT

SEQ ID NO: 172 - protein, 10 aa, Homo sapiens
QQYNNWPPCS

SEQ ID NO: 173 - protein, 11 aa, Homo sapiens
RASQSVSRILA

SEQ ID NO: 174 - protein, 7 aa, Homo sapiens
GASTRAT

SEQ ID NO: 175 - protein, 9 aa, Homo sapiens
QQYHNWPIT

SEQ ID NO: 176 - protein, 16 aa, Homo sapiens
RSSQSLLHSNGYNYLD

SEQ ID NO: 177 - protein, 7 aa, Homo sapiens
LGSNRAS

SEQ ID NO: 178 - protein, 9 aa, Homo sapiens
MQALQTPFT

SEQ ID NO: 179 - protein, 11 aa, Homo sapiens
QASQDISNYLN

SEQ ID NO: 180 - protein, 7 aa, Homo sapiens
DASNLET

SEQ ID NO: 181 - protein, 9 aa, Homo sapiens
QQYDNLPIT

SEQ ID NO: 182 - protein, 17 aa, Homo sapiens
KSSQSVLYSSNNKYFLA

SEQ ID NO: 183 - protein, 7 aa, Homo sapiens
WASTRES

SEQ ID NO: 184 - protein, 9 aa, Homo sapiens
QQYYSSPWT

SEQ ID NO: 185 - protein, 17 aa, Homo sapiens
KSSQSVLYRSNNKNFLA

SEQ ID NO: 186 - protein, 7 aa, Homo sapiens
WASTRES

SEQ ID NO: 187 - protein, 9 aa, Homo sapiens
QQHYSIPLT

SEQ ID NO: 188 - protein, 17 aa, Homo sapiens
KSSQSVLYSSNNKNYLA

SEQ ID NO: 189 - protein, 7 aa, Homo sapiens
WASTRDS

SEQ ID NO: 190 - protein, 9 aa, Homo sapiens
QQYYSTPRT

SEQ ID NO: 191 - protein, 11 aa, Homo sapiens
RASQGIRNDLA

SEQ ID NO: 192 - protein, 7 aa, Homo sapiens
AASSLQS

SEQ ID NO: 193 - protein, 9 aa, Homo sapiens
LQHNSYPPT

SEQ ID NO: 194 - protein, 12 aa, Homo sapiens
RASQSVSSSYLA

SEQ ID NO: 195 - protein, 7 aa, Homo sapiens
GASSRAT

SEQ ID NO: 196 - protein, 10 aa, Homo sapiens
QHYGSLPPCS

SEQ ID NO: 197 - protein, 16 aa, Homo sapiens
KSSQSLLYSDGKTYLY

SEQ ID NO: 198 - protein, 7 aa, Homo sapiens
EVSNRFS

SEQ ID NO: 199 - protein, 9 aa, Homo sapiens
MQSIQLPLT

SEQ ID NO: 200 - protein, 17 aa, Homo sapiens
KSSQSVLFRSNNRNYLA

SEQ ID NO: 201 - protein, 7 aa, Homo sapiens
WASTRES

SEQ ID NO: 202 - protein, 9 aa, Homo sapiens
QQYYSIPRT

SEQ ID NO: 203 - protein, 16 aa, Homo sapiens
KSSQSLLHSDGKTYLY

SEQ ID NO: 204 - protein, 7 aa, Homo sapiens
EVSNRFS

SEQ ID NO: 205 - protein, 9 aa, Homo sapiens
MQSIQLPLT

SEQ ID NO: 206 - protein, 16 aa, Homo sapiens
RSSQSLLHSNGYNYLD

SEQ ID NO: 207 - protein, 7 aa, Homo sapiens
LGSNRAS

SEQ ID NO: 208 - protein, 8 aa, Homo sapiens
MQALQTIT

SEQ ID NO: 209 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGQGTRLEIKR

SEQ ID NO: 210 - protein, 109 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKR

SEQ ID NO: 211 - protein, 108 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPPTFGQGTKVEIKR

SEQ ID NO: 212 - protein, 113 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPPTFGQGTKVEIKR

SEQ ID NO: 213 - protein, 110 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPFTFGPGTKVDIKR

SEQ ID NO: 214 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKR

SEQ ID NO: 215 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTITFGQGTRLEIKR

SEQ ID NO: 216 - protein, 111 aa, Homo sapiens
  VARIANT 43, 44, 45, 46: Xaa = Any Amino Acid
DIQMTQSPSSLSASVGDRVTITCRASQSISSYLNWYQQKPGKXXXXPKLLIYAASSLQSGVPSRFSGSGSGTDFTLTISSLQPEDFATYYCQQSYSTPPTFGPGTKVDIKR

SEQ ID NO: 217 - protein, 110 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARWGQGTMVTVSSA

SEQ ID NO: 218 - protein, 110 aa, Homo sapiens
EVQLVQSGAEVKKPGESLKISCKGSGYSFISYWIAWVRQMPGKGLEWMGIIYPGDSDARYSPSFQGQVTISADKSISTAYLQWSSLKASDTAMYYCARTTQDTMVTVSSA

SEQ ID NO: 219 - protein, 110 aa, Homo sapiens
EVQLVQSGAEVKKPGESLKISCKGSGYNFITYWIAWVRQMPGKGLEWMGIIYPGDSDTRYSPSFQGQVTISADKSISTAYLQWSSLKASDTAMYYCALWGQRTMETVSSA

SEQ ID NO: 220 - protein, 111 aa, Homo sapiens
  VARIANT 44, 45, 46: Xaa = Any Amino Acid
DIQMTQSPSSLSASVGDRVTITCRASQSISSYLNWYQQKPGKAXXXPKLLIYAASSLQSGVPSRFSGSGSGTDFTLTISSLQPEDFATYYCQQSYSTPPTFGQGTKVEIKR

SEQ ID NO: 221 - protein, 111 aa, Homo sapiens
  VARIANT 44, 45, 46: Xaa = Any Amino Acid
DIQMTQSPSSLSASVGDRVTITCRASQSISSYLNWYQQKPGKAXXXPKLLIYAASSLQSGVPSRFSGSGSGTDFTLTISSLQPEDFATYYCQQSYNTPPTFGQGTKVEIKR

SEQ ID NO: 222 - protein, 111 aa, Homo sapiens
DIQMTQSPSSLSASVGDRVTITCRASQSIGSYLNWYQQKPGKPGKGPKLLIYAASSLQTGVPSRFSGSGSGTDFTLTISSLRPEDFATYYCQQSFNTPPTFGPGTKVDIKR

(19) United States
(12) Patent Application Publication
(10) Pub. No.: US 2005/0058649 A1
(43) Pub. Date: Mar. 17, 2005

(54) Antibodies directed to phospholipase A2 and uses thereof

(76) Inventors: Gregory M. Landes, The Woodlands, TX (US); Mary Haak-Frendscho, Newark, CA (US); Ling Chen, The Woodlands, TX (US); Yen-Wah Rozanne Lee, Newark, CA (US); Meina Liang, Danville, CA (US); Xiao Feng, Union City, CA (US); Xiao-Chi Jia, Los Angeles, CA (US); Mark R. Nocerini, San Francisco, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/726,332

(22) Filed: Dec. 2, 2003

Related U.S. Application Data
(60) Provisional application No. 60/430,724, filed on Dec. 2, 2002.

(51) Int. Cl. A61K 39/395; C07K 16/40

(52) U.S. Cl. 424/146.1; 530/388.26

(57) ABSTRACT

The invention described herein relates to antibodies directed to the antigen phospholipase A2 (PLA2) and uses of such antibodies. In particular, in accordance with some embodiments of the invention, there are provided fully human monoclonal antibodies directed to the antigen PLA2. Nucleotide sequences encoding, and amino acid sequences comprising, heavy and light chain immunoglobulin molecules, particularly sequences corresponding to contiguous heavy and light chain sequences spanning the framework regions and/or complementarity determining regions (CDRs), specifically from FR1 through FR4 or CDR1 through CDR3, are provided. Hybridomas or other cell lines expressing such immunoglobulin molecules and monoclonal antibodies are also provided.

### CROSS REFERENCE AND RELATED APPLICATION

This application claims priority under 35 U.S.C. § 119(e) to U.S. Provisional Application No. 60/430,724, filed Dec. 2, 2002, which is hereby expressly incorporated by reference.

### BACKGROUND OF THE INVENTION

1. Field of the Invention

The invention described herein relates to antibodies directed to the antigen phospholipase A2 (PLA2) and uses of such antibodies. In particular, in accordance with some embodiments of the invention, there are provided fully human monoclonal antibodies directed to the antigen PLA2. Nucleotide sequences encoding, and amino acid sequences comprising, heavy and light chain immunoglobulin molecules, particularly sequences corresponding to contiguous heavy and light chain sequences spanning the framework regions and/or complementarity determining regions (CDRs), specifically from FR1 through FR4 or CDR1 through CDR3, are provided. Hybridomas or other cell lines expressing such immunoglobulin molecules and monoclonal antibodies are also provided.

2. Description of the Related Art

Secreted Phospholipase A2 (PLA2) enzymes are a ubiquitous family of small, disulphide-containing, calcium dependent enzymes that catalyze the hydrolysis of the sn-2 ester bond of phospholipids liberating lysophospholipid and free fatty acid products. See E. A. Dennis, TBE Enzymes, Vol 16, Academic Press, New York (1983). The nucleotide and amino acid sequences of PLA2 are set forth in SEQ ID NOS: 1 and 2, respectively. The side chains of two conserved residues, a histidine and aspartic acid, participate in catalytic sites.

Specifically, PLA2 hydrolyzes the 2-acyl group of L-1,2, diacylphosphatides to generate free fatty acids, such as arachidonic acid and lysophospholipids. While lysophospholipids have the ability to damage cells and membranes, the synthesis of arachidonic acid from membrane phospholipids is the rate-limiting step in the biosynthesis of the four major classes of eicosanoids (prostaglandins, prostacyclins, thromboxanes and leukotrienes) involved in pain, fever, and inflammation. Arachidonic acid is metabolized by two enzymatic pathways and subsequently converted to proinflammatory substances including leukotrienes (via lipoxygenase activity), thromboxanes and prostaglandins (both via cyclooxygenase activity). These chemical mediators recruit cells of the immune system and the complement cascade to produce an exaggerated inflammatory response. Furthermore, leukotriene-B4 is known to function in a feedback loop which further increases PLA2 activity (Wijkander, J. et al. (1995) J. Biol. Chem. 270:26543-26549).

Over eighty PLA2 enzymes have been structurally characterized, and show a high degree of sequence homology. J. Chang, et al., Biochem. Pharm. 36:2429-2436, (1987); F. F. Davidson and E. A. Dennis, J. of Molecular Evolution 31:228-238 (1990). The best-characterized varieties of PLA2 enzymes are the secreted forms, which are released into the extracellular environment where they aid in the digestion of biological materials. The secreted forms have a molecular weight of about 12-15 kDa (Davidson and Dennis, supra).

At least four different groups of PLA2s have been characterized in mammalian cells, including Group I (pancreatic), Groups IIA, and IIC (inflammatory), and Group V (expressed in the heart). Group I PLA2 enzymes function in the digestion of dietary lipids and have been proposed to play a role in cell proliferation, smooth muscle contraction, and acute lung injury. Group II PLA2 enzymes are potent mediators of inflammatory processes and are highly expressed in serum and synovial fluids of patients with inflammatory disorders. These enzymes are found in most human cell types assayed and are expressed in diverse pathological processes such as septic shock, intestinal cancers, rheumatoid arthritis, and epidermal hyperplasia. A Group V PLA2 enzyme has been cloned from brain tissue and is strongly expressed in heart tissue. Other PLA2 enzymes have been cloned from various human tissues and cell lines, suggesting a large diversity of PLA2 enzymes. A human PLA2 enzme was recently cloned from fetal lung, and based on its structural properties, appears to be the first member of a new group of mammalian PLA2 enzymes, referred to as Group X. (Chen J. et al. (1994) J. Biol. Chem. 269:2365-2368; Kennedy, B. P., et al. (1995) J. Biol. Chem. 270:22378-22385; Komada, M., et al. (1990) Biochem. Biophys. Res. Commun. 168:1059-1065; and Cupillard, L. et al. (1997) J. Biol. Chem. 272:15745-15752).

### SUMMARY OF THE INVENTION

Embodiments of the invention relate to antibodies against PLA2. Antibodies directed to the antigen PLA2 are useful as lipid lowering agents, for example in the treatment of atherosclerosis and restenosis. Such antibodies are also useful in the diagnosis, prevention, and treatment of inflammatory disorders. Inflammatory and degenerative disorders account for a significant number of debilitating diseases. Inflammatory states, such as atherosclerosis, arthritis, psoriasis, and asthma stem from inflammatory reactions in the joints, skin, and blood vessels. In addition, recent studies indicate that a major component of the pathology of Alzheimer's disease is chronic inflammation, and administration of nonsteroidal anti-inflammatory drugs appears to slow the advance of Alzheimer's disease. Schnabel, Science 260:1719-1720 (1993).

Attenuating or eliminating the inflammatory response would be the key to the treatment of these diseases. Accordingly, the antibodies described herein act as inhibitors of PLA2 in order to prevent release of arachidonic acid from membrane phospholipids, to stop the entire arachidonic acid cascade, and to thereby cease the destruction attributed to the inflammatory process.

Embodiments of the invention also include monoclonal antibodies that bind PLA2 and affect PLA2 function. Accordingly, embodiments of the invention provide human anti-PLA2 antibodies and anti-PLA2 antibody preparations with desirable properties from diagnostic and therapeutic perspectives. In particular, one embodiment of the invention provides anti-PLA2 antibodies having characteristics that provide therapeutic utility, including, for example, but not limited to, strong binding affinity for PLA2, the ability to neutralize PLA2 function in vitro, and the ability to produce prolonged neutralization of PLA2 function in vivo.

One embodiment of the invention is a fully human monoclonal antibody that binds to PLA2 and has a heavy chain amino acid sequence selected from the group consisting of SEQ ID NOS: 3, 5, 7, 9, 11, 13, 15, 17, 19, 21, 23, 25, 27, 29, 30, and 31. In one embodiment, the antibody further comprises a light chain amino acid sequence selected from the group consisting of SEQ ID NOS: 4, 6, 8, 10, 12, 14, 16, 18, 20, 22, 24, 26, and 28.

Another embodiment of the invention is a fully human antibody that binds to PLA2 and has a heavy chain amino acid sequence having a CDR sequence shown in Tables 3 and 4. It is noted that CDR determinations can be readily accomplished by those of ordinary skill in the art. In general, CDRs are presented in the invention described herein as defined by Kabat et al., in Sequences of Proteins of Immunological Interest vols. 1-3 (Fifth Edition, NIH Publication 91-3242, Bethesda Md. 1991).

Yet another embodiment of the invention is a fully human antibody that binds to PLA2 and comprises a light chain amino acid sequence having a CDR sequence shown in Tables 5 and 6.

A further embodiment of the invention is a fully human antibody that binds to PLA2 and comprises a heavy chain amino acid sequence having the CDRs comprising the sequences shown in Tables 3 and 4 and a light chain amino acid sequence having the CDRs comprising the sequences shown in Tables 5 and 6.

A further embodiment of the invention is an antibody that cross-competes for binding to PLA2 with the fully human antibodies of the invention. In another embodiment of the invention, the fully human antibody is anti-PLA2 mAb 2.12 or anti-PLA2 mAb 2.25.

Embodiments of the invention described herein are based upon the generation and identification of isolated antibodies that bind specifically to PLA2. As discussed above, PLA2 is expressed at elevated levels in inflammatory diseases and related conditions. Inhibition of the biological activity of PLA2 can therefore delay the progression of symptoms caused by such diseases and conditions. The disease or condition can be, for example, inflammatory and degenerative disorders that stem from inflammatory reactions in the joints, skin, and blood vessels, including, without limitation, arthritis, psoriasis, asthma, and Alzheimer's disease, but most preferably, atherosclerosis and restenosis.

Accordingly, one embodiment of the invention described herein provides isolated antibodies, or fragments of those antibodies, that bind to PLA2. As known in the art, the antibodies can advantageously be, for example, monoclonal, chimeric and/or human antibodies. Embodiments of the invention described herein also provide cells for producing these antibodies.

It will be appreciated that embodiments of the invention are not limited to any particular anti-PLA2 antibody, or any specific form of an antibody. For example, the anti-PLA2 antibody may be a full length antibody (e.g. having an intact human Fc region) or an antibody fragment (e.g. a Fab, Fab′ or F(ab′)₂). In addition, the antibody may be manufactured from a hybridoma that secretes the antibody, or from a recombinantly produced cell that has been transformed or transfected with a gene or genes encoding the antibody.

In a preferred embodiment, the invention includes the treatment of inflammatory conditions and related diseases in humans, including but not limited to, inflammatory and degenerative disorders that stem from inflammatory reactions in the joints, skin, and blood vessels, including, without limitation, arthritis, psoriasis, asthma, and Alzheimer's disease, but most preferably, atherosclerosis and restenosis.

In one embodiment, the anti-PLA2 antibody forms a pharmaceutical composition comprising an effective amount of the antibody, or a fragment thereof, in association with a pharmaceutically acceptable carrier or diluent. In another embodiment, the anti-PLA2 antibody or fragment thereof is conjugated to a therapeutic agent. The therapeutic agent can be a toxin or a radioisotope. Preferably, such antibodies can be used for the treatment of diseases, such as, inflammatory and degenerative disorders that stem from inflammatory reactions in the joints, skin, and blood vessels, including, without limitation, arthritis, psoriasis, asthma, and Alzheimer's disease, but most preferably, atherosclerosis and restenosis.

In another embodiment, the invention includes a method for treating diseases or conditions associated with the expression of PLA2 in a patient by administering to the patient an effective amount of an anti-PLA2 antibody. The patient is a mammalian patient, preferably a human patient. The disease or condition can be, for example, inflammatory and degenerative disorders that stem from inflammatory reactions in the joints, skin, and blood vessels, including, without limitation, arthritis, psoriasis, asthma, and Alzheimer's disease, but most preferably, atherosclerosis and restenosis. Additional embodiments include methods for the treatment of diseases or conditions associated with the expression of PLA2 in a mammal by identifying a mammal in need of treatment for an inflammatory condition and administering to the mammal a therapeutically effective dose of anti-PLA2 antibodies.

Alternatively, anti-PLA2 antibodies may be administered to prevent a mammal from contracting diseases or conditions associated with the expression of PLA2 including, but not limited to, inflammatory conditions or related diseases. Preferably the anti-PLA2 antibodies are fully human. The disease or condition can be, for example, but not limited to, inflammatory and degenerative disorders that stem from inflammatory reactions in the joints, skin, and blood vessels, including, without limitation, arthritis, psoriasis, asthma, and Alzheimer's disease, and most preferably, atherosclerosis and restenosis.

In another embodiment, the invention is an article of manufacture including a container having a composition containing an anti-PLA2 antibody, and a package insert or label indicating that the composition can be used to treat conditions characterized by the expression of PLA2. Preferably a mammal and, more preferably, a human, receives the anti-PLA2 antibody. In a preferred embodiment, inflammatory conditions and related diseases in humans are treated.

Another embodiment is a method for identifying risk factors of a disease, diagnosing a disease, and staging a disease, which method involves identifying the presence of PLA2 using anti-PLA2 antibodies.

In one embodiment, the invention includes a method for diagnosing a condition associated with the expression of PLA2 in a cell by contacting the cell with an anti-PLA2 antibody, and detecting the presence of PLA2.

In still another embodiment, the invention includes an assay kit for the detection of PLA2 in mammalian tissues or cells to screen for inflammatory conditions and related diseases in humans. The kit includes an antibody that binds to PLA2 and a means for indicating the reaction of the antibody with PLA2, if present. Preferably the antibody is a monoclonal antibody. In one embodiment, the antibody that binds PLA2 is labeled. Preferably, the antibody is labeled with a marker selected from the group consisting of: a fluorochrome, an enzyme, a radionuclide and a radiopaque material. In another embodiment the antibody is an unlabeled first antibody and the means for indicating the reaction is a labeled anti-immunoglobulin antibody.

Yet another embodiment is the use of an anti-PLA2 antibody in the preparation of a medicament for the treatment of inflammatory conditions and related diseases. In one embodiment, the disease is selected from the group comprising inflammatory and degenerative disorders that stem from inflammatory reactions in the joints, skin, and blood vessels, including, without limitation, arthritis, psoriasis, asthma, and Alzheimer's disease, but most preferably, atherosclerosis and restenosis.

### BRIEF DESCRIPTION OF THE DRAWINGS

FIG. 1A is a bar graph showing a dose-response curve of titrating amounts of substrate incubated with 0.5 units of PLA2 enzyme.

FIG. 1B is a bar graph showing that KLH had minimal effects on the assay.

FIG. 2A is a bar graph showing titrating amounts of Fxa-cleaved bacterially-expressed enzyme incubated with 400 nM Bis-BODIPY® substrate.

FIG. 2B is a bar graph showing enzyme activity of bacterially-expressed PLA2 slightly inhibited by 20 μl/well KLH exhaust supernatant from G2 and G4.

FIG. 3 is a line graph showing percent inhibition at the highest dose tested for each antibody.

FIG. 4 is an alignment of peptide consensus sequences of specific binders of mAb2.12.

### DETAILED DESCRIPTION OF THE PREFERRED EMBODIMENT

One embodiment of the invention relates to antibodies directed to the antigen PLA2 and uses of such antibodies. For example, antibodies against PLA2 may be used in methods for effectively preventing, treating, diagnosing, and/or staging inflammatory conditions and related diseases. Such conditions include, for example, inflammatory reactions in the joints, skin, and blood vessels, atherosclerosis, arthritis, psoriasis, asthma, restinosis, and Alzheimer's disease. In one particular embodiment, a therapeutically effective amount of anti-PLA2 antibodies are administered as a treatment for inflammatory conditions and related diseases. In preferred embodiments, the antibodies are fully human monoclonal antibodies directed to the antigen PLA2.

Other embodiments of the invention relate to other compounds that result in a reduction of inflammation in vivo. Thus, compounds that reduce the level of PLA2 would be useful in treatment of inflammatory conditions. PLA2 nucleic acids, polypeptides, antibodies, agonists, antagonists, and other related compounds' uses are disclosed more fully below.

Additionally, the nucleic acids of the invention, and fragments and variants thereof, may be used, by way of nonlimiting example, (a) to direct the biosynthesis of the corresponding encoded proteins, polypeptides, fragments and variants as recombinant or heterologous gene products, (b) as probes for detection and quantification of the nucleic acids disclosed herein, (c) as sequence templates for preparing antisense molecules, and the like. Such uses are described more fully in the following disclosure.

Furthermore, the proteins and polypeptides of the invention, and fragments and variants thereof, may be used, in ways that include (a) serving as an immunogen to stimulate the production of an anti-PLA2 antibody, (b) a capture antigen in an immunogenic assay for such an antibody, (c) as a target for screening for substances that bind to a PLA2 polypeptide of the invention, and (d) a target for a PLA2-specific antibody such that treatment with the antibody inhibits the inflammatory response. These utilities and other utilities for PLA2 nucleic acids, polypeptides, antibodies, agonists, antagonists, and other related compounds' uses are disclosed more fully below. In view of its strong effects in modulating inflammation, an increase of PLA2 polypeptide expression or activity can be used to promote inflammation. Conversely, a decrease in PLA2 polypeptide expression can be used to reduce inflammation.

Sequence Listing

The heavy chain and light chain variable region nucleotide and amino acid sequences of representative human anti-PLA2 antibodies are provided in the sequence listing, the contents of which are summarized in Table 1 below.

TABLE 1

| mAb | | SEQ ID |
|---|---|---|
| ID No.: | Sequence | NO: |
| 1.5 | Amino acid sequence encoding the variable region of the heavy chain | 3 |
| | Amino acid sequence encoding the variable region of the light chain | 4 |
| 1.7 | Amino acid sequence encoding the variable region of the heavy chain | 5 |
| | Amino acid sequence encoding the variable region of the light chain | 6 |
| 1.14 | Amino acid sequence encoding the variable region of the heavy chain | 7 |
| | Amino acid sequence encoding the variable region of the light chain | 8 |
| 1.18 | Amino acid sequence encoding the variable region of the heavy chain | 9 |
| | Amino acid sequence encoding the variable region of the light chain | 10 |
| 1.21 | Amino acid sequence encoding the variable region of the heavy chain | 11 |
| | Amino acid sequence encoding the variable region of the light chain | 12 |
| 1.27 | Amino acid sequence encoding the variable region of the heavy chain | 13 |
| | Amino acid sequence encoding the variable region of the light chain | 14 |
| 2.7 | Amino acid sequence encoding the variable region of the heavy chain | 15 |
| | Amino acid sequence encoding the variable region of the light chain | 16 |
| 2.9 | Amino acid sequence encoding the variable region of the heavy chain | 17 |
| | Amino acid sequence encoding the variable region of the light chain | 18 |
| 2.12 | Amino acid sequence encoding the variable region of the heavy chain | 19 |
| | Amino acid sequence encoding the variable region of the light chain | 20 |
| 2.15 | Amino acid sequence encoding the variable region of the heavy chain | 21 |
| | Amino acid sequence encoding the variable region of the light chain | 22 |
| 2.19 | Amino acid sequence encoding the variable region of the heavy chain | 23 |
| | Amino acid sequence encoding the variable region of the light chain | 24 |
| 2.23 | Amino acid sequence encoding the variable region of the heavy chain | 25 |
| | Amino acid sequence encoding the variable region of the light chain | 26 |
| 2.25 | Amino acid sequence encoding the variable region of the heavy chain | 27 |
| | Amino acid sequence encoding the variable region of the light chain | 28 |
| 1.3 | Amino acid sequence encoding the variable region of the heavy chain | 29 |
| 2.4 | Amino acid sequence encoding the variable region of the heavy chain | 30 |
| 2.16 | Amino acid sequence encoding the variable region of the heavy chain | 31 |

Definitions

Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the instant specification. See, e.g., Sambrook et al., Molecular Cloning: A Laboratory Manual (2d ed., Cold Spring Harbor Laboratory Press, Cold Spring Harbor, N.Y. 1989), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the embodiments provided herein, the following terms, unless otherwise indicated, shall be understood to have the following meanings:

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g. free of murine proteins, (3) is expressed by a cell from a different species, or (4) does not occur in nature.

The term “polypeptide” is used herein as a generic term to refer to native protein, fragments, or analogs of a polypeptide sequence. Hence, native protein, fragments, and analogs are species of the polypeptide genus. Preferred polypeptides in accordance with the invention comprise the human heavy chain immunoglobulin molecules and the human kappa light chain immunoglobulin molecules, as well as antibody molecules formed by combinations comprising the heavy chain immunoglobulin molecules with light chain immunoglobulin molecules, such as the kappa light chain immunoglobulin molecules, and vice versa, as well as fragments and analogs thereof.

The term “naturally occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory or otherwise is naturally occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences which are necessary to effect the expression and processing of coding sequences to which they are ligated. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring, and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes; although oligonucleotides may be double stranded, e.g. for use in the construction of a gene mutant. Oligonucleotides of the invention can be either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phoshoraniladate, phosphoroamidate, and the like. See e.g., LaPlanche et al. Nucl. Acids Res. 14:9081 (1986); Stec et al. J. Am. Chem. Soc. 106:6077 (1984); Stein et al. Nucl. Acids Res. 16:3209 (1988); Zon et al. Anti-Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990), the disclosures of which are hereby incorporated by reference. An oligonucleotide can include a label for detection, if desired.

The term “selectively hybridize” referred to herein means to detectably and specifically bind. Polynucleotides, oligonucleotides and fragments thereof in accordance with the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest will be at least 80%, and more typically with preferably increasing homologies of at least 85%, 90%, 95%, 99%, and 100%. Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See M. O. Dayhoff, in Atlas of Protein Sequence and Structure, Vol. 5, 101-110 and Supplement 2 to Vol. 5, 1-10 (National Biomedical Research Foundation 1972). The two sequences or parts thereof are more preferably homologous if their amino acids are greater than or equal to 50% identical when optimally aligned using the ALIGN program. The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a “GTATA”.

The following terms are used to describe the sequence relationships between two or more polynucleotide or amino acid sequences: “reference sequence,” “comparison window,” “sequence identity,” “percentage of sequence identity,” and “substantial identity”. A “reference sequence” is a defined sequence used as a basis for a sequence comparison; a reference sequence may be a subset of a larger sequence, for example, as a segment of a full-length cDNA or gene sequence given in a sequence listing or may comprise a complete cDNA or gene sequence. Generally, a reference sequence is at least 18 nucleotides or 6 amino acids in length, frequently at least 24 nucleotides or 8 amino acids in length, and often at least 48 nucleotides or 16 amino acids in length. Since two polynucleotides or amino acid sequences may each (1) comprise a sequence (i.e., a portion of the complete polynucleotide or amino acid sequence) that is similar between the two molecules, and (2) may further comprise a sequence that is divergent between the two polynucleotides or amino acid sequences, sequence comparisons between two (or more) molecules are typically performed by comparing sequences of the two molecules over a “comparison window” to identify and compare local regions of sequence similarity. A “comparison window,” as used herein, refers to a conceptual segment of at least 18 contiguous nucleotide positions or 6 amino acids wherein a polynucleotide sequence or amino acid sequence may be compared to a reference sequence of at least 18 contiguous nucleotides or 6 amino acid sequences and wherein the portion of the polynucleotide sequence in the comparison window may comprise additions, deletions, substitutions, and the like (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman, Adv. Appl. Math. 2:482 (1981), by the homology alignment algorithm of Needleman and Wunsch, J. Mol. Biol. 48:443 (1970), by the search for similarity method of Pearson and Lipman, Proc. Natl. Acad. Sci. (U.S.A.) 85:2444 (1988), by computerized implementations of these algorithms (GAP, BESTFIT, FASTA, and TFASTA in the Wisconsin Genetics Software Package Release 7.0, (Genetics Computer Group, 575 Science Dr., Madison, Wis.), Geneworks, or MacVector software packages), or by inspection, and the best alignment (i.e., resulting in the highest percentage of homology over the comparison window) generated by the various methods is selected.

The term “sequence identity” means that two polynucleotide or amino acid sequences are identical (i.e., on a nucleotide-by-nucleotide or residue-by-residue basis) over the comparison window. The term “percentage of sequence identity” is calculated by comparing two optimally aligned sequences over the window of comparison, determining the number of positions at which the identical nucleic acid base (e.g., A, T, C, G, U, or I) or residue occurs in both sequences to yield the number of matched positions, dividing the number of matched positions by the total number of positions in the comparison window (i.e., the window size), and multiplying the result by 100 to yield the percentage of sequence identity. The terms “substantial identity” as used herein denotes a characteristic of a polynucleotide or amino acid sequence, wherein the polynucleotide or amino acid comprises a sequence that has at least 85 percent sequence identity, preferably at least 90 to 95 percent sequence identity, more usually at least 99 percent sequence identity as compared to a reference sequence over a comparison window of at least 18 nucleotide (6 amino acid) positions, frequently over a window of at least 24-48 nucleotide (8-16 amino acid) positions, wherein the percentage of sequence identity is calculated by comparing the reference sequence to the sequence which may include deletions or additions which total 20 percent or less of the reference sequence over the comparison window. The reference sequence may be a subset of a larger sequence.

As used herein, the twenty conventional amino acids and their abbreviations follow conventional usage. See Immunology—A Synthesis (2d ed., Golub, E. S. and Gren, D. R. eds., Sinauer Associates, Sunderland, Mass. 1991), which is incorporated herein by reference. Stereoisomers (e.g., D-amino acids) of the twenty conventional amino acids, unnatural amino acids such as α-,α-disubstituted amino acids, N-alkyl amino acids, lactic acid, and other unconventional amino acids may also be suitable components for polypeptides of the invention described herein. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA and which are 5′ to the 5′ end of the RNA transcript are referred to as “upstream sequences”; sequence regions on the DNA strand having the same sequence as the RNA and which are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity, and most preferably at least 99 percent sequence identity. Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, glutamic-aspartic, and asparagine-glutamine.

As discussed herein, minor variations in the amino acid sequences of antibodies or immunoglobulin molecules are contemplated as being encompassed by the invention described herein, providing that the variations in the amino acid sequence maintain at least 75%, more preferably at least 80%, 90%, 95%, and most preferably 99%. In particular, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al., Science 253:164 (1991). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. Analogs can include various muteins of a sequence other than the naturally occurring peptide sequence. For example, single or multiple amino acid substitutions (preferably conservative amino acid substitutions) may be made in the naturally occurring sequence (preferably in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, ed., W. H. Freeman and Company, New York 1984); Introduction to Protein Structure (Branden, C. and Tooze, J. eds., Garland Publishing, New York, N.Y. 1991); and Thornton et al., Nature 354:105 (1991), which are each incorporated herein by reference.

The term “polypeptide fragment” as used herein refers to a polypeptide that has an amino-terminal and/or carboxy-terminal deletion, but where the remaining amino acid sequence is identical to the corresponding positions in the naturally occurring sequence deduced, for example, from a full-length cDNA sequence. Fragments typically are at least 5, 6, 8 or 10 amino acids long, preferably at least 14 amino acids long, more preferably at least 20 amino acids long, usually at least 50 amino acids long, and even more preferably at least 70 amino acids long. The term “analog” as used herein refers to polypeptides which are comprised of a segment of at least 25 amino acids that has substantial identity to a portion of a deduced amino acid sequence and which has at least one of the following properties: (1) specific binding to a PLA2, under suitable binding conditions, (2) ability to block appropriate PLA2 binding, or (3) ability to inhibit PLA2 expressing cell growth in vitro or in vivo. Typically, polypeptide analogs comprise a conservative amino acid substitution (or addition or deletion) with respect to the naturally occurring sequence. Analogs typically are at least 20 amino acids long, preferably at least 50 amino acids long or longer, and can often be as long as a full-length naturally occurring polypeptide.

Peptide analogs are commonly used in the pharmaceutical industry as non-peptide drugs with properties analogous to those of the template peptide. These types of non-peptide compound are termed “peptide mimetics” or “peptidomimetics.” Fauchere, J. Adv. Drug Res. 15:29 (1986); Veber and Freidinger, TINS p.392 (1985); and Evans et al., J. Med. Chem. 30:1229 (1987), which are incorporated herein by reference. Such compounds are often developed with the aid of computerized molecular modeling. Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from the group consisting of: —CH₂NH—, —CH₂S—, —CH₂—CH₂—, —CH═CH—(cis and trans), —COCH₂—, —CH(OH)CH₂—, and —CH₂SO—, by methods well known in the art. Systematic substitution of one or more amino acids of a consensus sequence with a D-amino acid of the same type (e.g., D-lysine in place of L-lysine) may be used to generate more stable peptides. In addition, constrained peptides comprising a consensus sequence or a substantially identical consensus sequence variation may be generated by methods known in the art (Rizo and Gierasch Ann. Rev. Biochem. 61:387 (1992), incorporated herein by reference); for example, by adding internal cysteine residues capable of forming intramolecular disulfide bridges which cyclize the peptide.

“Antibody” or “antibody peptide(s)” refer to an intact antibody, or a binding fragment thereof that competes with the intact antibody for specific binding. Binding fragments are produced by recombinant DNA techniques, or by enzymatic or chemical cleavage of intact antibodies. Binding fragments include Fab, Fab′, F(ab′)₂, Fv, and single-chain antibodies. An antibody other than a “bispecific” or “bifunctional” antibody is understood to have each of its binding sites identical. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay).

The term “epitope” includes any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An antibody is said to specifically bind an antigen when the dissociation constant is ≦1 μM, preferably ≦100 nM and most preferably ≦10 nM.

The term “agent” is used herein to denote a chemical compound, a mixture of chemical compounds, a biological macromolecule, or an extract made from biological materials.

“Active” or “activity” for the purposes herein refers to form(s) of PLA2 polypeptide which retain a biological and/or an immunological activity of native or naturally occurring PLA2 polypeptides, wherein “biological” activity refers to a biological function (either inhibitory or stimulatory) caused by a native or naturally occurring PLA2 polypeptide other than the ability to induce the production of an antibody against an antigenic epitope possessed by a native or naturally occurring PLA2 polypeptide and an “immunological” activity refers to the ability to induce the production of an antibody against an antigenic epitope possessed by a native or naturally occurring PLA2 polypeptide.

“Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

“Mammal” refers to any animal classified as a mammal, including humans, other primates, such as monkeys, chimpanzees and gorillas, domestic and farm animals, and zoo, sports, laboratory, or pet animals, such as dogs, cats, cattle, horses, sheep, pigs, goats, rabbits, rodents, etc. For purposes of treatment, the mammal is preferably human.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an “F(ab′)₂” fragment that has two antigen-combining sites and is still capable of cross-linking antigen.

“Fv” is the minimum antibody fragment that contains a complete antigen-recognition and binding site of the antibody. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, for example, even a single variable domain (e.g., the VH or VL portion of the Fv dimer or half of an Fv comprising only three CDRs specific for an antigen) may have the ability to recognize and bind antigen, although, possibly, at a lower affinity than the entire binding site.

A Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. F(ab′)₂ antibody fragments originally were produced as pairs of Fab′ fragments which have hinge cysteines between them. Other chemical couplings of antibody fragments are also known.

“Solid phase” means a non-aqueous matrix to which the antibodies described herein can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phases can comprise the well of an assay plate; in others it is a purification column (e.g., an affinity chromatography column). This term also includes a discontinuous solid phase of discrete particles, such as those described in U.S. Pat. No. 4,275,149.

The term “liposome” is used herein to denote a small vesicle composed of various types of lipids, phospholipids and/or surfactant which is useful for delivery of a drug (such as a PLA2 polypeptide or antibody thereto) to a mammal. The components of the liposomes are commonly arranged in a bilayer formation, similar to the lipid arrangement of biological membranes.

The term “small molecule” is used herein to describe a molecule with a molecular weight below about 500 Daltons.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

The term “pharmaceutical agent or drug” as used herein refers to a chemical compound or composition capable of inducing a desired therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985)), incorporated herein by reference).

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “patient” includes human and veterinary subjects.

Anti-PLA2 Antibodies

Antibodies, or parts, fragments, mimetics, or derivatives thereof, may be any type of antibody or part which recognizes a PLA2. In certain embodiments, it is preferred that the antibody, or part thereof, can neutralize PLA2. In additional embodiments it is preferred that the antibody, or part thereof, can reduce the symptoms associated with inflammatory conditions, including but not limited to inflammation, fluid retention, tissue swelling, pain, puffiness, high blood pressure, and brain swelling.

Antibody Structure

The basic antibody structural unit is known to comprise a tetramer. Each tetramer is composed of two identical pairs of polypeptide chains, each pair having one “light” (about 25 kDa) and one “heavy” chain (about 50 to 70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)) (incorporated by reference in its entirety for all purposes). The variable regions of each light/heavy chain pair form the antibody-binding site. Thus, an intact antibody has two binding sites. Except in bifunctional or bispecific antibodies, the two binding sites are the same.

The chains all exhibit the same general structure of relatively conserved framework regions (FR) joined by three hyper variable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md. 1991) (1987), or Chothia and Lesk, J. Mol. Biol. 196:901-17 (1987); Chothia et al., Nature 342:878-83 (1989).

A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai and Lachmann, Clin. Exp. Immunol. 79: 315-21 (1990); Kostelny et al., J. Immunol. 148:1547-53 (1992). Production of bispecific antibodies can be a relatively labor intensive process compared with production of conventional antibodies and yields and degree of purity are generally lower for bispecific antibodies. Bispecific antibodies do not exist in the form of fragments having a single binding site (e.g., Fab, Fab′, and Fv).

It will be appreciated that such bifunctional or bispecific antibodies are contemplated and encompassed by the invention.

Human Antibodies and Humanization of Antibodies

Embodiments of the invention described herein also contemplate and encompass human antibodies. For treatment of a human, human antibodies avoid certain of the problems associated with antibodies that possess murine or rat variable and/or constant regions. The presence of such murine or rat derived proteins can lead to the rapid clearance of the antibodies or can lead to the generation of an immune response against the antibody by a patient. In order to avoid the utilization of murine or rat derived antibodies, it has been postulated that one can develop humanized antibodies or generate fully human antibodies through the introduction of human antibody function into a rodent so that the rodent would produce fully human antibodies.

### Human Antibodies

One method for generating fully human antibodies is through the use of XenoMouse® strains of mice that have been engineered to contain human heavy chain and light chain genes within their genome. For example, a XenoMouse® mouse containing 245 kb and 190 kb-sized germline configuration fragments of the human heavy chain locus and kappa light chain locus is described in Green et al., Nature Genetics 7:13-21 (1994). The work of Green et al. was extended to the introduction of greater than approximately 80% of the human antibody repertoire through utilization of megabase-sized, germline configuration YAC fragments of the human heavy chain loci and kappa light chain loci, respectively. See Mendez et al., Nature Genetics 15:146-56 (1997) and U.S. patent application Ser. No. 08/759,620, filed Dec. 3, 1996, the disclosures of which are hereby incorporated by reference. Further, XenoMouse® mice have been generated that contain the entire lambda light chain locus (U.S. Patent Application Ser. No. 60/334,508, filed Nov. 30, 2001). And, XenoMouse® mice have been generated that produce multiple isotypes (see, e.g., WO 00/76310). XenoMouse® strains are available from Abgenix, Inc. (Fremont, Calif.).

The production of XenoMouse® mice is further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, filed Ser. No. 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430,938, Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996 and U.S. Pat. Nos. 6,162,963, 6,150,584, 6,114,598, 6,075,181, and 5,939,598 and Japanese Patent Nos. 3 068 180 B2, 3 068 506 B2, and 3 068 507 B2. See also Mendez et al. Nature Genetics 15:146-156 (1997) and Green and Jakobovits J. Exp. Med., 188:483-495 (1998). See also European Patent No., EP 463,151 B1, grant published Jun. 12, 1996, International Patent Application No., WO 94/02602, published Feb. 3, 1994, International Patent Application No., WO 96/34096, published Oct. 31, 1996, WO 98/24893, published Jun. 11, 1998, WO 00/76310, published Dec. 21, 2000. The disclosures of each of the above-cited patents, applications, and references are hereby incorporated by reference in their entirety.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more V_{H} genes, one or more D_{H} genes, one or more J_{H} genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806, 5,625,825, 5,625,126, 5,633,425, 5,661,016, 5,770,429, 5,789,650, 5,814,318, 5,877,397, 5,874,299, and 6,255,458 each to Lonberg and Kay, U.S. Pat. Nos. 5,591,669 and 6,023.010 to Krimpenfort and Berns, U.S. Pat. Nos. 5,612,205, 5,721,367, and 5,789,215 to Berns et al., and U.S. Pat. No. 5,643,763 to Choi and Dunn, and GenPharm International U.S. patent application Ser. No. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed Mar. 9, 1994, the disclosures of which are hereby incorporated by reference. See also European Patent No. 546,073 B1, International Patent Application Nos. WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175, the disclosures of which are hereby incorporated by reference in their entirety. See further Taylor et al., (1992), Chen et al., (1993), Tuaillon et al., (1993), Choi et al., (1993), Lonberg et al., (1994), Taylor et al., (1994), and Tuaillon et al., (1995), Fishwild et al., (1996), the disclosures of which are hereby incorporated by reference in their entirety.

Kirin has demonstrated the generation of human antibodies from mice in which, through microcell fusion, large pieces of chromosomes, or entire chromosomes, have been introduced. See European Patent Application Nos. 773,288 and 843,961, the disclosures of which are hereby incorporated by reference.

Lidak Pharmaceuticals (now Xenorex) has also demonstrated the generation of human antibodies in SCID mice modified by injection of non-malignant mature peripheral leukocytes from a human donor. The modified mice exhibit an immune response characteristic of the human donor upon stimulation with an immunogen, which consists of the production of human antibodies. See U.S. Pat. Nos. 5,476,996 and 5,698,767, the disclosures of which are herein incorporated by reference.

Human anti-mouse antibody (HAMA) responses have led the industry to prepare chimeric or otherwise humanized antibodies. While chimeric antibodies have a human constant region and a murine variable region, it is expected that certain human anti-chimeric antibody (HACA) responses will be observed, particularly in chronic or multi-dose utilizations of the antibody. Thus, it would be desirable to provide fully human antibodies against PLA2 in order to vitiate concerns and/or effects of HAMA or HACA response.

### Humanization and Display Technologies

As discussed above in connection with human antibody generation, there are advantages to producing antibodies with reduced immunogenicity. To a degree, this can be accomplished in connection with techniques of humanization and display techniques using appropriate libraries. It will be appreciated that murine antibodies or antibodies from other species can be humanized or primatized using techniques well known in the art. See e.g., Winter and Harris, Immunol Today 14:43-46 (1993) and Wright et al., Crit, Reviews in Immunol. 12:125-168 (1992). The antibody of interest may be engineered by recombinant DNA techniques to substitute the CH1, CH2, CH3, hinge domains, and/or the framework domain with the corresponding human sequence (see WO 92/02190 and U.S. Pat. Nos. 5,530,101, 5,585,089, 5,693,761, 5,693,792, 5,714,350, and 5,777,085). Also, the use of Ig cDNA for construction of chimeric immunoglobulin genes is known in the art (Liu et al., P.N.A.S. 84:3439 (1987) and J. Immunol. 139:3521 (1987)). mRNA is isolated from a hybridoma or other cell producing the antibody and used to produce cDNA. The cDNA of interest may be amplified by the polymerase chain reaction using specific primers (U.S. Pat. Nos. 4,683,195 and 4,683,202). Alternatively, a library can be made and screened to isolate the sequence of interest. The DNA sequence encoding the variable region of the antibody is then fused to human constant region sequences. The sequences of human constant regions genes may be found in Kabat et al., “Sequences of Proteins of Immunological Interest,” N.I.H. publication no. 91-3242 (1991). Human C region genes are readily available from known clones. The choice of isotype will be guided by the desired effector functions, such as complement fixation, or activity in antibody-dependent cellular cytotoxicity. Preferred isotypes are IgG1, IgG3 and IgG4. Either of the human light chain constant regions, kappa or lambda, may be used. The chimeric, humanized antibody is then expressed by conventional methods.

Antibody fragments, such as Fv, F(ab′)₂ and Fab may be prepared by cleavage of the intact protein, e.g., by protease or chemical cleavage. Alternatively, a truncated gene is designed. For example, a chimeric gene encoding a portion of the F(ab′)₂ fragment would include DNA sequences encoding the CH1 domain and hinge region of the H chain, followed by a translational stop codon to yield the truncated molecule.

Consensus sequences of heavy and light J regions may be used to design oligonucleotides for use as primers to introduce useful restriction sites into the J region for subsequent linkage of V region segments to human C region segments. C region cDNA can be modified by site directed mutagenesis to place a restriction site at the analogous position in the human sequence.

Expression vectors include plasmids, retroviruses, YACs, EBV derived episomes, and the like. A convenient vector is one that encodes a functionally complete human CH or CL immunoglobulin sequence, with appropriate restriction sites engineered so that any VH or VL sequence can be easily inserted and expressed. In such vectors, splicing usually occurs between the splice donor site in the inserted J region and the splice acceptor site preceding the human C region, and also at the splice regions that occur within the human CH exons. Polyadenylation and transcription termination occur at native chromosomal sites downstream of the coding regions. The resulting chimeric antibody may be joined to any strong promoter, including retroviral LTRs, e.g., SV-40 early promoter, (Okayama et al., Mol. Cell. Bio. 3:280 (1983)), Rous sarcoma virus LTR (Gorman et al., P.N.A.S. 79:6777 (1982)), and moloney murine leukemia virus LTR (Grosschedl et al., Cell 41:885 (1985)). Also, as will be appreciated, native Ig promoters and the like may be used.

Further, human antibodies or antibodies from other species can be generated through display-type technologies, including, without limitation, phage display, retroviral display, ribosomal display, and other techniques, using techniques well known in the art and the resulting molecules can be subjected to additional maturation, such as affinity maturation, as such techniques are well known in the art. Wright and Harris, supra., Hanes and Plucthau, PNAS USA 94:4937-4942 (1997) (ribosomal display), Parmley and Smith, Gene 73:305-318 (1988) (phage display), Scott, TIBS 17:241-245 (1992), Cwirla et al., PNAS USA 87:6378-6382 (1990), Russel et al., Nucl. Acids Res. 21:1081-1085 (1993), Hoganboom et al., Immunol. Reviews 130:43-68 (1992), Chiswell and McCafferty, TIBTECH 10:80-84 (1992), and U.S. Pat. No. 5,733,743. If display technologies are utilized to produce antibodies that are not human, such antibodies can be humanized as described above.

Using these techniques, antibodies can be generated against PLA2 expressing cells, PLA2 itself, forms of PLA2, epitopes or peptides thereof, and expression libraries thereto (see e.g. U.S. Pat. No. 5,703,057) which can thereafter be screened as described above for the activities described above.

Preparation of Antibodies

Through use of XenoMouse® technology, fully human monoclonal antibodies specific for the PLA2 were produced. Essentially, XenoMouse® lines of mice were immunized with PLA2; or fragments thereof, lymphatic cells (such as B-cells) were recovered from the mice that express antibodies, recovered cells were fused with a myeloid-type cell line to prepare immortal hybridoma cell lines, and such hybridoma cell lines were screened and selected to identify hybridoma cell lines that produced antibodies specific to PLA2. Further, a characterization of the antibodies produced by such cell lines is described herein, including nucleotide and amino acid sequence analyses of the heavy and light chains of such antibodies.

Alternatively, instead of being fused to myeloma cells to generate hybridomas, the recovered cells, isolated from immunized XenoMouse® lines of mice, are screened further for reactivity against the initial antigen, preferably PLA2 protein. Such screening includes Enzyme-Linked Immunosorbent Assay (ELISA) with PLA2-His protein, a competition assay with known antibodies that bind the antigen of interest, and in vitro binding to transiently transfected CHO cells expressing full length PLA2. Single B cells secreting antibodies of interest are then isolated using a PLA2-specific hemolytic plaque assay (Babcook et al., Proc. Natl. Acad. Sci. USA, i93:7843-7848 (1996)). Cells targeted for lysis are preferably sheep red blood cells (SRBCs) coated with the PLA2 antigen. In the presence of a B cell culture secreting the immunoglobulin of interest and complement, the formation of a plaque indicates specific PLA2-mediated lysis of the target cells. The single antigen-specific plasma cell in the center of the plaque can be isolated and the genetic information that encodes the specificity of the antibody is isolated from the single plasma cell. Using reverse-transcriptase PCR, the DNA encoding the variable region of the antibody secreted can be cloned. Such cloned DNA can then be further inserted into a suitable expression vector, preferably a vector cassette such as a pcDNA, more preferably such a pcDNA vector containing the constant domains of immunglobulin heavy and light chain. The generated vector can then be transfected into host cells, preferably CHO cells, and cultured in conventional nutrient media modified as appropriate for inducing promoters, selecting transformants, or amplifying the genes encoding the desired sequences. Further, the genetic material that encodes the specificity of the anti-PLA2 antibody can be isolated, introduced into a suitable expression vector which is then transfected into host cells.

In general, antibodies produced by the above-mentioned cell lines possessed either fully human IgG2 heavy chains with human kappa light chains or fully human IgG4 heavy chains with human kappa light chains. The antibodies possessed high affinities, typically possessing Kd's of from about 10⁻⁶ through about 10⁻¹¹ M, when measured by either solid phase and solution phase.

Regarding the importance of affinity to therapeutic utility of anti-PLA2 antibodies, it will be understood that one can generate anti-PLA2 antibodies, for example, combinatorially, and assess such antibodies for binding affinity. One approach that can be utilized is to take the heavy chain cDNA from an antibody, prepared as described above and found to have good affinity to PLA2, and combine it with the light chain cDNA from a second antibody, prepared as described above and also found to have good affinity to PLA2, to produce a third antibody. The affinities of the resulting third antibodies can be measured as described herein and those with desirable dissociation constants isolated and characterized. Alternatively, the light chain of any of the antibodies described above can be used as a tool to aid in the generation of a heavy chain that when paired with the light chain will exhibit a high affinity for PLA2, or vice versa. These heavy chain variable regions in this library could be isolated from naïve animals, isolated from hyperimmune animals, generated artificially from libraries containing variable heavy chain sequences that differ in the CDR regions, or generated by any other methods that produce diversity within the CDR regions of any heavy chain variable region gene (such as random or directed mutagenesis). These CDR regions, and in particular CDR3, may be a significantly different length or sequence identity from the heavy chain initially paired with the original antibody. The resulting library could then be screened for high affinity binding to PLA2 to generate a therapeutically relevant antibody molecule with similar properties as the original antibody (high affinity and neutralization). A similar process using the heavy chain or the heavy chain variable region can be used to generate a therapeutically relevant antibody molecule with a unique light chain variable region. Furthermore, the novel heavy chain variable region, or light chain variable region, can then be used in a similar fashion as described above to identify a novel light chain variable region, or heavy chain variable region, that allows the generation of a novel antibody molecule.

Another combinatorial approach that can be utilized is to perform mutagenesis on germ line heavy and/or light chains that are demonstrated to be utilized in the antibodies in accordance with the invention described herein, particularly in the complementarity determining regions (CDRs). The affinities of the resulting antibodies can be measured as described herein and those antibodies with desirable dissociation constants isolated and characterized. Upon selection of a preferred binder, the sequence or sequences encoding the same may be used to generate recombinant antibodies as described above. Appropriate methods of performing mutagenesis on an oligonucleotide are known to those skilled in the art and include chemical mutagenesis, for example, with sodium bisulfite, enzymatic misincorporation, and exposure to radiation. It is understood that the invention described herein encompasses antibodies with substantial identity, as defined herein, to the antibodies explicitly set forth herein, whether produced by mutagenesis or by any other means. Further, antibodies with conservative or non-conservative amino acid substitutions, as defined herein, made in the antibodies explicitly set forth herein, are included in embodiments of the invention described herein.

Another combinatorial approach that can be used is to express the CDR regions, and in particular CDR3, of the antibodies described above in the context of framework regions derived from other variable region genes. For example, CDR1, CDR2, and CDR3 of the heavy chain of one anti-PLA2 antibody could be expressed in the context of the framework regions of other heavy chain variable genes. Similarly, CDR1, CDR2, and CDR3 of the light chain of an anti-PLA2 antibody could be expressed in the context of the framework regions of other light chain variable genes. In addition, the germline sequences of these CDR regions could be expressed in the context of other heavy or light chain variable region genes. The resulting antibodies can be assayed for specificity and affinity and may allow the generation of a novel antibody molecule.

As will be appreciated, antibodies in accordance with the embodiments of the invention can be expressed in cell lines other than hybridoma cell lines. Sequences encoding particular antibodies can be used for transformation of a suitable mammalian host cell. Transformation can be by any known method for introducing polynucleotides into a host cell, including, for example packaging the polynucleotide in a virus (or into a viral vector) and transducing a host cell with the virus (or vector) or by transfection procedures known in the art, as exemplified by U.S. Pat. Nos. 4,399,216, 4,912,040, 4,740,461, and 4,959,455 (which patents are hereby incorporated herein by reference). The transformation procedure used depends upon the host to be transformed. Methods for introduction of heterologous polynucleotides into mammalian cells are well known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei.

Mammalian cell lines available as hosts for expression are well known in the art and include many immortalized cell lines available from the American Type Culture Collection (ATCC), including but not limited to Chinese hamster ovary (CHO) cells, HeLa cells, baby hamster kidney (BHK) cells, monkey kidney cells (COS), human hepatocellular carcinoma cells (e.g., Hep G2), and a number of other cell lines. Cell lines of particular preference are selected through determining which cell lines have high expression levels and produce antibodies with constitutive PLA2 binding properties.

Antibodies in accordance with the embodiments of the invention are capable of binding to PLA2. Further, antibodies of the invention are useful in the detection of PLA2 in patient samples and accordingly are useful as diagnostics as described hereinbelow. In addition, based on the known relationship of PLA2 to inflammation, it is expected that such antibodies will have therapeutic effect in the treatment of inflammation.

Additional Criteria for Antibody Therapeutics

As discussed herein, the function of the PLA2 antibody appears important to at least a portion of its mode of operation. By function, is meant, by way of example, the activity of the PLA2 antibody in response to a PLA2 antigen. Accordingly, in certain respects, it may be desirable in connection with the generation of antibodies as therapeutic candidates against PLA2 that the antibodies may be made capable of effector function, including complement-dependent cytotoxicity (CDC) and antibody-dependent cellular cytotoxicity (ADCC). There are a number of isotypes of antibodies that are capable of the same, including, without limitation, the following: murine IgM, murine IgG2a, murine IgG2b, murine IgG3, human IgM, human IgG1, human IgG3, and human IgG4. It will be appreciated that antibodies that are generated need not initially possess such an isotype but, rather, the antibody as generated can possess any isotype and the antibody can be isotype switched thereafter using conventional techniques that are well known in the art. Such techniques include the use of direct recombinant techniques (see, e.g., U.S. Pat. No. 4,816,397), cell-cell fusion techniques (see, e.g., U.S. Pat. Nos. 5,916,771 and 6,207,418), among others.

In the cell-cell fusion technique, a myeloma or other cell line is prepared that possesses a heavy chain with any desired isotype and another myeloma or other cell line is prepared that possesses the light chain. Such cells can, thereafter, be fused and a cell line expressing an intact antibody can be isolated.

By way of example, one type of PLA2 antibody discussed herein is a human anti-PLA2 IgG2 antibody. If such antibody possessed desired binding to the PLA2 molecule, it could be readily isotype switched to generate a human IgM, human IgG1, human IgG3, or human IgG4 isotype, while still possessing the same variable region (which defines the antibody's specificity and some of its affinity). Such molecule would then be capable of fixing complement and participating in CDC.

Accordingly, as antibody candidates are generated that meet desired “structural” attributes as discussed above, they can generally be provided with at least certain of the desired “functional” attributes through isotype switching.

Epitope Mapping

### Immunoblot Analysis

The binding of the antibodies described herein to PLA2 can be examined by a number of methods. For example, PLA2 may be subjected to SDS-PAGE and analyzed by immunoblotting. The SDS-PAGE may be performed either in the absence or presence of a reduction agent. Such chemical modifications may result in the methylation of cysteine residues. Accordingly, it is possible to determine whether the PLA2 antibodies described herein bind to a linear epitope on PLA2.

### Surface-Enhanced Laser Desorption/Ionization

Epitope mapping of the epitope for the PLA2 antibodies described herein can also be performed using SELDI. SELDI ProteinChip® arrays are used to define sites of protein-protein interaction. Antigens are specifically captured on antibodies covalently immobilized onto the Protein Chip array surface by an initial incubation and wash. The bound antigens can be detected by a laser-induced desorption process and analyzed directly to determine their mass. Such fragments of the antigen that bind are designated as the “epitope” of a protein.

The SELDI process enables individual components within complex molecular compositions to be detected directly and mapped quantitatively relative to other components in a rapid, highly-sensitive and scalable manner. SELDI utilizes a diverse array of surface chemistries to capture and present large numbers of individual protein molecules for detection by a laser-induced desorption process. The success of the SELDI process is defined in part by the miniaturization and integration of multiple functions, each dependent on different technologies, on a surface (“chip”). SELDI BioChips and other types of SELDI probes are surfaces “enhanced” such that they become active participants in the capture, purification (separation), presentation, detection, and characterization of individual target molecules (e.g. proteins) or population of molecules to be evaluated.

A single SELDI protein BioChip, loaded with only the original sample, can be read thousands of times. The SELDI protein BioChips from LumiCyte hold as many as 10,000 addressable protein docking locations per 1 square centimeter. Each location may reveal the presence of dozens of individual proteins. When the protein composition information from each location is compared and unique information sets combined, the resulting composition map reveals an image with sets of features that are used collectively to define specific patterns or molecular “fingerprints.” Different fingerprints may be associated with various stages of health, the onset of disease, or the regression of disease associated with the administration of appropriate therapeutics.

The SELDI process may be described in further detail in four parts. Initially, one or more proteins of interest are captured or “docked” on the ProteinChip Array, directly from the original source material, without sample preparation and without sample labeling. In a second step, the “signal-to-noise” ratio is enhanced by reducing the chemical and biomolecular “noise.” Such “noise” is reduced through selective retention of target on the chip by washing away undesired materials. Further, one or more of the target protein(s) that are captured are read by a rapid, sensitive, laser-induced process (SELDI) that provides direct information about the target (molecular weight). Lastly, the target protein at any one or more locations within the array may be characterized in situ by performing one or more on-the-chip binding or modification reactions to characterize protein structure and function.

### Phage Display

The epitope for the PLA2 antibodies described herein can be determined by exposing the ProteinChip Array to a combinatorial library of random peptide 12-mer displayed on Filamentous phage (New England Biolabs).

Phage display describes a selection technique in which a peptide is expressed as a fusion with a coat protein of a bacteriophage, resulting in display of the fused protein on the surface of the virion. Panning is carried out by incubation of a library of phage displayed peptide with a plate or tube coated with the target, washing away the unbound phage, and eluting the specifically bound phage. The eluted phage is then amplified and taken through additional binding and amplification cycles to enrich the pool in favor of binding sequences. After three or four rounds, individual clones binding are further tested for binding by phage ELISA assays performed on antibody-coated wells and characterized by specific DNA sequencing of positive clones.

After multiple rounds of such panning against the PLA2 antibodies described herein, the bound phage may be eluted and subjected to further studies for the identification and characterization of the bound peptide.

PLA2 Agonists and Antagonists

Embodiments of the invention described herein also pertain to variants of a PLA2 protein that function as either PLA2 agonists (mimetics) or as PLA2 antagonists. Variants of a PLA2 protein can be generated by mutagenesis, e.g., discrete point mutation or truncation of the PLA2 protein. An agonist of the PLA2 protein can retain substantially the same, or a subset of, the biological activities of the naturally occurring form of the PLA2 protein. An antagonist of the PLA2 protein can inhibit one or more of the activities of the naturally occurring form of the PLA2 protein by, for example, competitively binding to a downstream or upstream member of a cellular signaling cascade which includes the PLA2 protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the PLA2 protein.

Variants of the PLA2 protein that function as either PLA2 agonists (mimetics) or as PLA2 antagonists can be identified by screening combinatorial libraries of mutants, e.g., truncation mutants, of the PLA2 protein for protein agonist or antagonist activity. In one embodiment, a variegated library of PLA2 variants is generated by combinatorial mutagenesis at the nucleic acid level and is encoded by a variegated gene library. A variegated library of PLA2 variants can be produced by, for example, enzymatically ligating a mixture of synthetic oligonucleotides into gene sequences such that a degenerate set of potential PLA2 sequences is expressible as individual polypeptides, or alternatively, as a set of larger fusion proteins (e.g., for phage display) containing the set of PLA2 sequences therein. There are a variety of methods which can be used to produce libraries of potential PLA2 variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential PLA2 variant sequences. Methods for synthesizing degenerate oligonucleotides are known in the art (see, e.g., Narang, Tetrahedron 39:3 (1983); Itakura et al., Annu. Rev. Biochem. 53:323 (1984); Itakura et al., Science 198:1056 (1984); Ike et al., Nucl. Acid Res. 11:477 (1983).

Design and Generation of Other Therapeutics

Moreover, based on the activity of the antibodies that are produced and characterized herein with respect to PLA2, the design of other therapeutic modalities beyond antibody moieties is facilitated. Such modalities include, without limitation, advanced antibody therapeutics, such as bispecific antibodies, immunotoxins, and radiolabeled therapeutics, generation of peptide therapeutics, gene therapies, particularly intrabodies, antisense therapeutics, and small molecules.

In connection with the generation of advanced antibody therapeutics, where complement fixation is a desirable attribute, it may be possible to sidestep the dependence on complement for cell killing through the use of bispecifics, immunotoxins, or radiolabels, for example.

For example, bispecific antibodies can be generated that comprise (i) two antibodies one with a specificity to PLA2 and another to a second molecule that are conjugated together, (ii) a single antibody that has one chain specific to PLA2 and a second chain specific to a second molecule, or (iii) a single chain antibody that has specificity to PLA2 and the other molecule. Such bispecific antibodies can be generated using techniques that are well known for example, in connection with (i) and (ii) see e.g., Fanger et al. Immunol Methods 4:72-81 (1994) and Wright and Harris, supra. and in connection with (iii) see e.g., Traunecker et al. Int. J. Cancer (Suppl.) 7:51-52 (1992). In each case, the second specificity can be made to the heavy chain activation receptors, including, without limitation, CD16 or CD64 (see e.g., Deo et al. 18:127 (1997)) or CD89 (see e.g., Valerius et al. Blood 90:4485-4492 (1997)). Bispecific antibodies prepared in accordance with the foregoing would be likely to kill cells expressing PLA2, and particularly those cells in which the PLA2 antibodies of the invention are effective.

In connection with immunotoxins, antibodies can be modified to act as immunotoxins utilizing techniques that are well known in the art. See e.g., Vitetta Immunol Today 14:252 (1993). See also U.S. Pat. No. 5,194,594. In connection with the preparation of radiolabeled antibodies, such modified antibodies can also be readily prepared utilizing techniques that are well known in the art. See e.g., Junghans et al. in Cancer Chemotherapy and Biotherapy 655-686 (2d edition, Chafner and Longo, eds., Lippincott Raven (1996)). See also U.S. Pat. Nos. 4,681,581, 4,735,210, 5,101,827, 5,102,990 (RE 35,500), 5,648,471, and 5,697,902. Each of immunotoxins and radiolabeled molecules would be likely to kill cells expressing PLA2, and particularly those cells in which the antibodies of the invention are effective.

In connection with the generation of therapeutic peptides, through the utilization of structural information related to PLA2 and antibodies thereto, such as the antibodies described herein (as discussed below in connection with small molecules) or screening of peptide libraries, therapeutic peptides can be generated that are directed against PLA2. Design and screening of peptide therapeutics is discussed in connection with Houghten et al., Biotechniques 13:412-421 (1992), Houghten, PNAS USA 82:5131-5135 (1985), Pinalla et al., Biotechniques 13:901-905 (1992), Blake and Litzi-Davis, BioConjugate Chem. 3:510-513 (1992). Immunotoxins and radiolabeled molecules can also be prepared, and in a similar manner, in connection with peptidic moieties as discussed above in connection with antibodies.

Assuming that the PLA2 molecule (or a form, such as a splice variant or alternate form) is functionally active in a disease process, it will also be possible to design gene and antisense therapeutics thereto through conventional techniques. Such modalities can be utilized for modulating the function of PLA2. In connection therewith the antibodies, as described herein, facilitate design and use of functional assays related thereto. A design and strategy for antisense therapeutics is discussed in detail in International Patent Application No. WO 94/29444. Design and strategies for gene therapy are well known. However, in particular, the use of gene therapeutic techniques involving intrabodies could prove to be particularly advantageous. See, e.g., Chen et al., Human Gene Therapy 5:595-601 (1994) and Marasco, Gene Therapy 4:11-15 (1997). General design of and considerations related to gene therapeutics is also discussed in International Patent Application No.: WO 97/38137.

Small molecule therapeutics can also be envisioned. Drugs can be designed to modulate the activity of PLA2, as described herein. Knowledge gleaned from the structure of the PLA2 molecule and its interactions with other molecules, as described herein, such as the antibodies described herein, and others can be utilized to rationally design additional therapeutic modalities. In this regard, rational drug design techniques such as X-ray crystallography, computer-aided (or assisted) molecular modeling (CAMM), quantitative or qualitative structure-activity relationship (QSAR), and similar technologies can be utilized to focus drug discovery efforts. Rational design allows prediction of protein or synthetic structures which can interact with the molecule or specific forms thereof which can be used to modify or modulate the activity of PLA2. Such structures can be synthesized chemically or expressed in biological systems. This approach has been reviewed in Capsey et al., Genetically Engineered Human Therapeutic Drugs (Stockton Press, NY (1988)). Further, combinatorial libraries can be designed and synthesized and used in screening programs, such as high throughput screening efforts.

Therapeutic Administration and Formulations

The anti-PLA2 compounds including, but not limited to, antibodies and fragments thereof are suitable for incorporation into pharmaceuticals that treat organisms in need of a compound that modulates PLA2. These pharmacologically active compounds can be processed in accordance with conventional methods of galenic pharmacy to produce medicinal agents for administration to organisms, e.g., animals and mammals including humans. In certain embodiments, the active ingredients can be incorporated into a pharmaceutical product with or without modification. Additional embodiments include the manufacture of pharmaceuticals or therapeutic agents that deliver the pharmacologically active compounds, described herein, by several routes. For example, and not by way of limitation, DNA, RNA, and viral vectors having sequence encoding the antibodies or fragments thereof can be used in certain embodiments. Additionally, nucleic acids encoding antibodies or fragments thereof can be administered alone or in combination with other active ingredients.

It will be appreciated that administration of therapeutic entities described herein can be administered in admixture with suitable carriers, excipients, stabilizers, and other agents that are incorporated into formulations to provide improved transfer, delivery, tolerance, and the like. Pharmaceutically acceptable carriers include organic or inorganic carrier substances suitable for parenteral, enteral (for example, oral) or topical application that do not deleteriously react with the pharmacologically active ingredients of this invention. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. Additional carriers, excipients, and stabilizers include buffers such as TRIS HCl, phosphate, citrate, acetate and other organic acid salts; antioxidants such as ascorbic acid; low molecular weight (less than about ten residues) peptides such as polyarginine, proteins, such as serum albumin, gelatin, or immunoglobulins; hydrophilic polymers such as polyvinylpyrrolidinone; amino acids such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium and/or nonionic surfactants such as TWEEN, PLURONICS or polyethyleneglycol. Many more suitable vehicles are described in Remmington's Pharmaceutical Sciences, 15th Edition, Easton:Mack Publishing Company, pages 1405-1412 and 1461-1487(1975) and The National Formulary XIV, 14th Edition, Washington, American Pharmaceutical Association (1975), herein incorporated by reference.

The route of antibody administration can be in accord with known methods, including, for example, but are not limited to, topical, transdermal, parenteral, gastrointestinal, transbronchial, and transalveolar. Parenteral routes of administration include, but are not limited to, electrical or direct injection or infusion such as direct injection into a central venous line, intravenous, intracerebral, intramuscular, intraperitoneal, intradermal, intraarterial, intrathecal, or intralesional routes. The antibody is preferably administered continuously by infusion, by bolus injection, or by sustained release systems as noted below. In a preferred embodiment the administration route can be subcutaneous injection. In an alternative embodiment, the antibodies are administered via the renal artery. Gastrointestinal routes of administration include, but are not limited to, ingestion and rectal. Transbronchial and transalveolar routes of administration include, but are not limited to, inhalation, either via the mouth or intranasally.

When used for in vivo administration, the antibody formulation is preferably sterile. This can be readily accomplished by filtration through sterile filtration membranes, prior to or following lyophilization and reconstitution. The antibody ordinarily will be stored in lyophilized form or in solution. In addition, the therapeutic composition can be pyrogen-free and in a parenterally acceptable solution having due regard for pH, isotonicity, and stability. Therapeutic antibody compositions generally are placed into a container having a sterile access port, for example, an intravenous solution bag or vial having a stopper pierceable by a hypodermic injection needle.

Sterile compositions for injection can be formulated according to conventional pharmaceutical practice as described in Remington's Pharmaceutical Sciences (18^{th} ed., Mack Publishing Company, Easton, Pa. (1990)). The pharmaceutical preparations can be sterilized and if desired mixed with auxiliary agents, for example, lubricants, preservatives, stabilizers, wetting agents, emulsifiers, salts for influencing osmotic pressure, buffers, antioxidants, coloring, flavoring and/or aromatic substances and the like that do not deleteriously react with the active compounds. For example, dissolution or suspension of the active compound in a vehicle such as water or naturally occurring vegetable oil like sesame, peanut, or cottonseed oil or a synthetic fatty vehicle like ethyl oleate or the like may be desired.

Suitable compositions having the pharmacologically active compounds of this invention that are suitable for parenteral administration include, but are not limited to, pharmaceutically acceptable sterile isotonic solutions. Such solutions include, but are not limited to, saline and phosphate buffered saline for injection into a central venous line, intravenous, intramuscular, intraperitoneal, intradermal, or subcutaneous injection.

Compositions having the pharmacologically active compounds of this invention that are suitable for gastrointestinal administration include, but not limited to, pharmaceutically acceptable powders, pills or liquids for ingestion and suppositories for rectal administration.

Suitable examples of sustained-release preparations include semipermeable matrices of solid hydrophobic polymers containing the polypeptide, which matrices are in the form of shaped articles, films or microcapsules. Examples of sustained-release matrices include polyesters, hydrogels (e.g., poly(2-hydroxyethyl-methacrylate) as described by Langer et al., J. Biomed Mater. Res., 15:167-277 (1981) and Langer, Chem. Tech., 12:98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22:547-556 (1983)), non-degradable ethylene-vinyl acetate (Langer et al., supra), degradable lactic acid-glycolic acid copolymers such as the LUPRON Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through disulfide interchange, stabilization may be achieved by modifying sulfhydryl residues, lyophilizing from acidic solutions, controlling moisture content, using appropriate additives, and developing specific polymer matrix compositions.

Sustained-release compositions also include liposomally entrapped antibodies of the invention. Liposomes containing such antibodies are prepared by methods known per se: U.S. Pat. No. DE 3,218,121; Epstein et al., Proc. Natl. Acad. Sci. USA, 82:3688-3692 (1985); Hwang et al., Proc. Natl. Acad. Sci. USA, 77:4030-4034 (1980); EP 52,322; EP 36,676; EP 88,046; EP 143,949; 142,641; Japanese patent application 83-118008; U.S. Pat. Nos. 4,485,045 and 4,544,545; and EP 102,324.

An effective amount of antibody to be employed therapeutically will depend, for example, upon the therapeutic objectives, the route of administration, and the condition of the patient. The dosage of the antibody will be determined by the attending physician taking into consideration various factors known to modify the action of drugs including severity and type of disease, body weight, sex, diet, time and route of administration, other medications and other relevant clinical factors. Accordingly, it will be necessary for the therapist to titer the dosage and modify the route of administration as required to obtain the optimal therapeutic effect. Typically, the clinician will administer antibody until a dosage is reached that achieves the desired effect. Therapeutically effective dosages may be determined by either in vitro or in vivo methods. The progress of this therapy is easily monitored by conventional assays or by the assays described herein.

Therapeutic efficacy and toxicity of such compounds can be determined by standard pharmaceutical procedures in cell cultures or experimental animals, for example, ED50 (the dose therapeutically effective in 50% of the population). Data obtained from treating an animal model or an alternative model may be used in formulating a range of dosage for use with other organisms, including humans. The dosage of such compounds lies preferably within a range of circulating concentrations that include the ED50 with no toxicity. The dosage varies within this range depending upon type of evectin, hybrid, binding partner, or fragment thereof, the dosage form employed, sensitivity of the organism, and the route of administration.

Normal dosage concentrations of various antibodies or fragments thereof can vary from approximately 0.1 to 100 mg/kg. Desirable dosage concentrations include, for example, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, and 100 mg/kg or more. One preferred dosage is 1 to 10 mg/kg.

In some embodiments, the dose of antibodies or fragments thereof produces a tissue or blood concentration or both from approximately 0.1 μM to 500 mM, preferably about 1 to 800 μM, and more preferably greater than about 10 μM to about 500 μM. Preferable doses are, for example, the amount required to achieve a tissue or blood concentration or both of 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 110 μM, 120 μM, 130 μM, 140 μM, 145 μM, 150 μM, 160 μM, 170 μM, 180 μM, 190 μM, 200 μM, 220 μM, 240 μM, 250 μM, 260 μM, 280 μM, 300 μM, 320 μM, 340 μM, 360 μM, 380 μM, 400 μM, 420 μM, 440 μM, 460 μM, 480 μM, and 500 μM. In alternative embodiments, doses that produce a tissue concentration of greater than 800 μM are can be used. A constant infusion of the antibodies, hybrids, binding partners, or fragments thereof can also be provided so as to maintain a stable concentration in the tissues as measured by blood levels.

Dosage and administration can be adjusted to provide sufficient levels of the active moiety or to maintain the desired effect. Embodiments herein include both short acting and long acting pharmaceutical compositions. Accordingly, embodiments include schedules where pharmaceutical compositions are administered approximately every 1, 2, 3, 4, 5, or 6 days, every week, once every 2 weeks, once every 3 weeks, once every 4 weeks, once every 5 weeks, once every 6 weeks, once every 7 weeks, or once every 8 weeks. Depending on half-life and clearance rate of the particular formulation, the pharmaceutical compositions described herein can be administered about once, twice, three, four, five, six, seven, eight, nine, and ten or more times per day.

Additional therapeutics may be administered in combination with, before, or after administration of the anti-PLA2 antibodies. These therapeutics may be used to treat symptoms of the disease or may decrease the side effects of the anti-PLA2 antibodies. They may also be used to enhance the activity of the anti-PLA2 antibodies. Any type of therapeutic may be used including, but not limited to, for example, antibiotics, diuretics, anesthetics, analgesics, anti-inflammatories, and insulin. Examples of agents that are typically used to treat inflammation and may be used in combination with the antibodies include steroidal anti-inflammatories, such as cortisone, and non-steroidal anti-inflammatory medications, such as acetaminophen, aspirin, ibuprofen, and naproxen, and the like.

Diagnostic Use

Embodiments of the invention are also useful for assays, particularly in vitro diagnostic assays, for example, for use in determining the level of PLA2 in patient samples. Such assays may be useful for diagnosing diseases associated with over expression of PLA2. In some embodiments, the disease is an inflammatory condition. The patient samples can be, for example, bodily fluids, preferably blood, more preferably blood serum, synoival fluid, tissue lysates, and extracts prepared from diseased tissues. Other embodiments of the invention are useful for diagnosing and staging inflammatory conditions and related diseases. Monitoring the level of PLA2 may be used as a surrogate measure of patient response to treatment and as a method of monitoring the severity of the disease in a patient. Elevated levels of PLA2 compared to levels of other soluble markers would indicate the presence of inflammation. The concentration of the PLA2 antigen present in patient samples can be determined using a method that specifically determines the amount of the antigen that is present. Such a method includes an ELISA method in which, for example, antibodies of the invention may be conveniently immobilized on an insoluble matrix, such as a polymer matrix. Alternatively, immunohistochemistry staining assays using anti-PLA2 antibodies may be used to determine levels of PLA2 in a sample. Using a population of samples that provides statistically significant results for each stage of progression or therapy, a range of concentrations of the antigen that may be considered characteristic of each stage of disease can be designated.

In one embodiment, a sample of blood is taken from the subject and the concentration of the PLA2 antigen present in the sample is determined to evaluate the stage of the disease in a subject under study, or to characterize the response of the subject to a course of therapy. The concentration so obtained is used to identify in which range of concentrations the value falls. The range so identified correlates with a stage of disease progression or a stage of therapy identified in the various populations of diagnosed subjects, thereby providing a stage in the subject under study.

Gene amplification and/or expression may be measured in a sample directly, for example, by conventional Southern blotting, Northern blotting to quantitate the transcription of mRNA (Thomas, Proc. Natl. Acad. Sci. USA, 77:5201-5205 (1980)), dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay can be carried out where the duplex is bound to a surface, so that upon the formation of duplex on the surface, the presence of antibody bound to the duplex can be detected.

For example, antibodies, including antibody fragments, can be used to qualitatively or quantitatively detect the expression of PLA2 proteins. As noted above, the antibody preferably is equipped with a detectable, e.g., fluorescent label, and binding can be monitored by light microscopy, flow cytometry, fluorimetry, or other techniques known in the art. These techniques are particularly suitable if the amplified gene encodes a cell surface protein, e.g., a growth factor. Such binding assays are performed as known in the art.

In situ detection of antibody binding to the PLA2 protein can be performed, for example, by immunofluorescence or immunoelectron microscopy. For this purpose, a tissue specimen is removed from the patient, and a labeled antibody is applied to it, preferably by overlaying the antibody on a biological sample. This procedure also allows for determining the distribution of the marker gene product in the tissue examined. It will be apparent for those skilled in the art that a wide variety of histological methods are readily available for in situ detection.

One of the most sensitive and most flexible quantitative methods for quantitating differential gene expression is RT-PCR, which can be used to compare mRNA levels in different sample populations, in normal and tumor tissues, with or without drug treatment, to characterize patterns of gene expression, to discriminate between closely related mRNAs, and to analyze RNA structure.

The first step is the isolation of mRNA from a target sample. The starting material is typically total RNA isolated from a disease tissue and corresponding normal tissues, respectively. Thus, mRNA can be extracted, for example, from frozen or archived paraffin-embedded and fixed (e.g. formalin-fixed) samples of diseased tissue for comparison with normal tissue of the same type. Methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest., 56:A67 (1987), and De Andrés et al., BioTechniques, 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Total RNA from tissue samples can be isolated using RNA Stat-60 (Tel-Test).

As RNA cannot serve as a template for PCR, the first step in differential gene expression analysis by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, CA, USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ endonuclease activity. Thus, TaqMan PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan RT-PCR can be performed using commercially available equipment, such as, for example, ABI PRIZM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRIZM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct). The ΔCt values are used as quantitative measurement of the relative number of starting copies of a particular target sequence in a nucleic acid sample when comparing the expression of RNA in a cell from a diseased tissue with that from a normal cell.

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

Differential gene expression can also be identified, or confirmed using the microarray technique. In this method, nucleotide sequences of interest are plated, or arrayed, on a microchip substrate. The arrayed sequences are then hybridized with specific DNA probes from cells or tissues of interest.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip selectively hybridize to each spot of DNA on the array. After stringent washing to remove non-specifically bound probes, the chip is scanned by confocal laser microscopy. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA, 93(20)L106-49). The methodology of hybridization of nucleic acids and microarray technology is well known in the art.

Characterization of Monoclonal Antibodies

Monoclonal antibodies can be used in a variety of assays as highly specific probes to correlate specific structural determinants with functional properties, making mAbs important tools for validating drug targets. Characterization of protein expression patterns using Ab reagents in western blotting, flow cytometry, and immunohistochemistry is standard practice. The fine specificity of mAbs has allowed the differentiation of closely related molecules, as evidenced by the serotyping of bacterial and viral strains, (Bash, M. C. et al., “Genetic and immunologic characterization of a novel serotype 4, 15 strain of Neisseria meningitidis” FEMS Immunol Med Microbiol 29, 169-176 (2000); Jensen, T. H. et al., “Probing the structure of HIV-1 Rev by protein footprinting of multiple monoclonal antibody-binding sites” FEBS Lett 414, 50-54 (1997)) detection of specific posttranslational variants of proteins (Martegani, et al., “A. Structural variability of CD44v molecules and reliability of immunodetection of CD44 isoforms using mAbs specific for CD44 variant exon products” Am J Pathol 154, 291-300 (1999)), and discrimination of proteins with altered conformations (Drbal et al., “A novel anti-CD18 mAb recognizes an activation-related epitope and induces a high-affinity conformation in leukocyte integrins” Immunobiology 203, 687-698 (2001)). This diversity in epitope binding can further manifest into two different mAbs against the same target mediating very different functional activities, e.g., one mAb may be an antagonist, while another has no activity. Sattentau et al., “Epitopes of the CD4 antigen and HIV infection” Science 234, 1120-1123 (1986); Pierres et al., “Clonal analysis of B- and T-cell responses to Ia antigens. I. Topology of epitope regions on I-Ak and I-Ek molecules analyzed with 35 monoclonal alloantibodies” Immunogenetics 14, 481-495 (1981). Importantly, mAbs are finding increasing utility not only as reagents, but also as therapeutics for human disease. Spigel et al., “HER2 overexpressing metastatic breast cancer” Curr Treat Options Oncol 3, 163-174 (2002); Sandborn, et al., “Biologic therapy of inflammatory bowel disease” Gastroenterology 122, 1592-1608 (2002).

Numerous approaches now exist for generating mAbs. Standard hybridoma technology applied to rodents, typically standard strains of laboratory mice, yields panels of tens to hundreds of mAbs of rodent amino acid sequence. Though useful as reagents, rodent-derived mAbs typically make poor therapeutics for humans because of their likely immunogenicity. Antibody engineering through chimerization or humanization yields Abs with increased human-derived content and a lowered probability of eliciting an immune response in humans, but often at the expense of potency or speed of development. A more expedient route to generating mAbs suitable for human therapeutics is to derive antigen-specific mAbs that are fully human. Importantly, such fully human mAbs may serve both as reagents for target validation and as candidates for therapeutic leads. The two most prevalent technologies for generating fully human Abs are phage display and transgenic mice. Naive human Ab libraries expressed using various display technologies can be sources of antigen-specific Ab fragments, though the fragments typically require subsequent in vitro engineering to meet activity criteria required for therapeutic mAbs. Watkins et al., “Introduction to antibody engineering and phage display” Vox Sang 78, 72-79 (2000). With the engineering of transgenic mice that produce fully human Abs, affinity-matured antigen-specific mAbs suitable as reagents and therapeutics can be recovered directly using hybridoma technology. Mendez et al., “Functional transplant of megabase human immunoglobulin loci recapitulates human antibody response in mice” Nat Genet 15, 146-156 (1997); LaRochelle. et al., “Platelet-derived growth factor D: tumorigenicity in mice and dysregulated expression in human cancer” Cancer Res 62, 2468-2473 (2002); Ishida et al., “Production of Human Monoclonal and Polyclonal Antibodies in TransChromo Animals” Cloning Stem Cells 4, 91-102 (2002); Davis et al., “Transgenic mice as a source of fully human antibodies for the treatment of cancer” Cancer Metastasis Rev 18, 421-425 (1999).

Regardless of the approach taken to generate the initial pool of Abs, there are a finite number of binding specificities. Whether the entire panel of Abs is used in high-throughput functional screening or whether capacity constraints limit the number of Abs that can be screened in functional assays, it may be advantageous to characterize and subsequently reduce a redundancy of binding specificities within the Ab pool. Also, a method for efficient selection of Abs with distinct binding specificities could be used to map functional activities into bins based on binding competition.

The conventional approach to characterizing the binding specificity of an antibody is epitope mapping. Baerga-Ortiz et al., “Epitope mapping of a monoclonal antibody against human thrombin by H/D-exchange mass spectrometry reveals selection of a diverse sequence in a highly conserved protein” Protein Sci 11, 1300-1308 (2002); McCullough et al., “Immune protection against foot-and-mouth disease virus studied using virus-neutralizing and non-neutralizing concentrations of monoclonal antibodies” Immunology 58, 421-428 (1986). Although this technique can identify the site by which each Ab binds to its antigen to high resolution, the process can be time and labor intensive and can have a low throughput. Historically, ELISA-based competition of binding of labeled Ab to an antigen by an excessive amount of unlabeled Ab has been used to determine whether two Abs competed. McCullough et al., supra (1986). Although the ELISA-based competition assay lacks the precision of epitope mapping, it is easy to perform and can be completed in one or two days. However, the assay becomes cumbersome with a large number of samples, and it requires significant amounts of purified Ab.

To this end, a novel method of high-throughput Multiplexed Competitive Antibody Binning (MCAB) has been developed which uses only a small amount of antibody and antigen. The MCAB method allows high-throughput binning of a panel of Abs based on their competition for binding to an antigen and has been successfully used routinely for Ab screening and characterization. The assay is based on similarities of competition binding patterns of Abs on a target molecule and employs Luminex® spectrally encoded beads and detection technology to highly multiplex the assay. The MCAB assay is sensitive enough to be used to screen and characterize Abs from the early stages of hybridoma generation with small amounts of Abs in supernatants in a high-throughput fashion. This assay is also applicable for recombinant Ab fragments generated using display technologies.

This method enables the sorting of a large, complex panel of monoclonal antibodies into different bins based on cross-competition for antigen binding. In some embodiments, the MCAB method is applicable immediately after identification of antigen-positive Abs, providing information useful for advancing Ab candidates into further testing. Alternatively, the MCAB assay can be used for sorting Abs into binding groups after screening for functional activity.

MCAB technology was developed with fully human mAbs derived from XenoMouse® G2 and G4 mice, mouse strains transgenic for human Ab loci that make fully human IgG₂κ and IgG₄κ Abs, respectively. Mendez et al., supra (1997). Immunization of these mice with protein antigens and subsequent generation of hybridomas can routinely result in more than one hundred antigen-specific, high-affinity mAbs. The MCAB method described herein incorporates a multiplexing strategy that uses a bead-based technology to detect antibody (Ab) competition developed by Luminex® (Fulton et al, supra (1997)) and specific capture and detection reagents. Together, the association of each mAb with a unique spectrally encoded bead from 100 commercially available beads enables competition of each mAb against up to 99 other mAbs in a single solution-phase multiplex assay. The method is able to use hybridoma supernatants without purification and can be incorporated early in the antibody generation process.

The MCAB process can be particularly valuable when the monoclonal antibodies themselves are to be used as tools for validation of targets from genomics or proteomics discovery efforts. Quinn-Senger et al., supra (2002); Walke et al., supra (2001). In these cases, there may be no defined functional assays that can be used for advancing candidates from amongst tens or hundreds of antigen-specific Abs after the primary and secondary binding screens. Because the biological function of a given Ab depends on the epitope to which it binds on a target molecule (Blanpain et al., “Multiple active states and oligomerization of CCR5 revealed by functional properties of monoclonal antibodies” Mol Biol Cell 13, 723-737 (2002); Corada et al., “Monoclonal antibodies directed to different regions of vascular endothelial cadherin extracellular domain affect adhesion and clustering of the protein and modulate endothelial permeability” Blood 97, 1679-1684 (2001)), it is useful to group the antibodies into bins based on binding competition, and then to advance a subset of Abs from each bin into functional assays. The definition of non-competing bins also allows for combination of antibodies to be tested for synergistic activities. Kawashima. et al., “The multi-epitope approach for immunotherapy for cancer: identification of several CTL epitopes from various tumor-associated antigens expressed on solid epithelial tumors” Hum Immunol 59, 1-14 (1998).

Selected embodiments of the antibodies and methods are illustrated in the Examples below:

### EXAMPLES

The following examples, including the experiments conducted and results achieved are provided for illustrative purposes only and are not to be construed as limiting upon the embodiments of the invention described herein.

### Example 1

### PLA2 Antigen Preparation

Recombinant PLA2 proteins were prepared using a 26 kDa tag, Glutathionine S-transferase (GST), fused to recombinant PLA2 to facilitate purification from the E. coli expression vector through the use of an affinity matrix which contains glutathionine. Elution of the purified protein was accomplished under mild, nondenaturing conditions well known in the art. Further testing to verify retention of enzymatic activity post-purification was performed and is summarized below. The tag was removed through the incorporation of an endopeptidase cleavage site sequence between the tag and the recombinant PLA2.

### Example 2

### Anti-PLA2 Antibodies

Monoclonal antibodies which bind substantially to PLA2 were developed by immunizing XenoMouse® mice (Abgenix Inc, Fremont, Calif.) with the GST-PLA2 fusion protein to stimulate an immune response. Two strains of mice, an hlgG2 strain (Group 1) and an hlgG4 strain (Group 2), were immunized in the footpad with approximately 0.6 mg/ml, endotoxin concentration free, GST-PLA2, which was purified from an E. coli expression vector. The two groups of ten mice were given a total of 8 boosts over a 30-day period and bled on days 15, 22, and 30. See Table 2. The adjuvants used were: Titermax Gold Adjuvant (Catalog # T-2684, lot # 12K1599, Sigma, 50/50 by volume) for the first boost; Aluminium Phosphate Gel Adjuvant (Catalog # 1452-250, Batch 8919, Superfos Biosector, 5 μl/mouse) and CpG (ODN 1826, 2 mg/ml in D-PBS, 10 μg/mouse) for boosts 2 through 7; and D-PBS for the final boost. Monoclonal antibodies directed against the PLA2 protein fragment were prepared by hybridoma technology from PLA2 protein fragment-immunized XenoMouse® animals in standard fashion.

TABLE 2
Immunization schedule of PLA2 for groups 1 & 2
Target = PLA2; Mode of Immunization = footpad; Antigen = PLA2

| Day | Action | Amount | Adjuvants |
|---|---|---|---|
| 1 | 1^{st} boost | 10 μg/mouse | Titermax Gold |
| 5 | 2^{nd} boost | 10 μg/mouse | Alum + CpG |
| 8 | 3^{rd} boost | 10 μg/mouse | Alum + CpG |
| 12 | 4^{th} boost | 10 μg/mouse | Alum + CpG |
| 15 | Bleed | N/A | N/A |
| 16 | 5^{th} boost | 10 μg/mouse | Alum + CpG |
| 19 | 6^{th} boost | 10 μg/mouse | Alum + CpG |
| 22 | Bleed | N/A | N/A |
| 23 | 7^{th} boost | 10 μg/mouse | Alum + CpG |
| 26 | 8^{th} boost | 10 μg/mouse | D-PBS |
| 30 | Fusion | N/A | N/A |

### Example 3

### ELISA Example

For the determination of anti-PLA2 titer, PLA2 antigen was biotinylated and further coated onto plates for ELISA determination. Briefly, 15-500 μg of PLA2 antigen was diluted in 1 mL of PBS at a pH of 8.6. 10 μL of 10 mg/mL sulfo-NHS-biotin (Biotin stock in DMSO) was added to the 1 mL PLA2 antigen solution and incubated at room temperature with rotation for 1 hour. After incubation, the reaction was quenched with 100 μL of saturated Tris and subjected to centrifugation and a minimum of 4 washes to separate free biotin from the biotinylated PLA2 antigen. The biotinylated PLA2 (1 μg/mL) was coated onto Sigma Streptavidin plates for 1 hour at room temperature. A control Streptavidin plate was left uncoated for use as a control.

The plates were washed five times with distilled water. The hybridoma culture supernatants were titrated in 2% Milk/PBS at 1:2 dilutions in duplicate from a 1:100 initial dilution. The last well was left blank as a control. The streptavidin plates were washed fives times with distilled water. A goat anti-human IgG Fc-specific HRP-conjugated antibody was added at a final concentration of 1 μg/mL for 1 hour at room temperature. Following five washes with distilled water, the Streptavidin plates were developed with the addition of TMB for 30 minutes and the ELISA was stopped by the addition of 1 M phosphoric acid. The specific titer of the hybridoma culture supernatants were determined from the optical density at 450 nm.

### Example 4

### Second Screen for Fully Human Antibodies Against PLA2

Luminex® Assay

To further verify the production of fully human anti-PLA2 antibodies, PLA2 antigen was coated onto the surface of Luminex®t Beads (MiraiBio, Inc., Alameda, Calif.) for multi-analyte bioassay detection.

Briefly, 50 μl/well of a conjugated bead mixture was added to a filter plate and aspirated. Following aspiration, 50 μl/well of samples and controls were added to a filter plate and incubated in the dark on a plate shaker for 20 minutes. After incubation, the filter plate was washed with 100 mL/well wash buffer two times. Finally, 80 μl/well of detection antibody mixture was added and incubated in the dark on a plate shaker for 20 minutes.

The coated beads were probed with a labeled antibody selected from the group consisting of: human gamma, human kappa, human lamda, human IgM, murine gamma, and murine lamda. The bead stock was then gently vortexed and the volume of bead mixture needed calculated using the following formula: (n+6)×50 μl (where n=the number of samples). Next, the beads were diluted in blocking buffer to a concentration of 2500 of each bead per well or 0.5×10⁵/mL. The filter plate was pre-wetted by adding 200 μl wash buffer per well and aspirated.

After incubation, plates were read using a Luminex® machine, which uses microfluidics to align the beads in single file and lasers to illuminate the colors inside and on the surface of each bead. Antibodies that bound to the beads and were found to be fully human were selected for further analysis.

### Example 5

### Structural Analysis of Anti-PLA2 Antibodies

To analyze the structures of antibodies that were fully human and bound to PLA2, genes encoding the heavy and light chain fragments that produced the antibodies were cloned from the corresponding hybridomas. Gene cloning and sequencing was accomplished as follows:

Poly(A)⁺ mRNA was isolated from approximately 2×10⁵ hybridoma cells derived from immunized XenoMouse® mice using a Fast-Track kit (Invitrogen). The generation of random primed cDNA was followed by PCR. Human V_{H} or human V_{κ} family specific variable region primers (Marks et. al., 1991) or a universal human V_{H} primer, MG-30 (CAGGTGCAGCTGGAGCAGTCIGG SEQ ID NO: 32) was used in conjunction with primers specific for the human:

| | |
|---|---|
| Cγ2 constant region (MG-40d; | |
| 5′ | |
| GCT GAG GGA GTA GAG TCC TGA GGA 3′; | SEQ ID NO: 33) |
| Cγ1 constant region (HG1; | |
| 5′ CAC ACC GCG GTC ACA TGG C 3′; | SEQ ID NO: 34) |
| or | |
| Cγ3 constant region (HG3; | |
| 5′ CTA CTC TAG GGC ACC TGT CC 3′; | SEQ ID NO: 35) |

or the human CK constant region (hκP2; as previously described in Green et al., 1994). Sequences of human mAb-derived heavy and kappa chain transcripts from hybridomas were obtained by direct sequencing of PCR products generated from poly(A⁺) RNA using the primers described above. PCR products were also cloned into plasmid pCRII using a TA cloning kit (Invitrogen). In addition, both strands were sequenced using Prism dye-terminator sequencing kits and an ABI 377 sequencing machine. All sequences were analyzed by alignments to the “V BASE sequence directory” (Tomlinson et al., MRC Centre for Protein Engineering, Cambridge, UK) using MacVector and Geneworks software programs.

The sequences for the antibody clones and the CDR regions are summarized in Tables 3-7 below.

TABLE 3

Sequence Data for Anti-PLA2 mAb Heavy Chain

| | | | | | | | | |
|---|---|---|---|---|---|---|---|---|
| | | | # | | | | # | |
| Ab | VH | V Seq | Ns | N | D1 | D1 Sequence | Ns | N |

| | | | | | | | | |
|---|---|---|---|---|---|---|---|---|
| 2.8 | VH3-23 | GCGA | 2 | AA | D3-16 | GGGGGA | 5 | CTGGA |
| | (40-333) | | | | (336-341) | | | |
| 2.29 | VH5-51 | GCGA | 1 | T | D1-26 | TGGGACCTACT | 3 | CCT |
| | (46-339) | | | | (341-351) | (SEQ ID NO: 36) | | |
| 1.12 | VH1-2 | GAGA | 3 | TAG | D5-5 | GGATACAGCTATGGT | 3 | CTT |
| | (49-344) | | | | (348-362) | (SEQ ID NO: 37) | | |
| 1.15 | VH6-1 | CAAG | 4 | GAGA | D6-19 | GTATAGCGGTGGCTGG | 5 | AACTT |
| | (49-352) | | | | (357-372) | (SEQ ID NO: 38) | | |
| 1.2 | VH3-23 | GAAA | 8 | GGGTGTGA | D5-12 | CTACGATT | 2 | TT |
| | (61-356) | | | | (365-372) | | | |
| 2.14 | VH4-31 | GAGA | 3 | GGT | D6-6 | TATAGTAGCTCGTCC | 1 | G |
| | (58-356) | | | | (360-374) | (SEQ ID NO: 39) | | |
| 2.2 | VH3-30 | GAGA | 1 | G | D6-13 | ATAGCAGCAGCTGGT | 5 | TCGTC |
| | (25-320) | | | | (322-336) | (SEQ ID NO: 40) | | |
| 2.26 | VH5-51 | CGAG | 7 | CCCCCCC | D6-19 | GGGTATAGCAGT | 10 | TCCTTTTAAA |
| | (61-355) | | | | (363-374) | (SEQ ID NO: 41) | | (SEQ ID NO: 42) |
| 2.1 | VH3-33 | GTGC | N. | - N.A - | - N.A - | - N.A - | 8 | AAAGGGGT |
| | (61-352) | | A | | | | | |
| 2.13 | VH3-48 | GAGA | 4 | GGGT | D1-7 | CTGGAACTAC | 7 | GAAGGGG |
| | (61-356) | | | | (361-370) | (SEQ ID NO: 43) | | |
| 1.4 | VH1-2 | GAGA | 3 | TAG | D5-5 | GGATACAGCTATGGT | 3 | CTT |
| | (26-320) | | | | (324-338) | (SEQ ID NO: 44) | | |
| 1.11 | VH3-23 | GAAA | 3 | GGG | D2-21 | GGTGACT | 8 | ACGATTTT |
| | (49-344) | | | | (348-354) | | | |
| 1.17 | VH5-51 | GAGA | 5 | GAGAC | D1-26 | GTGGG | 0 | |
| | (49-344) | | | | (350-354) | | | |
| 1.1 | VH5-51 | GCGA | 6 | GGGCGA | D3-16 | GGGGGA | 0 | |
| | (58-351) | | | | (358-363) | | | |
| 1.26 | VH3-33 | ACTG | 6 | GTATAG | D6-19 | CAGTGGCTGGTAC | 4 | CGGG |
| | (49-337) | | | | (344-356) | (SEQ ID NO: 45) | | |
| 2.10 | VH3-48 | GAGA | 4 | GGGT | D1-7 | CTGGAACTAC | 7 | GAAGGGG |
| | (61-356) | | | | (361-370) | (SEQ ID NO: 46) | | |
| 2.28 | VH4-39 | TGCG | N. | - N.A - | - N.A - | - N.A - | 2 | CC |
| | (49-344) | | A | | | | | |
| 2.27 | VH3-33 | GCGA | N. | - N.A - | - N.A - | - N.A - | 6 | AGGGGT |
| | (61-354) | | A | | | | | |
| 2.21 | VH3-33 | CGAG | 4 | GGGG | D1-1 | AACTGGAAC | 7 | CCCCGGG |
| | (46-340) | | | | (345-353) | | | |
| 1.9 | VH3-15 | TACC | 3 | CGG | D3-16 | TATGATTACGTTTGGGGGAG | 5 | CCATG |
| | (61-359) | | | | (363-382) | (SEQ ID NO: 47) | | |
| 1.28 | VH1-2 | GAGA | 3 | TAG | D5-5 | GGATACAGCTATGGT | 3 | CTT |
| | (49-344) | | | | (348-362) | (SEQ ID NO: 48) | | |
| 1.22 | VH3-30 | CGAG | 4 | GGGT | D4-23 | CTACGGTG | 0 | |
| | (46-340) | | | | (345-352) | | | |
| 1.19 | VH3-33 | GCGA | N. | - N.A - | - N.A - | - N.A - | 10 | AGGGACTGGA |
| | (46-339) | | A | | | | | (SEQ ID NO: 49) |
| 1.16 | VH1-2 | GAGA | 3 | TAG | D5-5 | GGATACAGCTATGGT | 3 | CTT |
| | (49-344) | | | | (348-362) | (SEQ ID NO: 50) | | |
| 1.13 | VH3-33 | GCGA | 6 | CAGGGG | D2-2 | ATAGCAGCT | 7 | CGTAGAA |
| | (46-339) | | | | (346-354) | | | |
| 1.10 | VH1-8 | GTGC | 8 | AAGAAGGG | D7-27 | AACTGGGG | 3 | GTC |
| | (49-340) | | | | (349-356) | | | |
| 1.6 | VH5-51 | GAGA | 3 | TAC | D1-26 | GGGAGC | 2 | CC |
| | (46-341) | | | | (345-350) | | | |
| 2.6 | VH3-33 | GCGA | 5 | AGGGG | D5-12 | GCCACTAT | 0 | |
| | (46-339) | | | | (345-352) | | | |

| | | J | Constant | | | |
|---|---|---|---|---|---|---|
| Ab | JH | Sequence | Region | CDR1 AA Seq | CDR2 AA Seq | CDR3 AA Seq |
| 2.8 | JH4b | ACTACG | G4 | GFTFSSYAMN | FISGSGGSTYYADSVKG | KGDWNYEDY |
| | (347-394) | | (395-503) | (SEQ ID NO: 51) | (SEQ ID NO: 52) | (SEQ ID NO: 53) |
| 2.29 | JH4b | TTTGAC | G4 | GYSFTSYWIG | IIYPGDSDTRYSPSFQG | LGPTPFDY |
| | (355-397) | | (398-513) | (SEQ ID NO: 54) | (SEQ ID NO: 55) | (SEQ ID NO: 56) |
| 1.12 | JH6b | TTACTA | G2 | GYTFTDYYIH | WIHPNSGGTNYAQKFQG | DRDTAMVFYYYYYAMDV |
| | (366-427) | | (428-496) | (SEQ ID NO: 57) | (SEQ ID NO: 58) | (SEQ ID NO: 59) |
| 1.15 | JH6b | CTACTA | G2 | GDSVSSNSAAWN | RTYYRSKWYNDYAVSVKS | GEYSGGWNFYYYGMDV |
| | (378-433) | | (434-477) | (SEQ ID NO: 60) | (SEQ ID NO: 61) | (SEQ ID NO: 62) |
| 1.2 | JH2 | CTACTG | G2 | GFTFSSYAMS | AISGSGGSTYYADSVKG | EGVTTIFYWYFDL |
| | (375-427) | | (428-541) | (SEQ ID NO: 63) | (SEQ ID NO: 64) | (SEQ ID NO: 65) |
| 2.14 | JH5b | TGGTTC | G4 | GGSISSGGYYWS | YIYYSGSTYYNPSLKS | EVIVARPWFDP |
| | (376-421) | | (422-454) | (SEQ ID NO: 66) | (SEQ ID NO: 67) | (SEQ ID NO: 68) |
| 2.2 | JH6b | CGGTAT | G4 | GFTFSIYGMH | IISYGGSNKYYADSVKG | EIAAAGSSGMDV |
| | (342-388) | | (389-499) | (SEQ ID NO: 69) | (SEQ ID NO: 70) | (SEQ ID NO: 71) |
| 2.26 | JH4b | GACTAC | G4 | GYSFTSYWIG | IIYPGDSDTRYSPSFQG | PPPGIAVPFKDY |
| | (385-424) | | (425-524) | (SEQ ID NO: 72) | (SEQ ID NO: 73) | (SEQ ID NO: 74) |
| 2.1 | JH4b | TTTGAC | G4 | GFTFSSYGMH | IIWYDGSYRFYADSVKG | RGFDY |
| | (361-403) | | (404-419) | (SEQ ID NO: 75) | (SEQ ID NO: 76) | (SEQ ID NO: 77) |
| 2.13 | JH6b | TTACTA | G4 | GFTFSSYSMN | YISSGSSTIYYADSVKG | EGLELRRGYYYYYGMDV |
| | (378-439) | | (440-472) | (SEQ ID NO: 78) | (SEQ ID NO: 79) | (SEQ ID NO: 80) |
| 1.4 | JH6b | TTACTA | G2 | GYTFTGYYMH | WINPNSGGTNYAQKFQG | DRDTAMVFYYYYYALDV |
| | (342-403) | | (404-500) | (SEQ ID NO: 81) | (SEQ ID NO: 82) | (SEQ ID NO: 83) |
| 1.11 | JH2 | CTACTG | G2 | GFTFSSYAMS | AISGSGGSTYYADSVKG | EGVTTIFYWYFDL |
| | (363-415) | | (416-515) | (SEQ ID NO: 84) | (SEQ ID NO: 85) | (SEQ ID NO: 86) |
| 1.17 | JH4b | TTTGAC | G2 | GYSFTSYWIG | IIYPGDSDTRYSPSFQG | QRRGFDY |
| | (355-397) | | (398-531) | (SEQ ID NO: 87) | (SEQ ID NO: 88) | (SEQ ID NO: 89) |
| 1.1 | JH4b | TTTGAC | G2 | GYSFTSYWIA | IIYPGDSDTRYSPSFQG | GRGGFDY |
| | (364-406) | | (407-538) | (SEQ ID NO: 90) | (SEQ ID NO: 91) | (SEQ ID NO: 92) |
| 1.26 | JH3b | GCTTTT | G2 | GFTFSTYGMH | VIWYDGSNKYYADSVKG | AVAGTGAFDI |
| | (361-406) | | (407-507) | (SEQ ID NO: 93) | (SEQ ID NO: 94) | (SEQ ID NO: 95) |
| 2.10 | JH6b | TTACTA | G1 | GFTFSSYSMN | YISSGSSTIYYADSVKG | EGLELRRGYYYYYGMDV |
| | (378-439) | | (440-455) | (SEQ ID NO: 96) | (SEQ ID NO: 97) | (SEQ ID NO: 98) |
| 2.28 | JH4b | TTGACT | G4 | GGSISRSSYYWG | SIYYSGSTYYNPSLKS | LDY |
| | (347-388) | | (389-515) | (SEQ ID NO: 99) | (SEQ ID NO: 100) | |
| 2.27 | JH4b | TTTGAC | G4 | GFTFSNYGIH | VIWYDGSYKFYADSVKG | RGFDS |
| | (361-403) | | (404-526) | (SEQ ID NO: 101) | (SEQ ID NO: 102) | (SEQ ID NO: 103) |
| 2.21 | JH3b | GCTTTT | G4 | GFTFSSYGMH | AIWYDGSNKYYADSVKG | GGTGTPGAFDI |
| | (361-406) | | (407-517) | (SEQ ID NO: 104) | (SEQ ID NO: 105) | (SEQ ID NO: 106) |
| 1.9 | JH4b | TTTGAC | G2 | GFIFSNAWMS | RIKSKTDGGTTDYAAPVKG | GMITFGGAMFDF |
| | (388-430) | | (431-521) | (SEQ ID NO: 107) | (SEQ ID NO: 108) | (SEQ ID NO: 109) |
| 1.28 | JH6b | TTACTA | G2 | GYTFNDYYMH | WIHPNSGGTNYAQKFQG | DRDTAMVFYYYYYAMDV |
| | (366-427) | | (428-511) | (SEQ ID NO: 110) | (SEQ ID NO: 111) | (SEQ ID NO: 112) |
| 1.22 | JH4b | ACTTTG | G2 | GFTFRSYGMH | VISYDGSNKYYADSVKG | GVYGDFDY |
| | (353-397) | | (398-522) | (SEQ ID NO: 113) | (SEQ ID NO: 114) | (SEQ ID NO: 115) |
| 1.19 | JH6b | ACTACG | G2 | GFTFSNYGMH | VIWYDGSNKYYADSVKG | RDWNYGMDV |
| | (350-400) | | (401-525) | (SEQ ID NO: 116) | (SEQ ID NO: 117) | (SEQ ID NO: 118) |
| 1.16 | JH6b | TTACTA | G2 | GYTFTDYYMH | WISPNSGGTNYAQKFQG | DRDTAMVFYYYYYAMDV |
| | (366-427) | | (428-484) | (SEQ ID NO: 119) | (SEQ ID NO: 120) | (SEQ ID NO: 121) |
| 1.13 | JH6b | ACTACT | G2 | GFTFSSYGMH | VIWYDGSNKYYADSVKG | QGIAARRNYYYSGMDV |
| | (362-421) | | (422-513) | (SEQ ID NO: 122) | (SEQ ID NO: 123) | (SEQ ID NO: 124) |
| 1.10 | JH4b | CTTTGA | G2 | GYTFTSYDIN | WMDPNSGHTGYAQKFQG | EGNWGSFDY |
| | (360-403) | | (404-514) | (SEQ ID NO: 125) | (SEQ ID NO: 126) | (SEQ ID NO: 127) |
| 1.6 | JH4b | TTGACT | G2 | GYSFTNYWIG | FIYPGDSDTRYSPSFEG | HTGALDY |
| | (353-394) | | (395-520) | (SEQ ID NO: 128) | (SEQ ID NO: 129) | (SEQ ID NO: 130) |
| 2.6 | JH3b | ATGCTT | G4 | GITFSSYGMH | VIWYDGSNKYYVDSVKG | RGPLYAFDI |
| | (353-400) | | (401-517) | (SEQ ID NO: 131) | (SEQ ID NO: 132) | (SEQ ID NO: 133) |

TABLE 4

Sequence Data by Class for Anti-PLA2 mAb Heavy Chain

| | | | | | | | |
|---|---|---|---|---|---|---|---|
| SEQ | | | | | | | |
| ID | | | | | | | |
| NO | Ab | V | D | J | FR1 | CDR1 | FR2 |

| | | | | | | | |
|---|---|---|---|---|---|---|---|
| 134 | | | Germlines | | EVQLVQSGAEVKKPGESLKISCKGS | GYSFTSYWIG | WVRQMPGKGLEWMG |
| 15 | 2.7 | VH5-51 | D3-16 | JH3b | GVQLVQSGAEVKKPGESLKISCKGS | GYSFTNYWIG | WVRQMPGKGLEWMG |
| 135 | | | Germlines | | EVQLVQSGAEVKKPGESLKISCKGS | GYSFTSYWIG | WVRQMPGKGLEWMG |
| 17 | 2.9 | VH5-51 | D6-6 | JH3b | EVQLVQSGAGVKKPGESLKISCKGS | GYSFTSYWIN | WVRQMPGKGLEWMG |
| 27 | 2.25 | ″ | ″ | ″ | EVQLVQSGAEVKKPGESLKISGKGS | GYSFISYWIA | WVRQMPGKGLEWMG |
| 30 | 2.4 | ″ | ″ | ″ | EVQLVQSGAEVKKPGESLKISCKGS | GYSFTSYWIG | WLRQMPGKGLEWMG |
| 136 | | | Germlines | | QVQLVESGGGVVQPGRSLRLSCAAS | GFTFSSYGMH | WVRQAPGKGLEWVA |
| 23 | 2.19 | VH3-33 | D1-I | JH3b | QVQLVESGGGVVQPGRSLRLSCAAS | GFTFSSYGMH | WVRQAPGKGLEWVA |
| 137 | | | Germlines | | QVQLVESGGGVVQPGRSLRLSCAAS | GFTFSSYGMH | WVRQAPGKGLEWVA |
| 21 | 2.15 | VH3-33 | D1-7 | JH3b | QVQLVESGGGVVQPGRSLRLSCAAS | GFTFSSYGMH | WVRQAPGKGLEWVA |
| 138 | | | Germlines | | EVQLVQSGAEVKKPGESLKISCKGS | GYSFTSYWIG | WVRQMPGKGLEWMG |
| 9 | 1.18 | VH5-51 | D6-19 | JH4b | EVQLVQSGAEVKKPGESLKISCKGS | GYSFTNYWIN | WVRQMPGKGLEWMG |
| 139 | | | Germlines | | EVQLVQSGAEVKKPGESLKISCKGS | GYSFTSYWIG | WVRQMPGKGLEWMG |
| 5 | 1.7 | VH5-51 | D2-2 | JH6b | EVQLVQSGAEVKKPGESLKISCKGS | GYSFISYWIG | WVRQMPGKGLEWMG |
| 13 | 1.27 | ″ | ″ | ″ | EVQLVQSGAEVKKPGESLKISCKGS | GYSFTSYWIG | WVRQMPGKGLEWMG |
| 140 | | | Germlines | | EVQLVQSGAEVKKPGESLKISCKGS | GYSFTSYWIG | WVRQMPGKGLEWMG |
| 3 | 1.5 | VH5-51 | D2-8 | JH6b | EVQLVQSGAEVKKPGESLKISCKGS | GYSFISYWIG | WVRQMPGKGLEWMG |
| 141 | | | Germlines | | EVQLVQSGAEVKKPGESLKISCKGS | GYSFTSYWIG | WVRQMPGKGLEWMG |
| 19 | 2.12 | VH5-51 | D1-7 | JH3b | EVQLVQSGAEVKKPGESLKISCKGS | GYNFITYWIA | WVRQMPGKGLEWMG |
| 142 | | | Germlines | | QVQLVESGGGVVQPGRSLRLSCAAS | GFTFSSYGMH | WVRQAPGKGLEWVA |
| 25 | 2.23 | VH3-33 | D3-3 | JH4b | QVQLEESGGGVVQPGRSLRLSCAAS | GFTFSSYGMH | WVRQGPGKGLEWVA |
| 143 | | | Germlines | | EVQLVQSGAEVKKPGESLKISCKGS | GYSFTSYWIG | WVRQMPGKGLEWMG |
| 29 | 1.3 | VH5-51 | | JH6b | EVQLVQSGAEVKKPGESLKISCKGS | GYSFTIYWIG | WVRQMPGKGLEWMG |
| 144 | | | Germlines | | EVQLVQSGAEVKKPGESLKISCKGS | GYSFTSYWIG | WVRQMPGKGLEWMG |
| 11 | 1.21 | VH5-51 | | JH3b | EVQLVQSGAEVKKPGESLKISCKGS | GYRFTSYWIG | WVRQMPGKGLEWMG |
| 145 | | | Germlines | | EVQLVQSGAEVKKPGESLKISCKGS | GYSFTSYWIG | WVRQMPGKGLEWMG |
| 7 | 1.14 | VH5-51 | D1-26 | JH4b | EVQLVQSGAEVKKPGESLKISGKGS | GYSITSYWIG | WVRQMPGKGLEWMG |
| 31 | 2.16 | ″ | ″ | ″ | EVQLVQSGAEVKKPGESLKISCKGS | GYSFTSYWIN | WVRQMPGKGLEWMG |

| | | | | |
|---|---|---|---|---|
| Ab | CDR2 | FR3 | CDR3 | J |

| | | | | |
|---|---|---|---|---|
| | IIYPGDSDTRYSPSFQG | QVTISADKSISTAYLQWSSLKASDTAMYYCAR | GG##AFDI | WGQGTMVTVSSA |
| 2.7 | IIYPGDSDTRYSPSFQG | QVTISADKSISTAYLQWSSLKASDTAIYYCAR | GGVGAFDI | WGQGTMVTVSSA |
| | IIYPGDSDTRYSPSFQG | QVTISADKSISTAYLQWSSLKASDTAMYYCAR | SSS#AFDI | WGQGTMVTVSSA |
| 2.9 | IIYPGDSDTRYSPSFQG | QVTISADKS1STAYLQWSSLKASDTAMYYCAR | STSSAFDI | WGQGTMVTVSSA |
| 2.25 | IIYPGDSDARYSPSFQG | QVTISADKSISTAYLQWSSLKASDTAMYYCAR | TTSDAFDI | WGQGTMVTVSSA |
| 2.4 | IIYPGDSDTRYSPSFQG | QVTISADKSISTAYLQWSSLKASDTAMYYCAR | STS#AFDI | WGQGTMVTVSSA |
| | VIWYDGSNKYYADSVKG | RFTISRDNSKNTLYLQMNSLRAEDTAVYYCAR | ##TG###AFDI | WGQGTMVTVSSA |
| 2.19 | AIWYDGSNKWYADSVKG | RFTISRDNSKNTLYLQMNSLRAEDTAVYYCAR | GGTGTPGAFDI | WGQGTMVTVSSA |
| | VIWYDGSNKYYADSVKG | RFTISRDNSKNTLYLQMNSLRAEDTAVYYCAR | ##WNYAFDI | WGQGTMVTVSSA |
| 2.15 | VIWYDGSNKYYADSVKG | RFTISRDNSKNTLYLQMNSLRAEDTAVYYCAR | RDWNYAFDI | WGQGTMVTVSSA |
| | IIYPGDSDTRYSPSFQG | QVTISADKSISTAYLQWSSLKASDTAMYYCAR | ##L#FDY | WGQGTLVTVSSA |
| 1.18 | IIYPGDSDTRYSPSFQG | QVTISADKS1STAYLQWSSLKASDTAMYYCAR | HRLGFDY | WGQGTLVTVSSA |
| | IIYPGDSDTRYSPSFQG | QVTISADKSISTAYLQWSSLKASDTAMYYCAR | SW#YGMDV | WGQGTTVTVSSA |
| 1.7 | IIYPGDSDTRYSPSFQG | QVTISADKSISTAYLQWSSLKASDTAMYYCAR | SWTYALDV | WGQGTAVTVSSA |
| 1.27 | IIYPGDSDTRYSPSFQG | QVTISADKSISTAYLQWSSLKASDTAMYYCAR | SWTYGMDV | WGQGTTVTVSSA |
| | IIYPGDSDTRYSPSFQG | QVTISADKSISTAYLQWSSLKASDTAMYYCAR | #WCYGMDV | WGQGTTVTVSSA |
| 1.5 | IIYPGDSDTRYSPSFQG | QVTISADKSISTAYLQWSSLKASDTAMYYCAR | HWSYGMDV | WGQGTTVTVSSA |
| | IIYPGDSDTRYSPSFQG | QVTISADKSISTAYLQWSSLKASDTAMYYC## | TGT#AFDI | WGQGTMVTVSSA |
| 2.12 | IIYPGDSDTRYSPSFQG | QVTISADKSISTAYLQWSSLKASDTAMYYCAL | TGTRAFEI | WGQGTMVTVSSA |
| | VIWYDGSNKYYADSVKG | RFTISRDNSKNTLYLQMNSLRAEDTAVYYCAR | ##TIFGVVIDY | WGQGTLVTVSSA |
| 2.23 | VIWYDGSNKKYADSVKG | RFTISRDNSKNTLYLQMNSLRAEDTAVYYCAR | DGPIFGVVMGY | WGQGTLVTVSSA |
| | IIYPGDSDTRYSPSFQG | QVTISADKSISTAYLQWSSLKASDTAMYYCAR | ##YYGMDV | WGQGTTVTVSSA |
| 1.3 | IIYPGDSDTRYSPSFQG | QVTISADQSISTAYLQWSSLKASDTAMYYCAR | HDSYGMDV | WGQGTTVTVSSA |
| | IIYPGDSDTRYSPSFQG | QVTISADKSISTAYLQWSSLKASDTAMYYCAR | ###AFDI | WGQGTMVTVSSA |
| 1.21 | IIYPGDSDTRYSPSFQG | QVTISADKSISTAYLQWSSLKASDTAMYYCAR | HREAFDI | WGQGTMVTVSSA |
| | IIYPGDSDTRYSPSFQG | QVTISADKSISTAYLQWSSLKASDTAMYYCAR | HSGSYFDY | WGQGTLVTVSSA |
| 1.14 | IIYPGDSDTRYSPSFQG | QVTISADKSISTAYLQWSSLKASDTAMYYCAR | HSGSSFDY | WGQGTLVTVSSA |
| 2.16 | IIYPGDSDTRYSPSFQG | QVTISADKSISTAYLQWSSLKASDTAMYYCAR | HVRSPFDY | WGQGTLVTVSSA |

TABLE 5

Sequence Data for Anti-PLA2 mAb Light Chain

| | | | | | | | |
|---|---|---|---|---|---|---|---|
| Ab | VL | V Seq | #Ns | N | JL | J Seq | Constant Region |

| | | | | | | | |
|---|---|---|---|---|---|---|---|
| 2.26 | A27 (49-334) | GCTGAC | 1 | A | JK5 (336-373) | GATCAC | IGKC (374-485) |
| 2.1 | A30(34-319) | AGCCTC | 0 | | JK4 (320-355) | TCACTT | IGKC (356-491) |
| 1.15 | A30 (34-317) | TTACCC | 2 | GC | JK4 (320-355) | TCACTT | IGKC (356-491) |
| 1.2 | B3 (28-331) | CTCGTC | 0 | | JK1 (332-367) | GGACGT | IGKC (368-503) |
| 2.6 | A27 (52-341) | ACCTCC | 6 | GTGCAG | JK2 (348-379) | TTTTGG | IGKC (380-515) |
| 1.26 | A27 (49-335) | GTCACC | 0 | | JK5 (336-373) | GATCAC | IGKC (374-524) |
| 1.11 | B3 (46-349) | CTCCTC | 0 | | JK1 (350-385) | GGACGT | IGKC (386-521) |
| 1.17 | A3 (49-349) | CTCCTC | 0 | | JK4 (350-385) | TCACTT | IGKC (386-498) |
| 1.20 | L2 (49-335) | GCCTCC | 6 | GTGCAG | JK2 (342-373) | TTTTGG | IGKC (374-511) |
| 1.23 | L2 (49-333) | TGGCCT | 0 | | JK5 (334-370) | ATCACC | IGKC (371-489) |
| 2.22 | A3 (49-347) | AACTCC | 0 | | JK3 (348-385) | ATTCAC | IGKC (386-521) |
| 2.28 | O18 (49-332) | TCTCCC | 0 | | JK5 (333-370) | GATCAC | IGKC (371-408) |
| 1.1 | B3 (46-343) | ATAGTA | 4 | GTCC | JK1 (348-385) | GTGGAC | IGKC (386-521) |
| 1.6 | B3 (49-352) | TTCCTC | 0 | | JK4 (353-388) | TCACTT | IGKC (389-524) |
| 1.9 | B3 (49-352) | CTCCTC | 0 | | JK1 (353-388) | GGACGT | IGKC (389-526) |
| 1.22 | A30 (37-323) | CCCTCC | 1 | T | JK4 (325-358) | ACTTTC | IGKC (359-505) |
| 1.19 | A27 (52-341) | ACCTCC | 6 | GTGCAG | JK2 (348-379) | TTTTGG | IGKC (380-513) |
| 1.16 | A2 (37-337) | TTCCTC | 0 | | JK4 (338-373) | TCACTT | IGKC (374-510) |
| 1.10 | B3 (52-355) | TTCCTC | 0 | | JK1 (356-391) | GGACGT | IGKC (392-527) |
| 1.28 | A2 (49-349) | TTCCTC | 0 | | JK4 (350-385) | TCACTT | IGKC (386-520) |
| 2.18 | A3 (34-330) | CAAACT | 0 | | JK5 (331-367) | ATCACC | IGKC (368-503) |

| | | | | | | |
|---|---|---|---|---|---|---|
| Ab | CDR1 | CDR2 | CDR3 | CDR1 AA Seq | CDR2 AA Seq | CDR3 AA Seq |

| | | | | | | |
|---|---|---|---|---|---|---|
| 2.26 | 118-153 | 199-219 | 316-342 | RASQSVSSRYLA | GASSRAT | QQYGSSQIT |
| | | | | (SEQ ID NO: 146) | (SEQ ID NO: 147) | (SEQ ID NO: 148) |
| 2.1 | 103-135 | 181-201 | 298-324 | RASQGISNDLA | AASSLQS | LQHNSYPLT |
| | | | | (SEQ ID NO: 149) | (SEQ ID NO: 150) | (SEQ ID NO: 151) |
| 1.15 | 103-135 | 181-201 | 298-324 | RASQGIRNDLG | AASSLQS | LQHNIYPLT |
| | | | | (SEQ ID NO: 152) | (SEQ ID NO: 153) | (SEQ ID NO: 154) |
| 1.2 | 97-147 | 193-213 | 310-336 | KSSQSVLYSSNNKNYLT | WASTRES | QQYYSTPRT |
| | | | | (SEQ ID NO: 155) | (SEQ ID NO: 156) | (SEQ ID NO: 157) |
| 2.6 | 121-156 | 202-222 | 319-348 | RASQSVSSRYLA | GASSRAA | QQCDYSPPCS |
| | | | | (SEQ ID NO: 158) | (SEQ ID NO: 159) | (SEQ ID NO: 160) |
| 1.26 | 118-153 | 1991-219 | 316-342 | RASQSVRKSYLA | GASSRAT | QQYDYSPIT |
| | | | | (SEQ ID NO: 161) | (SEQ ID NO: 162) | (SEQ ID NO: 163) |
| 1.11 | 115-165 | 211-231 | 328-354 | KSSQSVLYSSNNKNYLA | WASTRES | QQYYSTPRT |
| | | | | (SEQ ID NO: 164) | (SEQ ID NO: 165) | (SEQ ID NO: 166) |
| 1.17 | 118-165 | 211-231 | 328-354 | RSSQSLLQSNGYKYLE | LGSNRAS | MQALQTPLT |
| | | | | (SEQ ID NO: 167) | (SEQ ID NO: 168) | (SEQ ID NO: 169) |
| 1.20 | 118-150 | 196-216 | 313-342 | RASQSVSSNLA | GASTRAT | QQYNNWPPCS |
| | | | | (SEQ ID NO: 170) | (SEQ ID NO: 171) | (SEQ ID NO: 172) |
| 1.23 | 118-150 | 196-216 | 313-339 | RASQSVSRILA | GASTRAT | QQYHNWPIT |
| | | | | (SEQ ID NO: 173) | (SEQ ID NO: 174) | (SEQ ID NO: 175) |
| 2.22 | 118-165 | 211-231 | 328-354 | RSSQSLLHSNGYNYLD | LGSNRAS | MQALQTPFT |
| | | | | (SEQ ID NO: 176) | (SEQ ID NO: 177) | (SEQ ID NO: 178) |
| 2.28 | 118-150 | 196-216 | 313-339 | QASQDISNYLN | DASNLET | QQYDNLPIT |
| | | | | (SEQ ID NO: 179) | (SEQ ID NO: 180) | (SEQ ID NO: 181) |
| 1.1 | 115-165 | 211-231 | 328-354 | KSSQSVLYSSNNKYFLA | WASTRES | QQYYSSPWT |
| | | | | (SEQ ID NO: 182) | (SEQ ID NO: 183) | (SEQ ID NO: 184) |
| 1.6 | 118-168 | 214-234 | 331-357 | KSSQSVLYRSNNKNFLA | WASTRES | QQHYSIPLT |
| | | | | (SEQ ID NO: 185) | (SEQ ID NO: 186) | (SEQ ID NO: 187) |
| 1.9 | 118-168 | 214-234 | 331-357 | KSSQSVLYSSNNKNYLA | WASTRDS | QQYYSTPRT |
| | | | | (SEQ ID NO: 188) | (SEQ ID NO: 189) | (SEQ ID NO: 190) |
| 1.22 | 106-138 | 184-204 | 301-327 | RASQGIRNDLA | AASSLQS | LQHNSYPPT |
| | | | | (SEQ ID NO: 191) | (SEQ ID NO: 192) | (SEQ ID NO: 193) |
| 1.19 | 121-156 | 202-222 | 319-348 | RASQSVSSSYLA | GASSRAT | QHYGSLPPCS |
| | | | | (SEQ ID NO: 194) | (SEQ ID NO: 195) | (SEQ ID NO: 196) |
| 1.16 | 106-153 | 199-219 | 316-342 | KSSQSLLYSDGKTYLY | EVSNRFS | MQSIQLPLT |
| | | | | (SEQ ID NO: 197) | (SEQ ID NO: 198) | (SEQ ID NO: 199) |
| 1.10 | 121-171 | 217-237 | 334-360 | KSSQSVLFRSNNRNYLA | WASTRES | QQYYSIPRT |
| | | | | (SEQ ID NO: 200) | (SEQ ID NO: 201) | (SEQ ID NO: 202) |
| 1.28 | 118-165 | 211-231 | 328-354 | KSSQSLLHSDGKTYLY | EVSNRFS | MQSIQLPLT |
| | | | | (SEQ ID NO: 203) | (SEQ ID NO: 204) | (SEQ ID NO: 205) |
| 2.18 | 103-150 | 196-216 | 313-336 | RSSQSLLHSNGYNYLD | LGSNRAS | MQALQTIT |
| | | | | (SEQ ID NO: 206) | (SEQ ID NO: 207) | (SEQ ID NO: 208) |

TABLE 6

Sequence Data by Class for Anti-PLA2 mAb Light Chain

| | | | | | | |
|---|---|---|---|---|---|---|
| SEQ | | | | | | |
| ID | | | | | | |
| NO. | Ab | V | J | FR1 | CRD1 | FR2 |

| | | | | | | |
|---|---|---|---|---|---|---|
| 209 | | | Germlines | DIQMTQSPSSLSASVGDRVTITC | RASQSISSYLN | WYQQKPGKAPKLLIY |
| 26 | 2.23 | O12 | JK5 | DIQMTQSPSSLSASVGDRVTITC | RTSQSISNYLN | WFQQKPGKAPILLIY |
| 22 | 2.15 | ″ | ″ | DIQMTQSPSSLSASVGDRVTITC | RASQSISNYLN | WYQQKPGKAPKFLIY |
| 210 | | | Germlines | EIVLTQSPGTLSLSPGERATLSC | RASQSVSSSYLA | WYQQKPGQAPRLLIY |
| 6 | 1.7 | A27 | JK4 | EIVLTQSPGTLSLSPGERATLSC | RPSQSVRSNYLT | WYQQKPGQAPRLLIY |
| 14 | 1.27 | ″ | ″ | EIVLTQSPGTLSLSPGERATLSC | RASQSVRSNYLT | WYQQKPGQAPRLLIY |
| 4 | 1.5 | ″ | ″ | EIVLTQSPGTLSLSPGERATLSC | RASQSVRSGYLA | WYQQRPGQAPRFLIY |
| 211 | | | Germlines | DIQMTQSPSSLSASVGDRVTITC | RASQGIRNDLG | WYQQKPGKAPKRLIY |
| 10 | 1.18 | A30 | JK1 | DIQMTQSPSSLSASVGDRVTITC | RASQGIRNDLD | WCQQKPGKAPKRLIY |
| 212 | | | Germlines | DIVMTQSPLSLPVTPGEPASISC | RSSQSLLHSNGYNYLD | WYLQKPGQSPQLLIY |
| 12 | 1.21 | A3 | JK1 | DIVMTQSPLSLPVTPGEPASISC | RSSQSLLHSNGYNFLD | WYLQKPGQSPQLLIY |
| 213 | | | Germlines | EIVLTQSPGTLSLSPGERATLSC | RASQSVSSSYLA | WYQQKPGQAPRLLIY |
| 16 | 2.7 | A27 | JK3 | EIVLTQSPGTLSLSPGERATLSC | RASQIIRRSSLA | WYQEKPGQAPRLLIY |
| 214 | | | Germlines | DIQMTQSPSSLSASVGDRVTITC | RASQSISSYLN | WYQQKPGKAPKLLIY |
| 28 | 2.25 | O12 | JK1 | DIQMTQSPSSLSASVGDRVTITC | RASQSISSYLN | WYQQKPGKAPKLLIY |
| 18 | 2.9 | ″ | ″ | DIQMTQSPSSLSASVGDRVTITC | RASQSISRYLN | WYQQKPGKAPKLLIY |
| 215 | | | Germlines | DIVMTQSPLSLPVTPGEPASISC | RSSQSLLHSNGYNYLD | WYLQKPGQSPQLLIY |
| 24 | 2.19 | A3 | JK5 | DIVMTQSPLSLPVTPGEPASISC | RSSQSLLHSNGYNYLD | WYLQKPGQSPQLLIY |
| 8 | 1.14 | ″ | ″ | DIVMTQSPLSLPVTPGEPASISC | RSSQSLLHSNGYNYLD | WYLQKPGQSPQLLIY |
| 216 | | | Germlines | DIQMTQSPSSLSASVGDRVTITC | RASQSISSYLN | WYQQKPGK####PKL |
| 20 | 2.12 | O12 | JK3 | DIQMTQSPSSLSASVGDRVTITC | RASQSIGSYLN | WYQQKPGKPGKGPKL |

| | | | | |
|---|---|---|---|---|
| Ab | CDR2 | FR3 | CDR3 | J |

| | | | | |
|---|---|---|---|---|
| | AASSLQS | GVPSRFSGSGSGTDFTLTISSLQPEDFATYYC | QQSYSTPIT | FGQGTRLEIKR |
| 2.23 | AASSLQS | GVPSRFSGSGSGTDFTLTISSLQPEDFATYYC | HQSYSIPIT | FGQGTRLEIKR |
| 2.15 | AASSLQS | GAPSRFSGSGSGTDFTLTISSLQPEDFATYYC | QQSYSTPIT | FGQGTRLEIKR |
| | GASSRAT | GIPDRFSGSGSGTDFTLTISRLEPEDFAVYYC | QQYGSSPLT | FGGGTKVEIKR |
| 1.7 | GASTRAT | GIPDRFSGSGSGTDFTLTVSRLEPEDFAVYYC | QQYGSSPLT | FGGGTKVEIKR |
| 1.27 | GASTRAT | GIPDRFSGSGSGTDFTLTISRLEPEDFAVYYC | QQYGSSPLT | FGGGTKVEIKR |
| 1.5 | GASSRAT | GIPDRFSGSGSGTDFTLTISRLEPEDFAVYYC | QQYGSSPLT | FGGGTKVEIKR |
| | AASSLQS | GVPSRFSGSGSGTEFTLTISSLQPEDFATYYC | LQHNSYPPT | FGQGTKVEIKR |
| 1.18 | AASSLQS | GVPSRFSGSGSGTEFTLTISSLQPEDFATYYC | LQHNNYPPT | FGQGTKVEIKR |
| | LGSNRAS | GVPDRFSGSGSGTDFTLKISRVEAEDVGVYYC | MQALQTPPT | FGQGTKVEIKR |
| 1.21 | LGSNRAS | GVPDRFSGSGSGTDFTLKISRVEAEDVGVYYC | MQALQTPPT | FGPGTKVEIKR |
| | GASSRAT | GIPDRFSGSGSGTDFTLTISRLEPEDFAVYYC | QQYGSSPPFT | FGPGTKVDIKR |
| 2.7 | GASSRAT | GIPDRFSGSGSGTDFTLTISRLEPEDFAVYYC | QQYGSSPPFT | FGPGTKVDIKR |
| | AASSLQS | GVPSRFSGSGSGTDFTLTISSLQPEDFATYYC | QQSYSTPPT | FGQGTKVEIKR |
| 2.25 | AASSLQS | GVPSRFSGSGSGTDFTLTISSLQPEDFATYYC | QQSYNTPPT | FGQGTKVEIKR |
| 2.9 | AASSLQS | GVPSRFSGSGSGTDFTLTISSLQPEDFATYYC | QQSYSTPPT | FGQGTKVEIKR |
| | LGSNRAS | GVPDRFSGSGSGTDFTLKISRVEAEDVGVYYC | MQALQTIT | FGQGTRLEIKR |
| 2.19 | LGSNRAS | GVPDRFSGSGSGTDFTLKISRMEAEDVGVYYC | MQALQTIT | FGQGTRLEIKR |
| 1.14 | LGSYRAS | GVPDRFSGSGSGTDFTLKISRVEAEDAGVYFC | MQGLKTIT | FGQGTRLEIKR |
| | LIYAASS | LQSGVPSRFSGSGSGTDFTLTISSLQPEDFAT | YYCQQSYSTPPT | FGPGTKVDIKR |
| 2.12 | LIYAAST | LQSGVPSRFSGGGSGTDFTLTIRSLQPEDFAT | YYCQQSFNTPPT | FGPGTKVDIKR |

TABLE 7
Anti-PLA2 mAb Sequence Summary

| | Inhibit | Inhibit | | IC₅₀ | | | | | | |
|---|---|---|---|---|---|---|---|---|---|---|
| Ab | Mammalian | Bacterial | Isotype | (nM) | Bin | VH | DH | JH | VK | JK |
| 1.18 | 1 | 0 | hIgG2 | 0.78 | 1 | VH5-51 | D6-19 | JH4b | A30 | JK1 |
| 2.4 | 1 | 1 | hIgG4 | 0.91 | 1 | VH5-51 | D6-6 | JH3b | | |
| 2.25 | 1 | 1 | hIgG4 | 1.36 | 1 | VH5-51 | D6-6 | JH3b | O12 | JK1 |
| 2.9 | 1 | 0 | hIgG4 | 1.44 | 1 | VH5-51 | D6-6 | JH3b | O12 | JK1 |
| 1.5 | 1 | 1 | hIgG2 | 2.36 | 1 | VH5-51 | D2-8 | JH6b | | |
| 1.21 | 1 | 0 | hIgG2 | 3.64 | 1 | VH5-51 | | JH3b | A3 | JK1 |
| 1.14 | 1 | 1 | hIgG2 | 3.9 | 1 | VH5-51 | D1-26 | JH4b | A3 | JK5 |
| 2.16 | 1 | 0 | hIgG4 | 4.17 | 1 | VH5-51 | D1-26 | JH4b | | |
| 1.7 | 1 | 0 | hIgG2 | 4.73 | 1 | VH5-51 | D2-2 | JH6b | A27 | JK4 |
| 2.12 | 1 | 1 | hIgG4 | 4.82 | 1 | VH5-51 | D1-7 | JH3b | O12 | JK3 |
| 1.3 | 1 | 1 | hIgG2 | 6.07 | 1 | VH5-51 | | JH6b | | |
| 1.8 | 1 | 1 | hIgG2 | 6.56 | 1 | | | | | |
| 1.27 | 1 | 0 | hIgG2 | 9.4 | 1 | VH5-51 | D2-2 | JH6b | A27 | JK4 |
| 2.15 | 1 | 0 | hIgG4 | 14.33 | 2 | VH3-33 | D1-7 | JH3b | O12 | JK5 |
| 2.23 | 1 | 0 | hIgG4 | 14.4 | 2 | VH3-33 | D3-3 | JH4b | O12 | JK5 |
| 2.19 | 0 | 1 | hIgG4 | | 2 | VH3-33 | D1-1 | JH3b | A3 | JK5 |
| 2.7 | 0 | 1 | hIgG4 | | 1 | VH5-51 | D3-16 | JH3b | A27 | JK3 |
| 2.24 | 0 | 1 | hIgG4 | | | | | | | |

### Example 6

### Screening for Functional Inhibitors of PLA2 Enzyme

To identify antibodies that block PLA2 functional activity, the basic protocol for assaying enzyme activity in 96-well plates was modified for screening in 384-well plates. Fifty-eight (58) antibody candidates that had been found previously to block enzyme binding in ELISA format were screened. Several antibodies that block PLA2 functional activity were identified.

Basic Enzyme Assay

Briefly, the following three components were mixed in black 384-well plates: (1) 20 μl dIH₂O or KLH-supernatents or test media; (2) 20 μl enzyme diluted into dIH₂O; and (3) 20 μl Bis-BODIPY®-FLC₁₁-PC substrate diluted in 3× assay buffer. The plates were covered and incubated at room temperature for specified time(s). After incubation, 20 μl 40 mM EDTA was optionally added to stop reaction and the plates were read on the α-Fusion (Packard) using FITC filters, top read (excitation=485 nm; emission=530 nm).

Assay Development

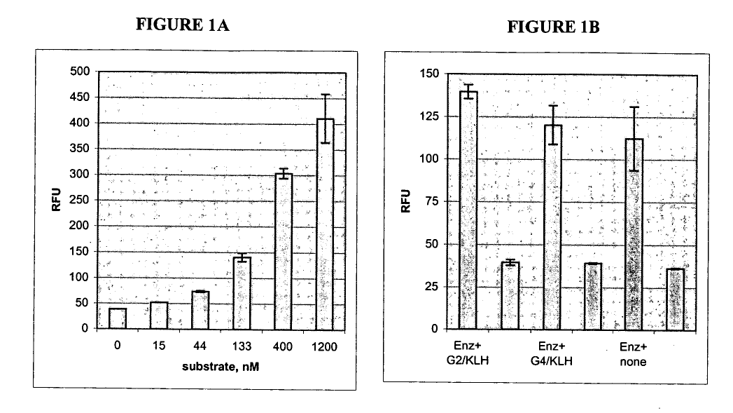

Initial experiments were performed to test the limit of detection of the substrate and the effect of KLH exhaust supernatant. Commercially available Porcine PLA2 was used as the positive control for testing the substrate, FIG. 1A shows a dose-response curve of titrating amounts of substrate incubated with 0.5 units enzyme. To test the effect of KLH supernatants, 0.5 units enzyme, 130 nM substrate, and 20 μl (or none) of KLH supernatant per well were incubated. Multiple time points were read and data from the 30 minute incubation is shown in FIG. 1B. KLH supernatent had minimal effects on the assay.

Bacterially-expressed PLA2. Bacterially-expressed enzyme is approximately four (4) times more active after Fxa-cleavage than before cleaving (data not shown.) The GST tag was cleaved from the bacterially-expressed GST-enzyme fusion protein using Fxa. Briefly, GST-enzyme fusion protein was incubated with 1 unit of Fxa in 1×Fxa buffer overnight at room temperature.

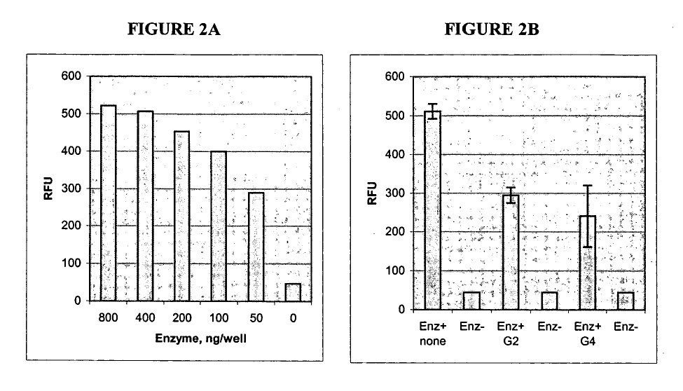

Titrating amounts of Fxa-cleaved bacterial-expressed enzyme were incubated with 400 nM Bis-BODIPY® substrate. Multiple time points were read and data from 10-minute incubation is shown in FIG. 2A. In order to test the effect of KLH supernatants, 0.4 μg Fxa-cleaved enzyme, 400 nM substrate and 20 μl (or none) KLH supernatant per well was incubated. Enzyme activity was slightly inhibited by 20 μl/well KLH exhaust supernatant from G2 and G4 (FIG. 2B). Ten-minute incubation is shown.

Competition Assay

The ability of the assay to detect inhibitors was tested using a competition assay. Briefly, 20 μl/well test hybridoma supernatant was added, followed by 20 μl enzyme diluted into dIH₂O. The plates were covered and incubated at 4° C. overnight. After incubation, 20 μl Bis-BODIPY® FLC₁₁ PC substrate was added. The plates were then covered and incubated at room temperature for 60 minutes. Following incubation, 20 μl 40 mM EDTA was added to stop the reaction and the plates were read on the α-Fusion using FITC filters.

Mouse sera from immunized mice was tested in duplicates. Sera (20 μl or none) was incubated with 0.2 μg Fxa-cleaved bacterially-expressed enzyme and 0.4 μM substrate per well. The plate was read 30 minutes after incubation. Both G2 and G4 sera were found to inhibit enzyme activity. Reading at other time points gave similar results.

Confirmation Screening

In a single 384-well plate, 58 hybridoma supernatants with both the mammalian PLA2 enzyme as well as with the Fxa-cleaved bacterial PLA2 enzyme were assayed. The mammalian enzyme was assayed in replicate and the bacterially-enzyme at single points. Control wells were also included for each enzyme.

Both enzymes, mammalian and bacterial expressions, gave good assay windows in the control wells. For the Fxa-cleaved bacterial cells, 150 ng enzyme was incubated with 400 nM substrate. For the mammalian enzyme, 0.5 μl CHO-supernatant was incubated with 400 nM substrate.

Hybridoma supernatant was tested for inhibition of enzyme activity. Hybridoma supernatant (20 μl/well) was incubated with 0.5 μl CHO-sup or 150 ng Fxa-cleave bacterial enzyme overnight. Substrate, at 400 nM final, was added, the plate was incubated and read at 30 minutes and at 60 minutes. EDTA was added after 60 minutes and read again. Data is shown for 60 minutes after EDTA addition. Table 8 summarizes the hits for inhibition of mammalian and bacterially-expressed enzymes. Hits that blocked enzyme activity gave 20 to 74% inhibition. Several antibodies that blocked mammalian and bacterial PLA2 functional activity were identified.

TABLE 8
Hits for inhibition of enzyme activity

| Clone | Mammalian | Bacterial |
|---|---|---|
| 1.3 | Inhibited | Inhibited |
| 1.5 | Inhibited | Inhibited |
| 1.7 | Inhibited | — |
| 1.8 | Inhibited | Inhibited |
| 1.14 | Inhibited | Inhibited |
| 1.18 | Inhibited | — |
| 1.21 | Inhibited | — |
| 1.27 | Inhibited | — |
| 2.4 | Inhibited | Inhibited |
| 2.7 | — | Inhibited |
| 2.9 | Inhibited | — |
| 2.12 | Inhibited | Inhibited |
| 2.15 | Inhibited | — |
| 2.16 | Inhibited | — |
| 2.19 | — | Inhibited |
| 2.23 | Inhibited | — |
| 2.24 | — | Inhibited |
| 2.25 | Inhibited | Inhibited |

### Example 7

### Measurement of PLA2 Activity by a Fluorescence Assay

A fluorescence assay was optimized for measuring Ca⁺²-dependent secretory PLA2. Bis-BODIPY® FL C₁₁-PC (Molecular Probes, Euguen, Oreg.) was used as substrate. The proximity of the BODIPY® FL fluorophores on adjacent phospholipid acyl chains results in self-quenching of fluorescence, which is alleviated by phospholipase A₁- or A₂-mediated release of a BODIPY® FL-labeled fatty acid. PLA2 activity was tested by measuring increased fluorescence using a fluorescein filter (485 nm excitation and 535 nm emission) on a fluorometer.

Briefly, 75 μl/well of 0.532 μM substrate in 1.33× assay buffer was added to a 96-well plate. 5 μl/well of the test compounds in 20% DMSO was added, followed by 20 μl of 5×PLA2 enzyme in Tris-Cl buffer, pH 7.6. Following incubation, the reaction mixture was stopped after 5 minutes at room temperature by adding 10 μl of EDTA to a final concentration of 10 mM. Fluorescence was measured on a microplate fluorometer using a fluorescein filter (485 nm excitation and 535 nm emission). Measurements were made within 15 to 30 minutes for reactions stopped by EDTA.

### Example 8

### Anti-PLA2 mAb Functional Assay

Purified antibodies, previously identified to bind and block PLA2 enzyme activity, were further characterized using a functional PLA2 assay described earlier. Four antibodies (mAbs 1.18, 2.4, 2.25, and 2.9) were found to inhibit PLA2 function at IC₅₀<2 nM. All of the antibodies block enzymes expressed in mammalian and bacterial cells.

Antibodies that were found to block PLA2 enzyme were then expressed in mammalian CHO cells (CHO-PLA2-MTX).

Affinity Rank order based on IC₅₀. Two separate experiments, a 6-point dose curve and a 12-point dose curve, were performed to rank the affinities of the antibodies. Data from the two dose curves concur. Data from the 12-point dose curve is shown below in Table 9.

TABLE 9
Rank order based on IC₅₀

| | |
|---|---|
| Rank order | Rank order |
| within isotype | across all isotypes |

| Ab | Isotype | IC₅₀ (nM) | Ab | Isotype | IC₅₀ (nM) |
|---|---|---|---|---|---|
| 1.18 | hIgG2 | 0.78 | 1.18 | hIgG2 | 0.78 |
| 1.5 | hIgG2 | 2.36 | 2.4 | hIgG4 | 0.91 |
| 1.21 | hIgG2 | 3.64 | 2.25 | hIgG4 | 1.36 |
| 1.14 | hIgG2 | 3.90 | 2.9 | hIgG4 | 1.44 |
| 1.7 | hIgG2 | 4.73 | 1.5 | hIgG2 | 2.36 |
| 1.3 | hIgG2 | 6.07 | 1.21 | hIgG2 | 3.64 |
| 1.8 | hIgG2 | 6.56 | 1.14 | hIgG2 | 3.90 |
| 1.27 | hIgG2 | 9.40 | 2.16 | hIgG4 | 4.17 |
| 2.4 | hIgG4 | 0.91 | 1.7 | hIgG2 | 4.73 |
| 2.25 | hIgG4 | 1.36 | 2.12 | hIgG4 | 4.82 |
| 2.9 | hIgG4 | 1.44 | 1.3 | hIgG2 | 6.07 |
| 2.16 | hIgG4 | 4.17 | 1.8 | hIgG2 | 6.56 |
| 2.12 | hIgG4 | 4.82 | 1.27 | hIgG2 | 9.40 |
| 2.15 | hIgG4 | 14.33 | 2.15 | hIgG4 | 14.33 |
| 2.23 | hIgG4 | 14.40 | 2.23 | hIgG4 | 14.40 |

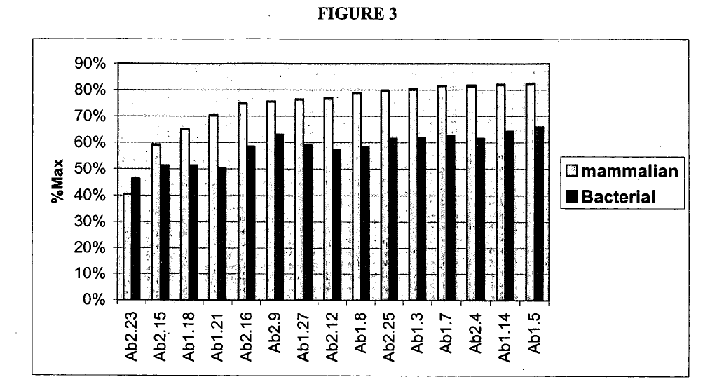

Six-point dose response curves for each antibody were tested against both mammalian and bacterial enzymes. The graph in FIG. 3 shows percent inhibition at the highest dose tested for each antibody. Replicas of four are plotted. SEM are as graphed.

### Example 9

### Biacore® Binding of Anti-PLA2 mAbs

To identify which antibodies bind the antigen PLA2 with the highest affinity, analysis was performed at 25° C. using a Biacore® 2000 biosensor (Biacore, Piscataway, N.J.) equipped with a research-grade CM5 sensor chip. In the immobilization, HBS-P was used as the running buffer; in the binding studies, HBS-P, with 12 mg/mL of each BSA and dextran, was used as sample and running buffers.

Briefly, antibody supernatants were diluted 1/10 and antibodies were captured individually on the IgG surfaces. After a one-minute wash step, buffer and PLA2 (300 nM) were injected serially (one-minute association, five minute dissociation) over each surface. Capture levels were measured from −20 to 0 seconds and the surfaces were regenerated with a 6-second pulse of 100 mM H₃PO₄.

From the initial screen, the supernatants could be divided into three classes: (1) high antibody titer, high antigen recognition (>50 RU Ab captured, >10 RU Ag response); (2) high antibody titer, low antigen recognition (>50 RU Ab captured, <10 RU Ag response); (3) low antibody titer, low or variable antigen recognition (<50 RU Ab captured, <10 RU Ag response). The capture levels and binding responses obtained for each antibody are shown in Table 10 below.

TABLE 10
Antibody capture levels and PLA2 binding responses

| Supernatant | Capture Level | Response |
|---|---|---|
| 1.1 | 963 | 181 |
| 1.2 | 968 | 64 |
| 1.3 | 639 | 53 |
| 1.4 | 948 | 36 |
| 1.5 | 484 | 70 |
| 1.6 | 761 | 141 |
| 1.7 | 918 | 118 |
| 1.8 | 1020 | 134 |
| 1.9 | 752 | 75 |
| 1.10 | 1020 | 237 |
| 1.11 | 124 | 12 |
| 1.12 | 466 | 24 |
| 1.13 | 788 | 64 |
| 1.14 | 573 | 74 |
| 1.15 | 607 | 2 |
| 1.16 | 729 | 38 |
| 1.17 | 534 | 72 |
| 1.18 | 90 | 6 |
| 1.19 | 625 | 42 |
| 1.20 | 248 | 3 |
| 1.21 | 415 | 61 |
| 1.22 | 342 | 47 |
| 1.23 | 977 | 2 |
| 1.24 | 446 | 20 |
| 1.25 | 3 | 1 |
| 1.26 | 809 | 41 |
| 1.27 | 700 | 78 |
| 1.28 | 146 | 7 |
| 2.1 | 458 | 25 |
| 2.2 | 418 | 0 |
| 2.3 | 432 | 7 |
| 2.4 | 334 | 40 |
| 2.5 | 195 | 28 |
| 2.6 | 547 | 13 |
| 2.7 | 612 | 126 |
| 2.8 | 108 | 0 |
| 2.9 | 304 | 20 |
| 2.10 | 506 | 74 |
| 2.11 | 14 | 0 |
| 2.12 | 720 | 98 |
| 2.13 | 331 | 47 |
| 2.14 | 201 | 0 |
| 2.15 | 997 | 37 |
| 2.16 | 212 | 26 |
| 2.17 | 366 | 0 |
| 2.18 | 622 | 43 |
| 2.19 | 708 | 52 |
| 2.20 | 311 | 72 |
| 2.21 | 815 | 40 |
| 2.22 | 61 | 6 |
| 2.23 | 392 | 7 |
| 2.24 | 141 | 1 |
| 2.25 | 226 | 18 |
| 2.26 | 423 | 51 |
| 2.27 | 364 | 22 |
| 2.28 | 522 | 21 |
| 2.29 | 89 | 0 |

Antibody Class II (shown in bold) and Class III (shown in italics) supernatants were re-screened using a 1/10 dilution for Class II and a ½ dilution for Class III. Capture time was increased and 750 nM PLA2 was injected to detect binding. The capture levels and binding responses obtained under these conditions are shown in Table 11. Only supernatants exhibiting PLA2 binding responses of >10 RU were considered viable candidates for high resolution analysis.

TABLE 11
Antibody capture levels and 750 nM PLA2 binding responses
obtained at ½ (Class III) and 1/10 (Class II) dilutions

| Supernatant | Capture Level | Response |
|---|---|---|
| 1.15 | 1460 | 0 |
| 1.18 | 165 | 14 |
| 1.20 | 630 | 0 |
| 1.23 | 1550 | 0 |
| 1.25 | 281 | 0 |
| 1.28 | 452 | 16 |
| 2.2 | 941 | 2 |
| 2.3 | 1050 | 18 |
| 2.8 | 449 | 9 |
| 2.11 | 150 | 0 |
| 2.14 | 567 | 4 |
| 2.17 | 851 | 21 |
| 2.22 | 123 | 16 |
| 2.23 | 939 | 35 |
| 2.24 | 379 | 0 |
| 2.29 | 278 | 5 |

Data were normalized based on the individual antibody capture levels and fit globally to a 1:1 interaction model. Antibodies 1.1, 1.10, 1.16, 2.18, 2.19 and 2.20 were found to be high affinity antibodies. Determined kinetic constants are summarized in Table 12.

Affinities were calculated from the ratio of the dissociation and association rates. The underlined dissociation rates were not floated in the data fitting process. Supernatants selected for the medium-resolution are shown in bold in Table 12 below.

TABLE 12
Affinity-ranked Antibodies from antibody supernatants

| Supernatant | ka (M − 1s− 1) | kd (s − 1) | KD(nM) | Rmax (RU) |
|---|---|---|---|---|
| 2.18 | 1.68E+04 | 1.00E−05 | 0.60 | 0.27 |
| 1.10 | 8.45E+04 | 5.69E−05 | 0.67 | 0.30 |
| 1.16 | 1.84E+04 | 1.65E−05 | 0.90 | 0.25 |
| 1.15 | 4.51E+03 | 1.00E−05 | 2.2 | 0.25 |
| 1.1 | 7.98E+04 | 2.53E−04 | 3.2 | 0.25 |
| 2.19 | 1.86E+04 | 1.15E−04 | 6.2 | 0.26 |
| 2.20 | 1.24E+05 | 9.96E−04 | 8.0 | 0.27 |
| 1.6 | 7.65E+04 | 6.92E−04 | 9.0 | 0.25 |
| 1.7 | 4.74E+04 | 4.51E−04 | 9.5 | 0.23 |
| 2.26 | 6.14E+04 | 5.99E−04 | 9.8 | 0.20 |
| 2.4 | 3.90E+04 | 4.03E−04 | 10 | 0.25 |
| 2.7 | 9.96E+04 | 1.04E−03 | 10 | 0.24 |
| 1.21 | 5.71E+04 | 6.30E−04 | 11 | 0.24 |
| 2.12 | 4.50E+04 | 6.56E−04 | 15 | 0.25 |
| 1.5 | 4.87E+04 | 7.13E−04 | 15 | 0.25 |
| 2.10 | 4.89E+04 | 7.85E−04 | 16 | 0.25 |
| 1.14 | 4.39E+04 | 7.91E−04 | 18 | 0.24 |
| 1.12 | 1.31E+04 | 2.38E−04 | 18 | 0.29 |
| 2.13 | 4.74E+04 | 8.64E−04 | 18 | 0.25 |
| 1.24 | 1.46E+04 | 2.93E−04 | 20 | 0.25 |
| 1.9 | 2.81E+04 | 5.71E−04 | 20 | 0.26 |
| 2.16 | 4.09E+04 | 8.75E−04 | 21 | 0.24 |
| 1.22 | 7.31E+04 | 1.67E−03 | 23 | 0.19 |
| 2.25 | 2.53E+04 | 5.76E−04 | 23 | 0.25 |
| 1.28 | 1.73E+04 | 4.31E−04 | 25 | 0.25 |
| 1.17 | 6.18E+04 | 1.55E−03 | 25 | 0.22 |
| 2.5 | 5.97E+04 | 1.56E−03 | 26 | 0.25 |
| 1.27 | 3.45E+04 | 9.22E−04 | 27 | 0.25 |
| 1.8 | 2.97E+04 | 8.32E−04 | 28 | 0.33 |
| 1.11 | 3.13E+04 | 9.28E−04 | 30 | 0.29 |
| 2.22 | 5.02E+04 | 1.67E−03 | 33 | 0.27 |
| 1.13 | 2.73E+04 | 9.85E−04 | 36 | 0.21 |
| 1.4 | 9.90E+03 | 3.97E−04 | 40 | 0.25 |
| 1.3 | 2.44E+04 | 9.92E−04 | 41 | 0.25 |
| 1.18 | 3.86E+04 | 1.92E−03 | 50 | 0.27 |
| 2.1 | 1.50E+04 | 8.11E−04 | 54 | 0.25 |
| 2.9 | 2.02E+04 | 1.10E−03 | 55 | 0.25 |
| 1.19 | 2.14E+04 | 1.29E−03 | 60 | 0.22 |
| 1.26 | 1.30E+04 | 8.46E−04 | 65 | 0.25 |
| 2.27 | 1.84E+04 | 1.31E−03 | 71 | 0.24 |
| 2.21 | 1.16E+04 | 8.99E−04 | 78 | 0.28 |
| 2.8 | 1.43E+04 | 1.35E−03 | 94 | 0.25 |
| 2.28 | 6.50E+03 | 6.43E−04 | 99 | 0.42 |
| 2.15 | 8.88E+03 | 9.02E−04 | 100 | 0.21 |
| 2.6 | 5846 | 6.39E−04 | 110 | 0.26 |
| 1.2 | 1.76E+04 | 2.03E−03 | 120 | 0.25 |
| 2.23 | 8.27E+03 | 9.69E−04 | 120 | 0.26 |
| 2.29 | 7.28E+03 | 1.50E−03 | 210 | 0.47 |
| 1.20 | 5.75E+03 | 1.27E−03 | 220 | 0.25 |
| 2.24 | 7.94E+03 | 1.84E−03 | 230 | 0.26 |
| 2.17 | 4.07E+03 | 9.79E−04 | 240 | 0.27 |
| 2.3 | 5.19E+03 | 1.48E−03 | 290 | 0.25 |
| 2.2 | 212E+03 | 1.19E−03 | 560 | 0.25 |
| 1.23 | 1.11E+03 | 9.67E−04 | 770 | 0.25 |
| 2.14 | 3.20E+03 | 4.18E−03 | 1300 | 0.25 |
| 1.25 | n.a. | n.a. | n.a. | n.a. |
| 2.11 | n.a. | n.a. | n.a. | n.a. |

### Example 10

### Mulitplexed Competitive Antibody Binning (MCAB) Assay

The mAbs were sorted into different bins based on cross-competition for antigen binding using the Mulitplexed Competitive Antibody Binning (MCAB) assay. The MCAB assay is based on the competitive binding of two Abs to one epitope on a single antigen molecule. U.S. application Ser. No. 10/309,419, filed Dec. 2, 2002, entitled “Antibody Characterization Based on Binding Characteristics,” Publication No. US-2003-0157730-A1. Prior to applying the MCAB method, antibodies in supernatants from the primary hybridoma cultures were identified and confirmed for antigen reactivity by ELISA or other methodology. Each antigen-immunoreactive Ab was used to form an antibody-antigen complex, in which the antibody is termed a “reference” Ab. In addition, each antibody was used as a “probe” Ab to determine bins based on competition. The Luminex® technology allows a probe antibody to be competed against all the reference antibody-antigen complexes simultaneously and provides the assay its multiplexing power.

Coupling of mouse anti-hIgG monoclonal antibodies to Luminex® beads. Briefly, an aliquot of each hybridoma supernatant (as reference Ab) from a panel of antigen-reactive hybridomas was incubated with a spectrally unique coded bead coupled to a mouse anti-hIgG mAb as described in the Luminex® 100 User's Manual, Version 1.7. Following activation, the beads were coupled to a mouse anti-hIgG mAb (Pharmingen, San Diego, Calif.) and incubated for 2 hours at room temperature or overnight at 4° C. After incubation, the coated beads were blocked and then counted using a Coulter cell counter.

Epitope binning. The bead-coupled reference Abs was then mixed and aliquotted into wells of a 96-well plate. Antigen was added to each well in order to form antibody-antigen complexes. Each antibody in supernatant (now as probe Ab) was added to individual wells. Finally, biotinylated mouse anti-hIgG mAb was added, followed by streptavidin-PE to detect binding of probe Abs.

Briefly, each set of bead-mouse anti-hIgG complexes were separately incubated with a reference Ab on a rotator at 4° C. overnight. After the reference Ab was captured, the bead-tagged mouse anti-hIgG-reference Ab complexes were pooled together, immediately added to each well of a 96-well filter plate, and aspirated. Next, 50 ng antigen was added to each well and incubated for 1 hour at room temperature. After a wash, 100 to 500 ng/ml of a probe Ab was added to each well and incubated for 2 hours at room temperature. Bound probe Ab was detected with 1 g/ml biotinylated version of the same monoclonal mouse anti-hIgG used for capturing the reference Ab. Finally, 0.5 μg/ml streptavidin-PE was added and incubated for 30 minutes at room temperature.

A parallel assay set without antigen was also performed as a background control for each mAb combination. The collection of beads in each well was then scanned with a Luminex® 100 to quantify the extent of binding of any given probe Ab to each of the multiplex antigen-reference-Ab-bead complexes. Positive signal expressed as relative fluorescence units indicated that the probe Ab was able to bind antigen bound to the reference Ab and therefore that the Ab pair do not compete for binding. Signal equivalent to background indicated that the probe Ab fails to bind antigen bound to the reference Ab and that the Ab pair compete for binding, and are therefore in the same bin. Antibodies in the same bin have the same or overlapping epitopes.

Seventeen (17) potently neutralizing antibodies were identified from a panel of antibodies against PLA2 in an assay in vitro. Performance of the MCAB assay identified two bins of neutralizing antibodies. (See Table 13). Bin 1 contained 14 antibodies and bin 2 contained 3 antibodies. 17 individual antibodies neutralizing the activity of PLA2 yielded only 2 germline VH genes. The most frequent gene, VH5-51 germline gene was expressed in 14 of the 17 antibodies analyzed, and was limited to bin 1. Selection of functional antibodies and binning showed antibodies in specific bins expressing the same Ig V_{H}, and in some cases, the same V_{H}DJ_{H} rearrangements.

TABLE 13
Anti-PLA2 mAbs Sequence/Binning Summary

| Clone | VH | DH | JH | VK | JK | Bin |
|---|---|---|---|---|---|---|
| 2.19.1 | VH3-33 | D1-1 | JH3B | A3 | JK5 | 2B |
| 2.15.1 | VH3-33 | D1-7 | JH3B | O12 | JK5 | 2A |
| 2.23.1 | VH3-33 | D3-3 | JH4B | O12 | JK5 | 2C |
| 2.7.1 | VH5-51 | D3-16 | JH3B | A27 | JK3 | 1 |
| 2.9.1 | VH5-51 | D6-6 | JH3B | O12 | JK1 | 1 |
| 2.25.1 | VH5-51 | D6-6 | JH3B | O12 | JK1 | 1 |
| 2.4.1 | VH5-51 | D6-6 | JH3B | | | 1 |
| 1.18.1 | VH5-51 | D6-19 | JH4B | A30 | JK1 | 1 |
| 1.7.1 | VH5-51 | D2-2 | JH6B | A27 | JK4 | 1 |
| 1.27.1 | VH5-51 | D2-2 | JH6B | A27 | JK4 | 1 |
| 1.5.1 | VH5-51 | D2-8 | JH6B | | | 1 |
| 2.12.1 | VH5-51 | D1-7 | JH3B | O12 | JK3 | 1 |
| 2.23.1 | VH5-51 | D3-3 | JH4B | O12 | JK5 | 1 |
| 1.3.1 | VH5-51 | | JH6B | | | 1 |
| 1.21.1 | VH5-51 | | JH3B | A3 | JK1 | 1 |
| 1.14.1 | VH5-51 | D1-26 | JH4B | A3 | JK5 | 1 |
| 2.16.1 | VH5-51 | D1-26 | JH4B | | | 1 |

Fine Resolution Epitope Mapping. Fine resolution epitope mapping of two unique antibodies from each of the two bins was performed to confirm the binning results. Peptide scans of the overlapping peptides were used for epitope mapping using SPOTs technology (Sigma Genosys, Inc.). Briefly, the entire 157-amino acid sequence of PLA2 was custom-synthesized as a series of overlapping 12-mer oligopeptides, offset by two residues, thereby generating a library of arrayed, overlapping peptides on a nylon membrane (Sigma Genosys, Inc.). Antibodies were tested for binding to the arrayed oligopeptides using standard conditions for Western blotting. Binding of mAbs to the membrane-bound peptides was assessed by ELISA using HRP-conjugated secondary antibody followed by enhanced chemiluminescence (ECL). Spots showing binding corresponded to oligopeptides containing the epitope. Anti-PLA2 mAbs 2.12, 2.25, 2.15, and 2.23 were shown to recognize linear epitopes by dot blot.

Anti-PLA2 mAbs 2.15.1 and 2.23.1 mapped to bin 2 and both bound to the same linear epitope, GPAENK (amino acids 119-124 of SEQ ID NO: 2). Anti-PLA2 mAbs 2.12.1 and 2.25.1 both mapped to bin 1. While anti-PLA2 mAb 2.12.1 mapped to a larger epitope, PQFLCEPD (amino acids 153-160 of SEQ ID NO: 2), anti-PLA2 mAb 2.25.1 mapped to the minimal epitope of PQFL (amino acids 153-156 of SEQ ID NO: 2), which is contained within the epitope of anti-PLA2 mAb 2.21.1, thereby confirming the underlying molecular basis of the results of the MCAB assay. Anti-PLA2 mAbs in bin 1 and bin 2 had conserved V_{H} gene usage. For example, bin 1 antibodies all use V_{H}5-51 but have different CDR3 sequences and light chain composition. Bin 2 antibodies all use V_{H}3-33 and have different CDR3 sequences and light chain composition. Correlations have been observed between bins based on binding competition, antibody activity in functional assays, and composition of the variable region for other, but not all, antigen targets (data not shown).

It appears that only a few members of the germ line repertoire are used to form the corresponding paratope, and for each antigenic epitope a limited number of L- and H-chain genes can pair to form a specific paratope. Bin1 mAbs were found to share the same heavy and light chain gene usage. Although there were differences in CDR3 and the length of FR2 in the light chain, both bound overlapping epitopes. Bin 2 mAbs share the same heavy and light chain gene usage. Although there are differences in CDR3 and the length of FR2 in the light chain, both bound to identical epitopes.

### Example 11

### Panning of Phase Display Random Peptide Library with Anti-PLA2 mAb 12.2.

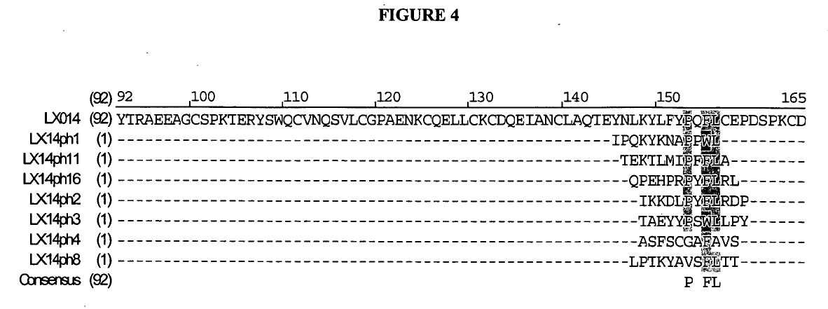

Ph.D.-12™ phage display library (New England BioLabs) of random peptide 12-mers fused to minor coat protein (pIII) of M13 phage was panned against anti-PLA2 mAb 2.12. Specific binders were selected by ELISA assay and sequenced. Peptide sequences for the specific binders were then aligned to the PLA2 antigen sequence. The alignment of the peptide sequences of specific binders of anti-PLA2 mAb 2.12 is shown in FIG. 4. It was determined that the consensus sequence PXFL aligned to residues 153-156 of the PLA2 sequence set out in SEQ ID NO: 2.

### Example 12

### In Vitro Transcription/Translation

Four monoclonal antibodies were tested for binding to in vitro transcription (IVT) products linked to Luminex® beads. All constructs were expressed as 6Xhis tag fusion proteins. Binding to the full-length IVT product was observed, however, no binding was observed to fragments 1-36PLA2His and 1-63PLA2His, indicating that the epitope is beyond amino acid 63 and at the C-terminal region of the molecule.

TABLE 14

Heavy chain Alignment of Bin 1 Antibodies

| | | | |
|---|---|---|---|
| SEQ ID NO. | Ab | FR1 | CDR1 |

| | | | |
|---|---|---|---|
| 217 | VH5-51 | EVQLVQSGAEVKKPGESLKISCKGS | GYSFTSYWIG |
| 218 | 2.25 | EVQLVQSGAEVKKPGESLKISCKGS | GYSFISYWIA |
| 219 | 2.12 | EVQLVQSGAEVKKPGESLKISCKGS | GYNFITYWIA |

| | | |
|---|---|---|
| Ab | FR2 | CDR2 |

| | | |
|---|---|---|
| VH5-51 | WVRQMPGKGLEWMG | IIYPGDSDTRYSPSFQG |
| 2.25 | WVRQMPGKGLEWMG | IIYPGDSDARYSPSFQG |
| 2.12 | WVRQMPGKGLEWMG | IIYPGDSDTRYSPSFQG |

| | | |
|---|---|---|
| Ab | FR3 | CDR3 |

| | | |
|---|---|---|
| VH5-51 | QVTISADKSISTAYLQWSSLKASDTAMYYCAR | WGQGTMVTVSSA |
| 2.25 | QVTISADKSISTAYLQWSSLKASDTAMYYCAR | TTQDTMVTVSSA |
| 2.12 | QVTISADKSISTAYLQWSSLKASDTAMYYCAL | WGQRTMETVSSA |

TABLE 15

Kappa chain Alignment of Bin 1 Antibodies

| | | | |
|---|---|---|---|
| SEQ ID NO. | Ab | FR1 | CDR1 |

| | | | |
|---|---|---|---|
| 220 | O-12 | DIQMTQSPSSLSASVGDRVTITC | RASQSISSYLN |
| 221 | 2.25 | DIQMTQSPSSLSASVGDRVTITC | RASQSISSYLN |
| 222 | 2.12* | DIQMTQSPSSLSASVGDRVTITC | RASQSIGSYLN |

| | | |
|---|---|---|
| Ab | FR2 | CDR2 |

| | | |
|---|---|---|
| 0-12 | WYQQKPGKA###PKLLIY | AASSLQS |
| 2.25 | WYQQKPGKA###PKLLIY | AASSLQS |
| 2.12* | WYQKPGKPGKGPKLLIY | AASSLQST |

| | | | |
|---|---|---|---|
| Ab | FR3 | CDR3 | J |

| | | | |
|---|---|---|---|
| O-12 | GVPSRFSGSGSGTDFTLTISSLQPEDFATYYC | QQSYSTPPT | FGQGTKVEIKR |
| 2.25 | GVPSRFSGSGSGTDFTLTISSLQPEDFATYYC | QQSYNTPPT | FGQGTKVEIKR |
| | | | (JK1) |
| 2.12* | GVPSRFSGSGSGTDFTLTISSLRPEDFATYYC | QQSFNTPPT | FGPGTKVDIKR |
| | | | (JK3) |

*2.12 shows sequence duplication and insertion of 3 residues in FR2

### Example 13

### Uses of Anti-PLA2 Antibodies and Antibody Conjugates for Treatment of Inflammation

Antibodies specific to PLA2 antigens such as anti-PLA2 antibodies are useful in targeting cardiovascular cells expressing such antigens, for example, as lipid lowering agents in the treatment of atherosclerosis and restenosis.

Treatment of mice with anti-PLA2 antibodies. To determine in vivo effects of anti-PLA2 antibody in the treatment of cardiovascular injuries, vascular injury model knock-out mice are injected over a predetermined period of time with an effective amount of anti-PLA2 antibody. The vascular injury is induced in mice by placing a cuff around the carotid artery thereby causing an inflammatory infiltrate of PLA2, and thus, inflammation and a thickening of the walls of the carotid artery similar to that found in patients with atherosclerosis and restenosis is created. At periodic times during the treatment with the anti-PLA2 antibodies, the mice are monitored to determine the state of the vascular injury. A significant reduction of the vascular injury is noted.

### Example 14

### Treatment of Humans with Anti-PLA2 Antibodies

To determine the in vivo effects of anti-PLA2 antibody treatment in human patients with inflammatory diseases such as atherosclerosis and restenosis, such human patients are injected over a predetermined period of time with an effective amount of a fully human anti-PLA2 antibody. At periodic times during the treatment, the human patients are monitored to determine whether significant reduction of inflammation is noted.

A patient having atherosclerosis treated with anti-PLA2 antibodies is found to have a lower level of lipids than untreated patients and/or patients treated with control antibodies. Control antibodies that are used include antibodies of the same isotype as the anti-PLA2 antibodies tested and further, may not have the ability to bind to PLA2.

### Example 15

### Treatment with Anti-PLA2 Antibody Conjugates

To determine the in vivo effects of anti-PLA2 antibody conjugates, human patients or animals exhibiting inflammatory diseases, such as atherosclerosis or restenosis, are injected over a predetermined period of time with an effective amount of anti-PLA2 antibody conjugate. In one embodiment, the anti-PLA2 antibody conjugate administered is maytansine-anti-PLA2 antibody conjugate or radioisotope-anti-PLA2 antibody conjugate. At periodic times during the treatment, the human patients or animals are monitored to determine whether their inflammation is reduced, in particular, whether a significant reduction of the vascular injury is noted.

A human patient or animal exhibiting atherosclerosis or restenosis and undergoing treatment with either maytansine-anti-PLA2 antibody or radioisotope-anti-PLA2 antibody conjugates is found to have a lower level of vascular injury and inflammation when compared to a control patient or animal with atherosclerosis or restenosis and undergoing treatment with control antibody conjugates, such as control maytansine-antibody or control radioisotope-antibody. Control maytansine-antibodies that may be used include conjugates comprising maytansine linked to antibodies of the same isotype of the anti-PLA2 antibodies, but more specifically, not having the ability to bind to PLA2 antigen. Control radioisotope-antibodies that may be used include conjugates comprising radioisotope linked to antibodies of the same isotype of the anti-PLA2 antibodies, but more specifically, not having the ability to bind to PLA2 antigen.

### Example 16

### Use of Anti-PLA2 Antibodies as a Diagnostic Agent

Detection of PLA2 Antigen in a Sample.

An Enzyme-Linked Immunosorbent Assay (ELISA) for the detection of PLA2 antigen in a sample are developed. In the assay, wells of a microtiter plate, such as a 96-well microtiter plate or a 384-well microtiter plate, are adsorbed for several hours with a first fully human monoclonal antibody directed against the antigen. The immobilized antibody serves as a capture antibody for any of the antigen that may be present in a test sample. The wells are rinsed and treated with a blocking agent such as milk protein or albumin to prevent nonspecific adsorption of the analyte.

Subsequently the wells are treated with a test sample suspected of containing the antigen, or with a solution containing a standard amount of the antigen. Such a sample may be, for example, a serum sample from a subject suspected of having levels of circulating antigen considered to be diagnostic of a pathology.

After rinsing away the test sample or standard, the wells are treated with a second fully human monoclonal anti-PLA2 antibody that is labeled by conjugation with biotin. The labeled anti-PLA2 antibody serves as a detecting antibody. After rinsing away excess second antibody, the wells are treated with avidin-conjugated horseradish peroxidase (HRP) and a suitable chromogenic substrate. The concentration of the antigen in the test samples is determined by comparison with a standard curve developed from the standard samples.

This ELISA assay provides a highly specific and very sensitive assay for the detection of the PLA2 antigen in a test sample.

Determination of PLA2 Antigen Concentration in Patients.

A sandwich ELISA is developed to quantify PLA2 levels in human serum. The two fully human monoclonal anti-PLA2 antibodies used in the sandwich ELISA, recognize different epitopes on the PLA2 molecule (data not shown). The ELISA is performed as follows: 50 μl of capture anti-PLA2 antibody in coating buffer (0.1 M NaHCO₃, pH 9.6) at a concentration of 2 μg/ml is coated on ELISA plates (Fisher). After incubation at 4° C. overnight, the plates are treated with 200 μl of blocking buffer (0.5% BSA, 0.1% Tween 20, 0.01% Thimerosal in PBS) for 1 hour at 25° C. The plates are washed (3×) using 0.05% Tween 20 in PBS (washing buffer, WB). Normal or patient sera (Clinomics, Bioreclaimation) are diluted in blocking buffer containing 50% human serum. The plates are incubated with serum samples overnight at 4° C., washed with WB, and then incubated with 100 μl/well of biotinylated detection anti-PLA2 antibody for 1 hour at 25° C. After washing, the plates are incubated with HRP-Streptavidin for 15 minutes, washed as before, and then treated with 100 μl/well of o-phenylenediamine in H₂O₂ (Sigma developing solution) for color generation. The reaction is stopped with 50 μl/well of H₂SO₄ (2M) and analyzed using an ELISA plate reader at 492 nm. Concentration of PLA2 antigen in serum samples is calculated by comparison to dilutions of purified PLA2 antigen using a four-parameter curve-fitting program.

Staging of Inflammatory Diseases in a Patient.

It will be appreciated that based on the results set forth and discussed in the Examples above, through use of the embodiments of the invention, it is possible to stage a cardiovascular injury in a subject based on expression levels of the PLA2 antigen. For a given type of injury, samples of blood are taken from subjects diagnosed as being at various stages in the progression of the disease, and/or at various points in the therapeutic treatment of the disease. The concentration of the PLA2 antigen present in the blood samples is determined using a method that specifically determines the amount of the antigen that is present. Such a method includes an ELISA method, such as the method described in the Examples above. Using a population of samples that provides statistically significant results for each stage of progression or therapy, a range of concentrations of the antigen that may be considered characteristic of each stage is designated.

In order to stage the progression of the disease in a subject under study, or to characterize the response of the subject to a course of therapy, a sample of blood is taken from the subject and the concentration of the PLA2 antigen present in the sample is determined. The concentration so obtained is used to identify in which range of concentrations the value falls. The range so identified correlates with a stage of progression or a stage of therapy identified in the various populations of diagnosed subjects, thereby providing a stage in the subject under study.

The foregoing written specification is considered to be sufficient to enable one skilled in the art to practice the invention. The embodiments of the invention described herein are not to be limited in scope by the construct deposited, since the deposited embodiment is intended as a single illustration of certain aspects of the invention and any constructs that are functionally equivalent are within the scope of this invention. The deposit of material herein does not constitute an admission that the written description herein contained is inadequate to enable the practice of any aspect of the invention, including the best mode thereof, nor is it to be construed as limiting the scope of the claims to the specific illustrations that it represents.

### Incorporation by Reference

All references cited herein, including patents, patent applications, papers, text books, and the like, and the references cited therein, to the extent that they are not already, are hereby incorporated herein by reference in their entirety. In addition, the following references are also incorporated by reference herein in their entirety, including the references cited in such references:

### Equivalents

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A human monoclonal antibody that binds to phospholipase A2 (PLA2) and comprises a heavy chain having an amino acid sequence selected from the group consisting of SEQ ID NOS: 3, 5, 7, 9, 11, 13, 15, 17, 19, 21, 23, 25, 27, 29, 30, and 31.

2. The antibody of claim 1, further comprising a light chain having an amino acid sequence selected from the group consisting of SEQ ID NOS: 4, 6, 8, 10, 12, 14, 16, 18, 20, 22, 24, 26, and 28.

3. The human monoclonal antibody of claim 2, wherein the heavy chain has an amino sequence comprising the sequence of SEQ ID NO:3 and the light chain has an amino sequence comprising the sequence of SEQ ID NO:4.

4. The human monoclonal antibody of claim 2, wherein the heavy chain has an amino sequence comprising the sequence of SEQ ID NO:5 and the light chain has an amino sequence comprising the sequence of SEQ ID NO:6.

5. The human monoclonal antibody of claim 2, wherein the heavy chain has an amino sequence comprising the sequence of SEQ ID NO:7 and the light chain has an amino sequence comprising the sequence of SEQ ID NO:8.

6. The human monoclonal antibody of claim 2, wherein the heavy chain has an amino sequence comprising the sequence of SEQ ID NO:9 and the light chain has an amino sequence comprising the sequence of SEQ ID NO:10.

7. The human monoclonal antibody of claim 2, wherein the heavy chain has an amino sequence comprising the sequence of SEQ ID NO:11 and the light chain has an amino sequence comprising the sequence of SEQ ID NO:12.

8. The human monoclonal antibody of claim 2, wherein the heavy chain has an amino sequence comprising the sequence of SEQ ID NO:13 and the light chain has an amino sequence comprising the sequence of SEQ ID NO:14.

9. The human monoclonal antibody of claim 2, wherein the heavy chain has an amino sequence comprising the sequence of SEQ ID NO:15 and the light chain has an amino sequence comprising the sequence of SEQ ID NO:16.

10. The human monoclonal antibody of claim 2, wherein the heavy chain has an amino sequence comprising the sequence of SEQ ID NO:17 and the light chain has an amino sequence comprising the sequence of SEQ ID NO:18.

11. The human monoclonal antibody of claim 2, wherein the heavy chain has an amino sequence comprising the sequence of SEQ ID NO:19 and the light chain has an amino sequence comprising the sequence of SEQ ID NO:20.

12. The human monoclonal antibody of claim 2, wherein the heavy chain has an amino sequence comprising the sequence of SEQ ID NO:21 and the light chain has an amino sequence comprising the sequence of SEQ ID NO:22.

13. The human monoclonal antibody of claim 2, wherein the heavy chain has an amino sequence comprising the sequence of SEQ ID NO:23 and the light chain has an amino sequence comprising the sequence of SEQ ID NO:24.

14. The human monoclonal antibody of claim 2, wherein the heavy chain has an amino sequence comprising the sequence of SEQ ID NO:25 and the light chain has an amino sequence comprising the sequence of SEQ ID NO:26.

15. The human monoclonal antibody of claim 2, wherein the heavy chain has an amino sequence comprising the sequence of SEQ ID NO:27 and the light chain has an amino sequence comprising the sequence of SEQ ID NO:28.

16. An antibody immobilized on an insoluble matrix, wherein the antibody is the antibody of claim 2.

17. A method for assaying the level of phospholipase A2 (PLA2) in a patient sample, wherein said method comprises the use of the anti-PLA2 antibody of claim 2 for detection of the level of PLA2 in the assay of a patient sample.

18. The method according to claim 17 wherein the patient sample is blood.

19. A composition comprising the antibody of claim 2, or a binding fragment thereof, and a pharmaceutically acceptable carrier.

20. A method of effectively treating inflammatory conditions, comprising:
selecting an animal in need of treatment for an inflammatory condition; and
administering to said animal a therapeutically effective dose of an antibody, or binding fragment thereof, that specifically binds to phospholipase A2 (PLA2).

21. The method of claim 20, wherein said animal is human.

22. The method of claim 20, where said antibody is a fully human monoclonal antibody.

23. The method of claim 20, wherein said inflammatory condition is selected from the group consisting of: inflammatory and degenerative disorders stemming from inflammatory reactions in the joints, skin, and blood vessels, arthritis, psoriasis, asthma, Alzheimer's disease, atherosclerosis, and restenosis.

24. The method of claim 20, wherein the antibody is the antibody of claim 2.

25. A method of effectively treating restinosis, comprising:
selecting an animal in need of treatment for an inflammatory condition; and
administering to said animal a therapeutically effective dose of an antibody, or binding fragment thereof, that specifically binds to phospholipase A2 (PLA2).

26. The method of claim 25, wherein said animal is human.

27. The method of claim 25, wherein said antibody is a fully human monoclonal antibody.

28. The method of claim 25, wherein the antibody is the antibody of claim 2.
